(19) United States
(12) Patent Application Publication
(10) Pub. No.: US 2024/0317854 A1
(43) Pub. Date: Sep. 26, 2024

(54) METHODS FOR TREATING CANCER WITH ANTI-ILT3 ANTIBODIES

(71) Applicants: Philip E. Brandish, Needham, MA (US); Mei Chen, Audubon, PA (US); Andrey Loboda, Canton, MA (US); Michael Nebozhyn, III, Colmar, PA (US); Cai Wu, Philadelphia, PA (US); Daping Zhang, Kirkland, WA (US); Jie Zhang-Hoover, Lexington, MA (US)

(72) Inventors: Philip E. Brandish, Needham, MA (US); Mei Chen, Audubon, PA (US); Andrey Loboda, Canton, MA (US); Michael Nebozhyn, III, Colmar, PA (US); Cai Wu, Philadelphia, PA (US); Daping Zhang, Kirkland, WA (US); Jie Zhang-Hoover, Lexington, MA (US)

(73) Assignee: Merck Sharp & Dohme LLC, Rahway, NJ (US)

(21) Appl. No.: 18/550,537

(22) PCT Filed: Mar. 17, 2022
(86) PCT No.: PCT/US2022/020714

§ 371 Date: Sep. 14, 2023

Related U.S. Application Data
(60) Provisional application No. 63/163,779, filed on Mar. 19, 2021.

(51) Int. Cl. C07K 16/28; A61K 31/337; A61K 31/7068; A61K 39/00; A61K 39/395; A61P 35/00

(52) U.S. Cl. CPC C07K 16/2803; A61K 31/337; A61K 31/7068; A61K 39/3955; A61P 35/00; C07K 16/2818; A61K 2039/505; A61K 2039/507; A61K 2039/545

(57) ABSTRACT

This disclosure relates to methods for treating solid tumors in a patient identified as having metastatic triple negative breast cancer, glioblastoma, metastatic pancreatic ductal adenocarcinoma, metastatic soft tissue sarcoma, or metastatic non-squamous non-small cell lung carcinoma, comprising administering an anti-ILT3 antigen binding protein, or antigen binding fragment, and an anti-PD1 antigen binding protein, or antigen binding fragment, to the patient every three weeks (Q3W).

### SEQUENCE LISTING

The instant application contains a Sequence Listing which has been submitted electronically in ASCII format and is hereby incorporated by reference in its entirety. Said ASCII copy, created on Mar. 11, 2022, is named 25212-WO-PCT_SL.txt and is 410,136 bytes in size.

### FIELD

This disclosure relates to methods for treating cancer in a subject comprising administering an anti-ILT3 antigen binding protein, including an antibody or antigen binding fragment, alone or in combination to the subject.

### BACKGROUND

Immune checkpoint therapies targeting the PD-1 axis have resulted in groundbreaking improvements in clinical responses in multiple human cancers (Brahmer et al., N Engl J Med 2012, 366: 2455-65; Garon et al. N Engl J Med 2015, 372: 2018-28; Hamid et al., N Engl J Med 2013, 369: 134-44; Robert et al., Lancet 2014, 384: 1109-17; Robert et al., N Engl J Med 2015, 372: 2521-32; Robert et al., N Engl J Med 2015, 372: 320-30; Topalian et al., N Engl J Med 2012, 366: 2443-54; Topalian et al., J Clin Oncol 2014, 32: 1020-30; Wolchok et al., N Engl J Med 2013, 369: 122-33). Immune therapies targeting the PD-1 axis include monoclonal antibodies directed to the PD-1 receptor (KEYTRUDA (pembrolizumab), Merck and Co., Inc., Kenilworth, NJ, USA and OPDIVO (nivolumab), Bristol-Myers Squibb Company, Princeton, NJ, USA) and those that bind to the PD-L1 ligand (MPDL3280A; TECENTRIQ (atezolizumab), Genentech, San Francisco, CA, USA; IMFINZI (durvalumab), AstraZeneca Pharmaceuticals LP, Wilmington, DE; BAVENCIO (avelumab), Merck KGaA, Darmstadt, Germany). Both therapeutic approaches have demonstrated anti-tumor effects in numerous cancer types.

However, certain cancer indications are refractory to treatment with PD-1 or PD-L1 inhibitors. A role for myeloid cells in the molecular epidemiology of resistance to checkpoint inhibitors, including pembrolizumab, has been reported, and ILT3 is strongly associated with that myeloid signature. Studies have documented the infiltration of tumors with myeloid cells and an association of that feature with immunosuppression and resistance to checkpoint inhibitors (Kumar et al. The Nature of Myeloid-Derived Suppressor Cells in the Tumor Microenvironment. Trends Immunol. 2016 March; 37(3):208-220; Solito et al. Myeloid-derived suppressor cell heterogeneity in human cancers. Ann N Y Acad Sci. 2014; 1319:47-65; Messmer et al. Tumor-induced myeloid dysfunction and its implications for cancer immunotherapy. Cancer Immunol Immunother. 2015; 64:1-13). In patients with previously treated metastatic bladder cancer, a high baseline circulating monocytic Myeloid-Derived Suppressor Cell (MDSC) count was associated with a shorter overall survival after treatment with nivolumab compared to patients with a low MDSC count (Sharma, P., et al. Nivolumab in metastatic urothelial carcinoma after platinum therapy (CheckMate 275): a multicentre, single-arm, phase 2 trial. Lancet Oncol. 2017 March; 18(3):312-322). Furthermore, De Goeje et al. have observed an inverse correlation between the level of ILT3 expression on circulating MDSCs and patient survival in NSCLC (de Goeje, P. L., et al.). Thus, there exists a need for additional therapies in the treatment of cancers that are resistant to treatment with immune checkpoint inhibitors.

Immunoglobulin-like transcript 3 (ILT3), designated CD85k and also known as Leukocyte Immunoglobulin-Like Receptor subfamily B member 4 (LILRB4) and Leukocyte Immunoglobulin-like Receptor 5 (LIR-5), is a type I membrane protein that contains cytoplasmic immunoreceptor tyrosine-based inhibition motif (ITIM) motifs and is involved in the down-regulation of immune responses (Cella et al., J Exp Med. 185 (10): 1743-51 (1997); Samaridis et al., Eur J Immunol. 27 (3): 660-665 (1997). Expression of ILT3 is up-regulated on tolerogenic dendritic cells. This gene is a member of the leukocyte immunoglobulin-like receptor (LIR) family, which is found in a gene cluster at chromosomal region 19q13.4. The encoded protein belongs to the subfamily B class of LIR receptors, which contain two or four extracellular immunoglobulin domains, a transmembrane domain, and two to four ITIMs.

ILT3 is expressed by myeloid-derived suppressor cells (MDSCs) and correlates with survival in patients with non-small cell lung cancer. Oncoimmunology. 2015; 4(7):e1014242). Murine studies of an anti-ILT3 antibody in NOD scid gamma humanized mouse model systems reveal its ability to reduce tumor burden and shift cellular phenotypes to a more activated state (see WO2019/099597).

The ILT3 pathway may be a key regulatory element responsible for the induction and maintenance of tumor immune tolerance. Inhibitors of ILT3 may provide an innovative and tractable method to treat malignancies alone or in combination with inhibitors of the PD-1/PD-L1 axis.

### SUMMARY

Embodiment 1: the disclosure provides a pharmaceutical composition comprising from 0.02 mg to 2250 mg of an anti-ILT3 antigen binding protein or antigen binding fragment and a pharmaceutically acceptable excipient.
Embodiment 2: the disclosure provides a method of treating cancer in a subject in need thereof comprising administering to a subject a therapeutically effective dose of a pharmaceutical composition comprising from 0.02 mg to 2250 mg of an anti-ILT3 antigen binding protein or antigen binding fragment and a pharmaceutically acceptable excipient.
Embodiment 3: the method of embodiment 2, comprising further administering to the subject a therapeutically effective dose of an anti-PD1 antigen binding protein, or antigen binding fragment in combination, serially, or simultaneously with the pharmaceutical composition.
Embodiment 4: the method of any one of embodiments 2 and 3, wherein the cancer is metastatic triple negative breast cancer (mTNBC).
Embodiment 5: a method of embodiment 4, wherein before the administration step, the subject is identified as: a) having a PD-L1 enriched tumor, wherein the PD-L1 enriched tumor is a tumor identified as having a CPS score of ≥1.
Embodiment 5.1: a method of embodiment 4, wherein before the administration step, the subject is identified as:
a) having a PD-L1 enriched tumor, wherein the PD-L1 enriched tumor is a tumor identified as having a CPS score of ≥1; and
b) having received no prior systemic therapy for mTNBC.
Embodiment 6: The method of any one of embodiments 2-3, wherein the cancer is recurrent non-operable glioblastoma multiforme (GBM).
Embodiment 7: The method of embodiment 6, wherein, before the administration step, the subject is identified as:
a) having a histologically confirmed diagnosis of GBM;
b) having received a standard first-line treatment for GBM including surgery and radiation therapy with or without chemotherapy and evidence of disease recurrence or pression by magnetic resonance imaging (MRI);
c) having time elapsed from prior treatment;
d) having Karnofsky performance status (KPS)≥80 within 7 days before start of study treatment;
e) being neurologically stable; and
f) having a known status of 06-methylguanine-DNA methyltransferase (MGMT) methylation and isocitrate dehydrogenase (IDH).
Embodiment 7.1: The method of embodiment 6, wherein, before the administration step, the subject is identified as:
a) having a histologically confirmed diagnosis of GBM; and
b) having received a standard first-line treatment for GBM including surgery and radiation therapy with or without chemotherapy and evidence of disease recurrence or pression by magnetic resonance imaging (MRI).
Embodiment 8: The method of any one of embodiments 2-3, wherein the cancer is metastatic pancreatic ductal adenocarcinoma (mPDAC).
Embodiment 9: The method of embodiment 8, wherein before the administration step, the subject is identified as:
a) having a histologically confirmed diagnosis of mPDAC and has received no prior systemic therapy for mPDAC; and
b) having an albumin level of ≥3.0 g/dL in a serum sample.
Embodiment 9.1: The method of embodiment 8, wherein before the administration step, the subject is identified as:
a) having a histologically confirmed diagnosis of mPDAC and has received no prior systemic therapy for mPDAC; and
b) having received no prior systemic therapy for mPDAC.
Embodiment 10: The method of any one of embodiments 2-3, wherein the cancer is metastatic soft tissue sarcoma (mSTS).
Embodiment 11: The method of embodiment 10, wherein before the administration step, the subject is identified as having progressed after receiving one prior line of systemic treatment for advanced mSTS.
Embodiment 11.1: The method of embodiment 10, wherein before the administration step, the subject is identified as:
a) having a histologically confirmed diagnosis of locally advanced or metastatic mSTS; and
b) having progressed after receiving one prior line of systemic treatment for advanced mSTS.
Embodiment 12. The method of any one of embodiments 2-3, wherein the cancer is metastatic non-squamous non-small cell lung carcinoma (mNSCLC).
Embodiment 13: The method of embodiment 12, wherein before the administration step, the subject is identified as:
a) having a histologically confirmed diagnosis of Stage IV or recurrent non-operable non-squamous non-small cell lung carcinoma (NSCLC);
b) not having epidermal growth factor receptor (EGFR), anaplastic lymphoma kinase (ALK), or c-ros oncogene 1 (ROS1) directed therapy indicated as a primary therapy; and
c) not having received prior systemic treatment for metastatic NSCLC.
Embodiment 13.1: The method of embodiment 12, wherein before the administration step, the subject is identified as:
a) having a histologically confirmed diagnosis of Stage IV or recurrent non-operable non-squamous non-small cell lung carcinoma (NSCLC);
b) not eligible for an approved targeted therapy;
c) having progressed on treatment with an anti-PD-(L)1 monoclonal antibody (mAb) administered either as monotherapy, or in combination with other checkpoint inhibitors or other therapies; and
d) having progressive disease (PD) during/after platinum doublet chemotherapy.
Embodiment 13.2: The method of embodiment 12, wherein before the administration step, the subject is identified as:
a) having a histologically confirmed diagnosis of Stage IV or recurrent non-operable non-squamous non-small cell lung carcinoma (NSCLC);
b) not having epidermal growth factor receptor (EGFR), anaplastic lymphoma kinase (ALK), or c-ros oncogene 1 (ROS1) directed therapy indicated as a primary therapy;
c) not having received prior systemic treatment for metastatic NSCLC; and
d) having a PD-L1 enriched tumor, wherein the PD-L1 enriched tumor is a tumor identified as having a CPS score of ≥1.
Embodiment 14: The method of any one of embodiments 2-13, wherein the subject is a human.
Embodiment 15: The method of any one of embodiments 2-14 or the pharmaceutical composition of embodiment 1, wherein the anti-ILT3 antigen-binding protein or antigen-binding fragment is an anti-ILT3 antibody or antigen-binding fragment.
Embodiment 16: The method or pharmaceutical composition of embodiment 15, wherein the antibody or antigen binding fragment that binds human immunoglobulin-like transcript 3 (ILT3) comprising:
a heavy chain (HC) having a variable heavy domain (VH) comprising a complementarity determining region (HC-CDR) 3 having an amino acid sequence selected from the group consisting of SEQ ID NO: 20, 47, 55, 63, 71, 79, 87, 95, and 103, or having an amino acid sequence that has 3, 2, or 1 differences with an amino acid sequence selected from the group consisting of SEQ ID NO: 20, 47, 55, 63, 71, 79, 87, 95, and 103.
Embodiment 17: The method or pharmaceutical composition of embodiment 15, wherein the anti-ILT3 antibody or antigen binding fragment comprises:
(a) a heavy chain (HC) having a variable heavy domain (VH) comprising a complementarity determining region (HC-CDR) 1 having the amino acid sequence set forth in SEQ ID NO: 15, 45, 53, 61, 69, 77, 85, 93, or 101; an HC-CDR2 having the amino acid sequence set forth in SEQ ID NO: 16, 46, 54, 62, 69, 78, 86, 94, or 102; and an HC-CDR3 having the amino acid sequence set forth in SEQ ID NO: 21, 47, 55, 63, 71, 79, 87, 95, or 103; and, variants thereof wherein one or more of the HC-CDRs has one, two, or three amino acid substitutions, additions, deletions, or combinations thereof; and
(b) a light chain (LC) having a variable light domain (VL) comprising a complementarity determining region (LC-CDR) 1 having the amino acid sequence set forth in SEQ ID NO: 25, 48, 56, 64, 72, 80, 88, 96, or 104; an LC-CDR2 having the amino acid sequence set forth in SEQ ID NO: 41, 49, 57, 65, 73, 81, 89, 97, or 105; and an LC-CDR3 having the amino acid sequence set forth in SEQ ID NO: 42, 50, 58, 66, 74, 82, 90, 98, or 106; and, variants thereof wherein one or more of the LC-CDRs has one, two, or three amino acid substitutions, additions, deletions, or combinations thereof.
Embodiment 18: The method or pharmaceutical composition of embodiment 17, wherein
(a) the HC-CDR1 has the amino acid sequence set forth in SEQ ID NO: 15; the HC-CDR2 has the amino acid sequence set forth in SEQ ID NO: 17, 18, or 19; the HC-CDR3 has the amino acid sequence set forth in SEQ ID NO: 21; and
(b) the LC-CDR1 has the amino acid sequence set forth in SEQ ID NO: 32, 33, 34, 35, 36, 37, 38, 39, or 40; the LC-CDR2 has the amino acid sequence set forth in SEQ ID NO: 41; and, the LC-CDR3 has the amino acid sequence set forth in SEQ ID NO: 42.
Embodiment 19: The method or pharmaceutical composition of embodiment 18, wherein
(a) the HC-CDR1 has the amino acid sequence set forth in SEQ ID NO: 15; the HC-CDR2 has the amino acid sequence set forth in SEQ ID NO: 18; and the HC-CDR3 has the amino acid sequence set forth in SEQ ID NO: 21; and
(b) the LC-CDR1 has the amino acid sequence set forth in SEQ ID NO: 39; the LC-CDR2 has the amino acid sequence set forth in SEQ ID NO: 41; and, the LC-CDR3 has the amino acid sequence set forth in SEQ ID NO: 42.
Embodiment 20: The method or pharmaceutical composition of any one of embodiments 17-19, wherein the VH comprises a framework selected from the group consisting of human V_{H}1, V_{H}2, V_{H}3, V_{H}4, VHS, and V_{H}6, and variants thereof having 1, 2, 3, 4, 5, 6, 7, 8, 9, or 10 amino acid substitutions, additions, deletions, or combinations thereof, and, the VL comprises a framework selected from the group consisting of human V_{κ}1, V_{κ}2, V_{κ}3, V_{κ}4, V_{κ}5, V_{κ}6, V_{λ}1, V_{λ}2, V_{λ}3, V_{λ}4, V_{λ}5, V_{λ}6, V_{λ}7, V_{λ}8, V_{λ}9, and V_{λ}10, and variants thereof having 1, 2, 3, 4, 5, 6, 7, 8, 9, or 10 amino acid substitutions, additions, deletions, or combinations thereof.
Embodiment 21: The method or pharmaceutical composition of any one of embodiments 17-20, wherein the antibody comprises an HC having a human IgG1, IgG2, IgG3, or IgG4 HC constant domain or variant thereof having 1, 2, 3, 4, 5, 6, 7, 8, 9, or 10 amino acid substitutions, additions, deletions, or combinations thereof compared to the amino acid sequence of the native IgG1, IgG2, IgG3, or IgG4 isotype constant domain.
Embodiment 22: The method or pharmaceutical composition of embodiment 20 or 21, wherein the antibody comprises an LC having a human kappa or lambda LC constant domain or variant thereof comprising 1, 2, 3, 4, 5, 6, 7, 8, 9, or 10 amino acid substitutions, additions, deletions, or combinations thereof compared to the amino acid sequence of the native human kappa or lambda light chain constant domain.
Embodiment 23: The method or pharmaceutical composition of embodiment 19, wherein the antibody comprises:
(i) a V_{H} having a framework selected from human V_{H}1, V_{H}2, V_{H}3, V_{H}4, V_{H}5, and V_{H}6 and a human IgG1 or IgG4 HC constant domain or variant thereof comprising 1, 2, 3, 4, 5, 6, 7, 8, 9, or 10 amino acid substitutions, additions, deletions, or combinations thereof compared to the amino acid sequence of the native IgG1 or IgG4 isotype HC constant domain; and,
(ii) a V_{L} having a framework selected from human V_{κ}1, V_{κ}2, V_{κ}3, V_{κ}4, V_{κ}5, V_{κ}6, V_{λ}1, V_{λ}2, V_{λ}3, V_{λ}4, V_{λ}5, V_{λ}6, V_{λ}7, V_{λ}8, V_{λ}9, and V_{λ}10 and a human kappa or lambda LC constant domain or variant thereof comprising 1, 2, 3, 4, 5, 6, 7, 8, 9, or 10 amino acid substitutions, additions, deletions, or combinations thereof compared to the amino acid sequence of the native human kappa or lambda LC constant domain.
Embodiment 24: The method or pharmaceutical composition of embodiment 20, wherein the antibody or antigen binding fragment comprises a V_{H} and a VL having the amino acid sequences set forth in SEQ ID NO: 13 and SEQ ID NO: 14, respectively; SEQ ID NO: 43 and SEQ ID NO: 44, respectively; SEQ ID NO: 51 and SEQ ID NO: 52, respectively; SEQ ID NO: 59 and SEQ ID NO: 60, respectively; SEQ ID NO: 67 and SEQ ID NO: 68, respectively; SEQ ID NO: 75 and SEQ ID NO: 76, respectively; SEQ ID NO: 83 and SEQ ID NO: 84, respectively; SEQ ID NO: 91 and SEQ ID NO: 92, respectively; or SEQ ID NO: 99 and SEQ ID NO: 100, respectively.
Embodiment 25: The method or pharmaceutical composition of embodiment 20, wherein the antibody or antigen binding fragment comprises a V_{H} having the amino acid sequence set forth in SEQ ID NO: 115, 116, 117, 121, 122, or 123 and a V_{L} having the amino acid sequence set forth in SEQ ID NO: 124, 125, 126, 127, 128, 129, 130, 131, 132, 133, 134, 135, 136, 137, 138, or 139.
Embodiment 26: The method or pharmaceutical composition of embodiment 25, wherein the antibody or antigen binding fragment comprises a V_{H} having the amino acid sequence set forth in SEQ ID NO: 116 and a V_{L} having the amino acid sequence set forth in SEQ ID NO: 138.
Embodiment 27: The method or pharmaceutical composition of any one of embodiments 23-26, wherein the antibody comprises a heavy chain (HC) constant domain comprising the amino acid sequence set forth in SEQ ID NO: 7, 8, 9, 10, or 11.
Embodiment 28: The method or pharmaceutical composition of any one of embodiments 23-26, wherein the antibody comprises a light chain (LC) constant domain comprising the amino acid sequence set forth in SEQ ID NO: 12.
Embodiment 29: The method or pharmaceutical composition of any one of embodiments 23-26, wherein the antibody comprises a heavy chain (HC) comprising the amino acid sequence of SEQ ID NO: 140, 141, 142, 146, 147, 148, 165, 166, 167, 168, 172, 173, 174, 175, 176, 180, 181, 182, 183, 184, 185, 189, 190, or 191.
Embodiment 30: The method or pharmaceutical composition of any one of embodiments 23-29, wherein the antibody comprises a light chain (LC) comprising the amino acid sequence set forth in SEQ ID NO: 149, 150, 151, 152, 153, 154, 155, 156, 157, 158, 159, 160, 161, 162, 163, or 164.
Embodiment 31: The method or pharmaceutical composition of embodiment 23, wherein the antibody comprises a heavy chain (HC) comprising the amino acid sequence set forth in SEQ ID NO: 141 and a light chain (LC) comprising the amino acid sequence set forth in SEQ ID NO: 163, and variants thereof wherein the HC lacks a C-terminal Lysine residue or a C-terminal glycine-lysine.
Embodiment 32: The method of any one of embodiments 2-31, wherein the anti-PD1 antigen binding protein or antigen binding fragment is an anti-PD-1 antibody or antigen binding fragment.
Embodiment 33: The method of embodiment 32, wherein the anti-PD-1 antibody or antigen-binding fragment comprises:
(a) light chain complementarity determining regions (CDRs) comprising a sequence of amino acids as set forth in SEQ ID NOs: 224, 225 and 226 and heavy chain CDRs comprising a sequence of amino acids as set forth in SEQ ID NOs: 227, 228, and 229; or
(b) light chain CDRs comprising a sequence of amino acids as set forth in SEQ ID NOs: 230, 231 and 232 and heavy chain CDRs comprising a sequence of amino acids as set forth in SEQ ID NOs: 233, 234, and 235.
Embodiment 34: The method of any of embodiments 32-33, wherein the anti-PD-1 antibody or antigen-binding fragment comprises:
(a) a heavy chain variable region comprising a sequence of amino acids as set forth in SEQ ID NO: 236, or a variant of SEQ ID NO: 236, and
(b) a light chain variable region comprising:
(i) a sequence of amino acids as set forth in SEQ ID NO: 237, or a variant of SEQ ID NO: 237,
(ii) a sequence of amino acids as set forth in SEQ ID NO: 238, or a variant of SEQ ID NO: 238, or
(iii) a sequence of amino acids as set forth in SEQ ID NO: 239, or a variant of SEQ ID NO: 239.
Embodiment 35: The method of any one of embodiments 32-34, wherein the anti-PD-1 antibody or antigen-binding fragment comprises a heavy chain variable region comprising a sequence of amino acids as set forth in SEQ ID NO: 236 and a light chain variable region comprising a sequence of amino acids as set forth in SEQ ID NO: 237.
Embodiment 36: The method of any one of embodiments 32-35, wherein the anti-PD-1 antibody or antigen-binding fragment is a monoclonal antibody comprising:
(a) a heavy chain comprising a sequence of amino acids as set forth in SEQ ID NO: 28, or a variant of SEQ ID NO: 240, and
(b) a light chain comprising a sequence of amino acids as set forth in SEQ ID NO: 241, a variant of SEQ ID NO: 241, SEQ ID NO: 242, a variant of SEQ ID NO: 242, SEQ ID NO: 243, or a variant of SEQ ID NO: 243.
Embodiment 37: The method of any one of embodiments 32-36, wherein the anti-PD-1 antibody or antigen-binding fragment is a monoclonal antibody comprising a heavy chain comprising a sequence of amino acids as set forth in SEQ ID NO: 240 and a light chain comprising a sequence of amino acids as set forth in SEQ ID NO: 241.
Embodiment 38: The method of embodiment 37, wherein the anti-PD1 antibody or antigen binding fragment comprises heavy chain variable domain complementarity determining regions (HC-CDR) 1, 2, and 3, and light chain variable domain complementarity determining regions (LC-CDR) 1, 2, and 3, wherein:
the HC-CDR1 comprises the amino acid sequence set forth in SEQ ID NO: 249; the HC-CDR2 comprises the amino acid sequence set forth in SEQ ID NO: 250; the HC-CDR3 comprises the amino acid sequence set forth in SEQ ID NO: 251; and the LC-CDR1 comprises the amino acid sequence set forth in SEQ ID NO: 244; the LC-CDR2 comprises the amino acid sequence set forth in SEQ ID NO: 245; and the LC-CDR3 comprises the amino acid sequence set forth in SEQ ID NO: 246.
Embodiment 39: The method of embodiment 37, wherein the anti-PD1 antibody or antigen binding fragment has a heavy chain variable region comprising the amino acid sequence set forth in SEQ ID NO: 252 and a light chain variable region comprising the amino acid sequence set forth in SEQ ID NO: 247.
Embodiment 40: The method of embodiment 37, wherein the anti-PD-1 antibody or antigen-binding fragment is a monoclonal antibody comprising a heavy chain comprising a sequence of amino acids as set forth in SEQ ID NO: 253 and a light chain comprising a sequence of amino acids as set forth in SEQ ID NO: 248.
Embodiment 41: The method or pharmaceutical composition of any one of embodiments 2-40, wherein the therapeutically effective amount of the anti-ILT3 antigen binding protein is from about 7.5 mg to about 2250 mg and the therapeutically effective amount of the anti-PD1 antigen binding protein is about 200 mg.
Embodiment 42: The method or pharmaceutical composition of any one of embodiments 2-41, wherein the therapeutically effective amount of the anti-ILT3 antigen binding protein is about 750 mg and the therapeutically effective amount of the anti-PD1 antigen binding protein is about 200 mg.
Embodiment 43: The method of any one of embodiments 2-42, wherein the anti-PD-1 antibody or antigen binding fragment and the anti-ILT3 antibody or antigen binding fragment are administered every three weeks (Q3W) of a 21-day cycle.
Embodiment 44: The method of any one of embodiments 4-43, comprising administering a taxane.
Embodiment 45: The method of embodiment 44, wherein the taxane is paclitaxel.
Embodiment 46: The method of embodiment 45, comprising administering the paclitaxel on days 1, 8 and 15 of a 28 day cycle.
Embodiment 47: The method of any one of embodiments 45-46, wherein the amount of paclitaxel administered on each administration day is about 90 mg/m².
Embodiment 48: The method of any one of embodiments 6-43, comprising administering nab-paclitaxel and gemcitabine.
Embodiment 49: The method of embodiment 48, comprising administering nab-paclitaxel in an amount of about 125 mg/m² via IV infusion and gemcitabine in an amount of about 1000 mg/m² via IV infusion on Days 1, 8 and 15 of a 28 day cycle.
Embodiment 50: The method of any one of embodiments 8-43, comprising administering
a) pemetrexed in an amount of about 500 mg/m² via IV infusion every three weeks (Q3W);
b) carboplatin with desired dose of area under the cure (AUC), administered via IV infusion Q3W for 4 administrations (up to about 3 months); and
c) pemetrexed in amount of about 500 mg/m², administered via IV infusion Q3W for 4 administrations (up to about 3 months), followed by maintenance therapy with pemetrexed in an amount of about 500 mg/m² via IV infusion.
Embodiment 51: The method of any of embodiments 2-50, wherein the anti-ILT3 antibody or antigen-binding fragment is administered to the patient by intravenous administration.
Embodiment 52: The method of any of embodiments 2-51, wherein the anti-PD-1 antibody or antigen-binding fragment is administered to the patient by intravenous or subcutaneous administration.
Embodiment 53: The method or pharmaceutical composition of any one of embodiments 2-52, wherein the pharmaceutical composition comprises an amount of anti-ILT3 antigen binding protein or antigen binding fragment selected from the group consisting of: 7.5 mg; 25 mg; 75 mg; 225 mg; 750 mg; and 2250 mg.
Embodiment 54: The method or pharmaceutical composition of embodiment 53, wherein the amount of anti-ILT3 antigen binding protein or antigen binding fragment is 7.5 mg.
Embodiment 55: The method or pharmaceutical composition of embodiment 53, wherein the amount of anti-ILT3 antigen binding protein or antigen binding fragment is 25 mg.
Embodiment 56: The method or pharmaceutical composition of embodiment 53, wherein the amount of anti-ILT3 antigen binding protein or antigen binding fragment is 75 mg.
Embodiment 57: The method or pharmaceutical composition of embodiment 53, wherein the amount of anti-ILT3 antigen binding protein or antigen binding fragment is 225 mg.
Embodiment 58: The method or pharmaceutical composition of embodiment 53, wherein the amount of anti-ILT3 antigen binding protein or antigen binding fragment is 750 mg.
Embodiment 59: The method or pharmaceutical composition of embodiment 53, wherein the amount of anti-ILT3 antigen binding protein or antigen binding fragment is 2250 mg.
Embodiment 60: The method or pharmaceutical composition of any one of embodiments 2-59, wherein the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein:
(a) the HC-CDR1 comprises the amino acid sequence set forth in SEQ ID NO: 15; the HC-CDR2 comprises the amino acid sequence set forth in SEQ ID NO: 17; the HC-CDR3 comprises the amino acid sequence set forth in SEQ ID NO: 21; the LC-CDR1 comprises the amino acid sequence set forth in SEQ ID NO: 36; the LC-CDR2 comprises the amino acid sequence set forth in SEQ ID NO: 41; and the LC-CDR3 comprises the amino acid sequence set forth in SEQ ID NO: 42;
(b) the HC-CDR1 has the amino acid sequence set forth in SEQ ID NO: 15; the HC-CDR2 has the amino acid sequence set forth in SEQ ID NO: 18; the HC-CDR3 has the amino acid sequence set forth in SEQ ID NO: 21; the LC-CDR1 has the amino acid sequence set forth in SEQ ID NO: 37; the LC-CDR2 has the amino acid sequence set forth in SEQ ID NO: 41; and, the LC-CDR3 has the amino acid sequence set forth in SEQ ID NO: 42;
(c) the HC-CDR1 has the amino acid sequence set forth in SEQ ID NO: 15; the HC-CDR2 has the amino acid sequence set forth in SEQ ID NO: 19; the HC-CDR3 has the amino acid sequence set forth in SEQ ID NO: 21; the LC-CDR1 has the amino acid sequence set forth in SEQ ID NO: 38; the LC-CDR2 has the amino acid sequence set forth in SEQ ID NO: 41; and, the LC-CDR3 has the amino acid sequence set forth in SEQ ID NO: 42;
(d) the HC-CDR1 has the amino acid sequence set forth in SEQ ID NO: 15; the HC-CDR2 has the amino acid sequence set forth in SEQ ID NO: 18; the HC-CDR3 has the amino acid sequence set forth in SEQ ID NO: 21; the LC-CDR1 has the amino acid sequence set forth in SEQ ID NO: 39; the LC-CDR2 has the amino acid sequence set forth in SEQ ID NO: 41; and, the LC-CDR3 has the amino acid sequence set forth in SEQ ID NO: 42;
(e) the HC-CDR1 has the amino acid sequence set forth in SEQ ID NO: 15; the HC-CDR2 has the amino acid sequence set forth in SEQ ID NO: 17; the HC-CDR3 has the amino acid sequence set forth in SEQ ID NO: 21; the LC-CDR1 has the amino acid sequence set forth in SEQ ID NO: 40; the LC-CDR2 has the amino acid sequence set forth in SEQ ID NO: 41; and, the LC-CDR3 has the amino acid sequence set forth in SEQ ID NO: 42.
Embodiment 61: The method or pharmaceutical composition of embodiment 60, wherein the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 comprises the amino acid sequence set forth in SEQ ID NO: 15; the HC-CDR2 comprises the amino acid sequence set forth in SEQ ID NO: 17; the HC-CDR3 comprises the amino acid sequence set forth in SEQ ID NO: 21; the LC-CDR1 comprises the amino acid sequence set forth in SEQ ID NO: 36; the LC-CDR2 comprises the amino acid sequence set forth in SEQ ID NO: 41; and the LC-CDR3 comprises the amino acid sequence set forth in SEQ ID NO: 42.
Embodiment 62: The method or pharmaceutical composition of embodiment 60, wherein the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 has the amino acid sequence set forth in SEQ ID NO: 15; the HC-CDR2 has the amino acid sequence set forth in SEQ ID NO: 18; the HC-CDR3 has the amino acid sequence set forth in SEQ ID NO: 21; the LC-CDR1 has the amino acid sequence set forth in SEQ ID NO: 37; the LC-CDR2 has the amino acid sequence set forth in SEQ ID NO: 41; and, the LC-CDR3 has the amino acid sequence set forth in SEQ ID NO: 42.
Embodiment 63: The method or pharmaceutical composition of embodiment 60, wherein the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 has the amino acid sequence set forth in SEQ ID NO: 15; the HC-CDR2 has the amino acid sequence set forth in SEQ ID NO: 19; the HC-CDR3 has the amino acid sequence set forth in SEQ ID NO: 21; the LC-CDR1 has the amino acid sequence set forth in SEQ ID NO: 38; the LC-CDR2 has the amino acid sequence set forth in SEQ ID NO: 41; and, the LC-CDR3 has the amino acid sequence set forth in SEQ ID NO: 42.
Embodiment 64: The method or pharmaceutical composition of embodiment 60, wherein the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 has the amino acid sequence set forth in SEQ ID NO: 15; the HC-CDR2 has the amino acid sequence set forth in SEQ ID NO: 18; the HC-CDR3 has the amino acid sequence set forth in SEQ ID NO: 21; the LC-CDR1 has the amino acid sequence set forth in SEQ ID NO: 39; the LC-CDR2 has the amino acid sequence set forth in SEQ ID NO: 41; and, the LC-CDR3 has the amino acid sequence set forth in SEQ ID NO: 42.
Embodiment 65: The method or pharmaceutical composition of embodiment 60, wherein the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 has the amino acid sequence set forth in SEQ ID NO: 15; the HC-CDR2 has the amino acid sequence set forth in SEQ ID NO: 17; the HC-CDR3 has the amino acid sequence set forth in SEQ ID NO: 21; the LC-CDR1 has the amino acid sequence set forth in SEQ ID NO: 40; the LC-CDR2 has the amino acid sequence set forth in SEQ ID NO: 41; and, the LC-CDR3 has the amino acid sequence set forth in SEQ ID NO: 42.
Embodiment 66: The method or pharmaceutical composition of any one of embodiments 2-59, wherein the anti-ILT3 antigen binding protein or antigen binding fragment comprises:
(a) a heavy chain of SEQ ID NO: 140 and a light chain of SEQ ID NO: 149;
(b) a heavy chain of SEQ ID NO: 146 and a light chain of SEQ ID NO: 151;
(c) a heavy chain of SEQ ID NO: 141 and a light chain of SEQ ID NO: 150;
(d) a heavy chain of SEQ ID NO: 141 and a light chain of SEQ ID NO: 163;
(e) a heavy chain of SEQ ID NO: 144 and a light chain of SEQ ID NO: 150.
Embodiment 67: The method or pharmaceutical composition of embodiment 66, wherein the anti-ILT3 antigen binding protein or antigen binding fragment comprises a heavy chain of SEQ ID NO: 140 and a light chain of SEQ ID NO: 149.
Embodiment 68: The method or pharmaceutical composition of embodiment 66, wherein the anti-ILT3 antigen binding protein or antigen binding fragment comprises a heavy chain of SEQ ID NO: 146 and a light chain of SEQ ID NO: 151.
Embodiment 69: The method or pharmaceutical composition of embodiment 66, wherein the anti-ILT3 antigen binding protein or antigen binding fragment comprises a heavy chain of SEQ ID NO: 141 and a light chain of SEQ ID NO: 150.
Embodiment 70: The method or pharmaceutical composition of embodiment 66, wherein the anti-ILT3 antigen binding protein or antigen binding fragment comprises a heavy chain of SEQ ID NO: 141 and a light chain of SEQ ID NO: 163.
Embodiment 71: The method or pharmaceutical composition of embodiment 66, wherein the anti-ILT3 antigen binding protein or antigen binding fragment comprises a heavy chain of SEQ ID NO: 144 and a light chain of SEQ ID NO: 150.
Embodiment 72: A pharmaceutical composition comprising from 0.02 mg to 2250 mg of an anti-ILT3 antigen binding protein or antigen binding fragment and a pharmaceutically acceptable excipient for use in the methods of any one of embodiments 2-71.
Embodiment 73: Use of a pharmaceutical composition comprising from 0.02 mg to 2250 mg of an anti-ILT3 antigen binding protein or antigen binding fragment and a pharmaceutically acceptable excipient in the manufacture of a medicament for use in the methods of any one of embodiments 2-71.

The summary of the technology described above is non-limiting and other features and advantages of the technology will be apparent from the following detailed description, and from the claims.

### BRIEF DESCRIPTION OF THE DRAWINGS

FIG. 1A is a schematic diagram depicting a dose escalation and cohort expansion study design. Participants may receive up to 35 cycles of anti-ILT3 antibody in both monotherapy and combination arms. Higher dose levels will be tested until target saturation in fresh tumor biopsies is achieved unless MTD/MAD is reached before. See Table 1 for details. Participants may transition to Arm 2 if disease progression is seen in monotherapy and after consultation with and approval by the Sponsor. Participants who cross-over to combination treatment will be eligible to receive a maximum of 35 cycles of combination treatment irrespective of the number of cycles or dose of anti-ILT3 antibody received in monotherapy.

FIG. 1B is a schematic diagram depicting study design for anti-ILT3 antibody monotherapy patients crossing over to receiving combination therapy with an anti-PD-1 antibody. If participants in Arm 1 (anti-ILT3 mAb monotherapy) experience disease progression, they may be eligible for cross-over to combination treatment (Arm 2). Participants can only cross-over once they have completed the DLT period for Arm 1 and upon cross-over may receive the highest dose of anti-ILT3 mAb that has passed the DLT evaluation period in Arm 2 (combination) at the time of cross-over. Cross-over is optional, is at the discretion of the investigator, and requires the Sponsor's approval. Disease progression, toxicity or 35 administrations (24 months of treatment): participants who cross-over to combination treatment will be eligible to receive a maximum of 35 cycles of combination treatment irrespective of the number of cycles or dose of anti-ILT3 mAb received in monotherapy.

FIG. 2 is a schematic diagram depicting cohorts of particular solid tumor indications to be treated with ILT3 antibody and PD-1 antibody. An IA may be conducted after the first 15 participants (Cohorts B, C, and D) or 20 participants (Cohort A) have their second post-baseline imaging assessment. If 8 or fewer responses (Cohort A), 3 or fewer responses (Cohort C), or 1 or fewer responses (Cohorts B and D) are observed, enrollment in the cohort may be stopped early. An mTPI design will be applied to determine the safety and tolerability of the chemotherapy combinations.

### DETAILED DESCRIPTION OF THE DISCLOSURE

### Definitions and Abbreviations

As used throughout the specification and appended claims, the following abbreviations apply:
1L first line
2L second line
AE adverse event
ATD accelerated titration design
AUC area under the concentration-time curve
BICR blinded independent central review
C Cycle
CDR complementarity determining region
CI confidence interval
CPS combined positive score
D Day
Discon discontinuation
DL dose level
DLT dose-limiting toxicity
DOR duration of response
ECOG Eastern Cooperative Oncology Group
FFPE formalin-fixed paraffin-embedded
FR framework region
FU follow-up
GBM glioblastoma multiforme
IA interim analysis
IgG immunoglobulin G
IHC immunohistochemistry or immunohistochemical
IA interim analysis
IV intravenous
mAb monoclonal antibody
MAD maximum administered dose
MDSC myeloid-derived suppressor cells
MPS modified proportion score
MRI magnetic resonance imaging
MTD maximum tolerated dose
mTPI Modified Toxicity Probability Interval Design
NSCLC non-small cell lung cancer
NCI CTCAE National Cancer Institute—Common Terminology Criteria for Adverse Events
ORR objective response rate
OS overall survival
PD progressive disease
PDAC pancreatic ductal adenocarcinoma
PD-1 programmed death 1 (a.k.a. programmed cell death-1 and programmed death receptor 1)
PD-L1 programmed cell death 1 ligand 1
PD-L2 programmed cell death 1 ligand 2
Pembro pembrolizumab
PFS progression free survival
PK pharmacokinetic
Q2W one dose every two weeks
Q3W one dose every three weeks
Q6W one dose every six weeks
Q9W one dose every 9 weeks
Q12W one dose every 12 weeks
SAE serious adverse event
SC subcutaneous
STS soft tissue sarcoma
TNBC triple negative breast cancer
VH immunoglobulin heavy chain variable region
VL immunoglobulin light chain variable region

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

Reference to “or” indicates either or both possibilities unless the context clearly dictates one of the indicated possibilities. In some cases, “and/or” was employed to highlight either or both possibilities.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

The term “about”, when modifying the quantity (e.g., mg) of a substance or composition, or the value of a parameter characterizing a step in a method, or the like, refers to variation in the numerical quantity that can occur, for example, through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like. In certain embodiments, “about” means a variation of ±10%.

As used herein, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, an anti-PD-1 antigen binding fragment that consists essentially of a recited amino acid sequence may also include one or more amino acids, including substitutions of one or more amino acid residues, which do not materially affect the properties of the binding compound.

“Administration” and “treatment,” as they apply to an animal, human, experimental subject, cell, tissue, organ, or biological fluid, refer to contact of an exogenous pharmaceutical, therapeutic, diagnostic agent, or composition to the animal, human, subject, cell, tissue, organ, or biological fluid. “Treat” or “treating” cancer, as used herein, means to administer an anti-ILT3 antigen binding protein (e.g., an antibody) or antigen-binding fragment, alone or in combination with an anti-PD-1 antigen binding protein or antigen binding fragment to a subject having cancer, including but not limited to a solid tumor (e.g., metastatic triple negative breast cancer (mTNBC), recurrent non-operable glioblastoma (GBM), metastatic pancreatic ductal adenocarcinoma (mPDAC), metastatic soft tissue sarcoma (mSTS), metastatic non-squamous non-small cell lung carcinoma (mNSCLC)), or diagnosed with a solid tumor disease (e.g., mTNBC, GBM, mPDAC, mSTS, or mNSCLC) to achieve at least one positive therapeutic effect, such as for example, reduced number of cancer cells, reduced tumor size, reduced rate of cancer cell infiltration into peripheral organs, or reduced rate of tumor metastasis or tumor growth. “Treatment” may include one or more of the following: inducing/increasing an antitumor immune response, decreasing the number of one or more tumor markers, halting or delaying the growth of a tumor or blood cancer or progression of disease associated with ILT-3 or, when administered in combination with the anti-PD-1 antigen binding protein or antigen binding fragment, PD-1 binding to its ligands PD-L1 and/or PD-L2 (“PD-1-related disease”) such as cancer, stabilization of ILT-3-related disease, or PD-1-related disease (when administered in combination with the anti-PD-1 antigen binding protein or antigen binding fragment), inhibiting the growth or survival of tumor cells, eliminating or reducing the size of one or more cancerous lesions or tumors, decreasing the level of one or more tumor markers, ameliorating or abrogating the clinical manifestations of ILT-3 or PD-1-related disease (when administered in combination with the anti-PD-1 antigen binding protein or antigen binding fragment), reducing the severity or duration of the clinical symptoms of ILT-3- or PD-1-(when administered in combination with the anti-PD-1 antigen binding protein or antigen binding fragment) related disease such as cancer, prolonging the survival of a patient relative to the expected survival in a similar untreated patient, and inducing complete or partial remission of a cancerous condition or other ILT-3 or PD-1 (when administered in combination with the anti-PD-1 antigen binding protein or antigen binding fragment) related disease.

Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by a therapeutically effective amount is any of progression free survival (PFS), disease free survival (DFS) or overall survival (OS). PFS, also referred to as “Time to Tumor Progression” indicates the length of time during and after treatment that the cancer does not grow and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients. While an embodiment of the methods, compositions and uses of the present invention may not be effective in achieving a positive therapeutic effect in every patient, it should do so in a statistically significant number of subjects as determined by any statistical test known in the art such as the Student's t-test, the chi2-test, the U-test according to Mann and Whitney, the Kruskal-Wallis test (H-test), Jonckheere-Terpstra-test and the Wilcoxon-test.

The terms “effective amount”, “therapeutically effective amount”, and “therapeutically effective dose” refer to an amount of an anti-ILT3 antigen binding protein or antigen binding fragment (e.g., an anti-ILT3 antibody), and/or an anti-PD1 antigen binding protein or antigen binding fragment (e.g., an anti-PD1 antibody such as pembrolizumab) of the invention that, when administered alone or in combination with an additional therapeutic/prophylactic agent to a cell, tissue, or subject, is effective to prevent or cause a measurable improvement in one or more symptoms of disease or condition associated with the disease or condition being treated, e.g., whether that be cancer, mTNBC, GBM, mPDAC, mSTS, or mNSCLC as disclosed herein. An effective dose further refers to that amount of the anti-ILT3 antigen binding protein or antigen binding fragment or anti-PD1 antigen binding protein or antigen binding fragment sufficient to result in at least partial prevention or amelioration of symptoms of the disease or condition being treated, either alone or in combination with another compound. When applied to an individual active ingredient administered alone, an effective dose refers to that ingredient alone. When applied to a combination, a therapeutically effective amount refers to combined amounts of the active ingredients that result in the prophylactic or therapeutic effect, whether administered in combination, serially, or simultaneously.

The antigen binding proteins or antigen binding proteins disclosed herein may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound or compounds disclosed herein depend on the pharmacokinetic properties of that compound or compounds, such as absorption, distribution and half-life which can be determined by a skilled artisan. In addition, suitable dosing regimens, including the duration such regimens are administered, for a compound or compounds disclosed herein depend on the disease or condition being treated, the severity of the disease or condition, the age and physical condition of the subject being treated, the medical history of the subject being treated, the nature of concurrent therapy, the desired therapeutic effect, and like factors within the knowledge and expertise of the skilled artisan. It will be further understood by such skilled artisans that suitable dosing regimens may require adjustment given an individual subject's response to the dosing regimen or over time as the individual subject needs change. Typical daily dosages may vary depending upon the particular route of administration chosen.

The term “subject” (alternatively referred to as “patient” or “individual” herein) refers to a mammal (e.g., rat, mouse, dog, cat, rabbit) capable of being treated with the methods and compositions of the invention, most preferably a human. In some embodiments, the subject is an adult subject. In other embodiments, the subject is a pediatric subject.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytotoxic/antitumor antibiotics, topoisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, anti-sense oligonucleotides that that inhibit expression of genes implicated in abnormal cell proliferation or tumor growth. Chemotherapeutic agents useful in the methods of the present invention include cytostatic and/or cytotoxic agents. “Chemotherapy” refers to a cancer treatment using chemotherapeutic agents.

“Biologic agent” or “biotherapeutic agent” means a biological molecule, such as an antibody or fusion protein, that blocks ligand/receptor signaling in any biological pathway that supports tumor maintenance and/or growth or suppresses the anti-tumor immune response. “Biologic therapy” or “biological therapy” refers to a cancer treatment using a protein.

“Targeted agent” or “targeted therapeutic agent” refers to a therapeutic agent (either a small molecule or protein) that affects a specific protein type or class of proteins that are associated with tumor cell growth or spread in a patient's body.

“Systemic therapy” refers to a cancer treatment using therapeutic agents injected in a patient's bloodstream that affect cells throughout the patient's body, including chemotherapy, biological therapy, and targeted therapy.

“Platinum-containing chemotherapy” (also known as platins) refers to the use of chemotherapeutic agent(s) used to treat cancer that are coordination complexes of platinum. Platinum-containing chemotherapeutic agents are alkylating agents that crosslink DNA, resulting in ineffective DNA mismatch repair and generally leading to apoptosis. Examples of platins include cisplatin, carboplatin, and oxaliplatin.

The term “triple negative breast cancer” (TNBC) as used herein refers to a cancer that tests negative for estrogen receptors, progesterone receptors, and HER2.

The term “glioblastoma” (GBM) as used herein refers to cancer of glial cells in neuronal tissue. Under the World Health Organization (WHO) classification of central nervous system tumors, GBM are grade IV diffuse gliomas.

The term “Karnofsky performance status” (KPS) refers to a classification of functional impairment in a patient. This can be used to compare effectiveness of different therapies and to assess the prognosis in individual patients. Lower Karnofsky scores indicate worse survival for most serious illnesses (see O'Toole and Golden, West J Med. 1991 October; 155(4):384-7.). Karnofsky status and grades are indicated in Table 1 below.

TABLE 1
Karnofsky Status and Grade

| Karnofsky Status | Karnofsky Grade (%) |
|---|---|
| Normal, No complaints | 100 |
| Able to carry on normal activities. | 90 |
| Minor signs or symptoms of disease | |
| Normal activity with effort | 80 |
| Care for self. Unable to carry on | 70 |
| normal activity or to do active work | |
| Requires occasional assistance, but | 60 |
| able to care for most of his/her needs | |
| Requires considerable assistance | 50 |
| and frequent medical care | |
| Disabled. Requires special care | 40 |
| and assistance | |
| Severely disabled. Hospitalisation | 30 |
| indicated though death nonimminent | |
| Very sick. Hospitalisation necessary. | 20 |
| Active supportive treatment necessary | |
| Moribund | 10 |
| Dead | 0 |

The term “pancreatic ductal adenocarcinoma” (PDAC) refers to exocrine cell growth in ducts of the pancreas (see Haeberle, Lena, and Irene Esposito. “Pathology of pancreatic cancer.” Translational gastroenterology and hepatology vol. 4 50. 27 Jun. 2019).

The term “soft tissue sarcoma” (STS) refers to a malignant tumor of the soft tissue, such as fat, muscle, nerves, fibrous tissues, blood vessels, or deep skin tissues.

The term “non-squamous non-small cell lung carcinoma” (non-squamous NSCLC) refers to a non-small cell lung carcinoma that is non-squamous, and includes large-cell carcinoma, and adenocarcinoma. Non-squamous NSCLC accounts for about 50% of all NSCLC.

Cancer is staged for a given patient by combining Tumor score (T plus a number 0 to 4 describing the size and location of the tumor, and how much the tumor has grown into nearby tissues), Node score (N plus a number 0 to 3; often the number of lymph nodes with cancer), and Metastasis score (M plus a number 0 or 1; M1 indicates that the cancer has metastasized), as well as other factors specific to the particular cancer. Stage 0 describes cancer in situ, i.e., cancers still located in the tissue where they started and have not spread to nearby tissues. This stage of cancer is often highly curable, usually by removing the entire tumor with surgery. Stage I is usually a small cancer or tumor that has not grown deeply into nearby tissues and has not spread to the lymph nodes or other parts of the body. Stage II and Stage III indicate larger cancers or tumors that have grown more deeply into nearby tissue and may have spread to lymph nodes but not to other parts of the body. Stage IV means that the cancer has spread to other organs or parts of the body. It may also be called advanced or metastatic cancer.

### Immune Responses to Tumor Cells

Regulatory T cells play an important role in the maintenance of immunological self-tolerance by suppressing immune responses against autoimmune diseases and cancer. Accordingly, in one embodiment, upmodulating an immune response would be beneficial for enhancing an immune response in cancer. Therefore, the anti-ILT3 antigen binding proteins or antigen binding fragments disclosed herein may be used in the treatment of malignancies, to inhibit tumor growth or metastasis. The anti-ILT3 antigen binding proteins or antigen binding fragments disclosed herein may be administered systemically or locally to the tumor site

In one embodiment, modulation of human ILT3 function may be useful in the induction of tumor immunity. An anti-ILT3 antigen binding protein may be administered to a patient having tumor cells (e.g., sarcoma, melanoma, lymphoma, leukemia, neuroblastoma, carcinoma) to overcome tumor-specific tolerance in the subject.

As used herein, the term “neoplastic disease” is characterized by malignant tumor growth or in disease states characterized by benign hyperproliferative and hyperplastic cells. The common medical meaning of the term “neoplasia” refers to “new cell growth” that results as a loss of responsiveness to normal growth controls, e.g., neoplastic cell growth.

As used herein, the terms “hyperproliferative”, “hyperplastic”, malignant” and “neoplastic” are used interchangeably, and refer to those cells in an abnormal state or condition characterized by rapid proliferation or neoplasia. The terms are meant to include all types of hyperproliferative growth, hyperplastic growth, cancerous growths or oncogenic processes, metastatic tissues or malignantly transformed cells, tissues, or organs, irrespective of histopathologic type or stage of invasiveness. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumors,” which may be either benign, premalignant or malignant.

The terms “neoplasia,” “hyperplasia,” and “tumor” are often commonly referred to as “cancer,” which is a general name for more than 100 disease that are characterized by uncontrolled, abnormal growth of cells.

In one embodiment, the cancer is selected from the group consisting of: gastrointestinal cancer, gastric cancer, pancreatic cancer, melanomas, breast cancer, lung cancer (e.g., NSCLC), head and neck cancer, bronchus cancer, colorectal cancer, colon cancer, rectal cancer, prostate cancer, pancreatic cancer, stomach cancer, ovarian cancer, urinary bladder cancer, brain or central nervous system cancer (e.g., GBM), peripheral nervous system cancer, esophageal cancer, cervical cancer, uterine or endometrial cancer, cancer of the oral cavity or pharynx, liver cancer, renal cancer, testicular cancer, biliary tract cancer, small bowel or appendix cancer, salivary gland cancer, thyroid gland cancer, adrenal gland cancer, soft tissue sarcoma, osteosarcoma, chondrosarcoma, and cancer of hematological tissues.

In one embodiment, the cancer is selected from the group consisting of: metastatic triple negative breast cancer (mTNBC); glioblastoma multiforme (GBM); metastatic pancreatic ductal adenocarcinoma (mPDAC); metastatic soft tissue sarcoma (mSTS); and metastatic non-squamous non-small cell lung carcinoma (mNSCLC). In one embodiment, the cancer is triple negative breast cancer (mTNBC). In one embodiment, the cancer is glioblastoma multiforme (GBM). In one embodiment, the cancer is metastatic pancreatic ductal adenocarcinoma (mPDAC). In one embodiment, the cancer is metastatic soft tissue sarcoma (mSTS). In one embodiment the cancer is metastatic non-squamous non-small cell lung carcinoma (mNSCLC).

### Antibodies

As used herein, the term “antigen binding protein” refers to a polypeptide or protein that binds to an antigen, e.g., ILT3 or PD-1 protein. An antigen binding protein includes, but is not limited to, a bivalent antibody tetramer (2H+2L), a monovalent antibody (H+L), a bispecific antibody that targets an antigen and another target, a Fab fragment, a Fab′ fragment, a F(ab′)₂ fragment, an Fv region, and an ScFv. Unless otherwise indicated, the antigen binding proteins herein bind to and inhibit the activity of ILT3 or PD-1.

The term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2^{nd} 15 ed. Raven Press, N. Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

The term “hypervariable region” refers to the amino acid residues of an antibody that are responsible for antigen-binding. The hypervariable region comprises amino acid residues from a “complementarity determining region” or “CDR” (i.e., CDR1.1, CDR1.2 and CDR1.3 in the light chain variable domain and CDRH1, CDRH2 and CDRH3 in the heavy chain variable domain). Sec Kabat et al. (1991) Sequences of Proteins of Immunological Interest, 5^{th} Ed. Public Health Service, National Institutes of Health, Bethesda, Md. (defining the CDR 35 regions of an antibody by sequence); see also Chothia and Lesk (1987) J. Mol. Biol. 196: 901-917 (defining the CDR regions of an antibody by structure). The term “framework” or “FR” residues refers to those variable domain residues other than the hypervariable region residues defined herein as CDR residues.

Unless otherwise indicated, an “antibody fragment” or “antigen binding fragment” refers to antigen binding fragments of antibodies, i.e., antibody fragments that retain the ability to specifically bind to the antigen bound by the full-length antibody, e.g., fragments that retain one or more CDR regions. Examples of antibody binding fragments include, but are not limited to, Fab, Fab′, F(ab′)2, and Fv fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, useful in the present invention will bind to the target protein with an affinity that is at least two-fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g., the amino acid sequence of a mature human PD-1 or human PD-L1 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“CDR” or “CDRs” means complementarity determining region(s) in an immunoglobulin variable region.

“Framework region” or “FR” as used herein means the immunoglobulin variable regions excluding the CDR regions.

“Isolated antibody” and “isolated antibody fragment” refers to the purification status and in such context means the named molecule is substantially free of other biological molecules such as nucleic acids, proteins, lipids, carbohydrates, or other material such as cellular 10 debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Variable regions” or “V region” as used herein means the segment of IgG chains which is variable in sequence between different antibodies. It extends to Kabat residue 109 in the light chain and 113 in the heavy chain.

A variant of a heavy chain variable region sequence or full-length heavy chain sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence or full-length light chain sequence is identical to the reference sequence except having up to five conservative amino acid substitutions in the framework region (i.e., outside of the CDRs), and preferably has less than four, three or two conservative amino acid substitution in the framework region.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in the art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

The VH and VL regions can be further subdivided into regions of hypervariability, termed complementarity determining regions (CDR), interspersed with regions that are more conserved, termed framework regions (FR). Each VH and VL is composed of three CDR regions and four FR regions, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

The constant regions of the antibodies may mediate the binding of the immunoglobulin to host tissues or factors, including various cells of the immune system (e.g., effector cells) and the first component (C1q) of the classical complement system. Typically, the numbering of the amino acids in the heavy chain constant domain begins with number 118, which is in accordance with the Eu numbering scheme. The Eu numbering scheme is based upon the amino acid sequence of human IgG1 (Eu), which has a constant domain that begins at amino acid position 118 of the amino acid sequence of the IgG1 described in Edelman et al., Proc. Natl. Acad. Sci. USA. 63: 78-85 (1969), and is shown for the IgG1, IgG2, IgG3, and IgG4 constant domains in Béranger, et al., Ibid.

The variable regions of the heavy and light chains contain a binding domain comprising the CDRs that interacts with an antigen. A number of methods are available in the art for defining CDR sequences of antibody variable domains (see Dondelinger et al., Frontiers in Immunol. 9: Article 2278 (2018)). The common numbering schemes include the following
Kabat numbering scheme is based on sequence variability and is the most commonly used (See Kabat et al. Sequences of Proteins of Immunological Interest, 5th Ed. Public Health Service, National Institutes of Health, Bethesda, Md. (1991) (defining the CDR regions of an antibody by sequence);
Chothia numbering scheme is based on the location of the structural loop region (See Chothia & Lesk J. Mol. Biol. 196: 901-917 (1987); Al-Lazikani et al., J. Mol. Biol. 273: 927-948 (1997));
AbM numbering scheme is a compromise between the two used by Oxford Molecular's AbM antibody modelling software (see Karu et al., ILAR Journal 37: 132-141 (1995);
Contact numbering scheme is based on an analysis of the available complex crystal structures (See www.bioinf.org.uk: Prof. Andrew C. R. Martin's Group; Abhinandan & Martin, Mol. Immunol. 45:3832-3839 (2008).
IMGT (ImMunoGeneTics) numbering scheme is a standardized numbering system for all the protein sequences of the immunoglobulin superfamily, including variable domains from antibody light and heavy chains as well as T cell receptor chains from different species and counts residues continuously from 1 to 128 based on the germ-line V sequence alignment (see Giudicelli et al., Nucleic Acids Res. 25:206-11 (1997); Lefranc, Immunol Today 18:509(1997); Lefranc et al., Dev Comp Immunol. 27:55-77 (2003)).

The following general rules disclosed in www.bioinf.org.uk: Prof. Andrew C. R. Martin's Group and reproduced in Table 3 below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

TABLE 3
Antibody CDR Rules

| Loop | Kabat | AbM | Chothia¹ | Contact² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L51 |
| L3 | L89--L97 | L89--L97 | L89--L97 | L89--L96 | L89--L97 |
| H1 | H31-- | H26-- | H26--H32 | H30-- | H26-- |
| | H35B | H35B | to.34 | H35B | H35B |
| | (Kabat | | | | |
| | Numbering)³ | | | | |
| H1 | H31--H35 | H26--H35 | H26--H32 | H30--H35 | H26--H33 |
| | (Chothia | | | | |
| | Numbering) | | | | |
| H2 | H50--H65 | H50--H58 | H52--H56 | H47--H58 | H51--H56 |
| H3 | H95-- | H95-- | H95-- | H93-- | H93-- |
| | H102 | H102 | H102 | H101 | H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

In general, the state of the art recognizes that in many cases, the CDR3 region of the heavy chain is the primary determinant of antibody specificity, and examples of specific antibody generation based on CDR3 of the heavy chain alone are known in the art (e.g., Beiboer et al., J. Mol. Biol. 296: 833-849 (2000); Klimka et al., British J. Cancer 83: 252-260 (2000); Rader et al., Proc. Natl. Acad. Sci. USA 95: 8910-8915 (1998); Xu et al., Immunity 13: 37-45 (2000).

### Diagnostic Anti-PD-L Antibodies

“Diagnostic anti-PD-L monoclonal antibody” means a mAb which specifically binds to the mature form of the designated PD-L (PD-L1 or PD-L2) that is expressed on the surface of certain mammalian cells. A mature PD-L lacks the presecretory leader sequence, also referred to as leader peptide. The terms “PD-L” and “mature PD-L” are used interchangeably herein and shall be understood to mean the same molecule unless otherwise indicated or readily apparent from the context.

As used herein, a diagnostic anti-human PD-L1 mAb or an anti-hPD-L1 mAb refers to a monoclonal antibody that specifically binds to mature human PD-L1. A mature human PD-L1 molecule consists of amino acids 19-290 of the following sequence:

(SEQ ID NO: 1)
MRIFAVFIFMTYWHLLNAFTVTVPKDLYVVEYGSNMTIECKFPVE
KQLDLAALIVYWEMEDKNIIQFVHGEEDLKVQHSSYRQRARLLKD
QLSLGNAALQITDVKLQDAGVYRCMISYGGADYKRITVKVNAPYN
KINQRILVVDPVTSEHELTCQAEGYPKAEVIWTSSDHQVLSGKTT
TTNSKREEKLFNVTSTLRINTTTNEIFYCTFRRLDPEENHTAELV
IPELPLAHPPNERTHLVILGAILLCLGVALTFIFRLRKGRMMDVK
KCGIQDTNSKKQSDTHLEET.

Specific examples of diagnostic anti-human PD-L1 mAbs useful as diagnostic mAbs for immunohistochemistry (IHC) detection of PD-L1 expression in formalin-fixed, paraffin-embedded (FFPE) tumor tissue sections are antibody 20C3 and antibody 22C3, which are described in WO 2014/100079. These antibodies comprise the light chain and heavy chain variable region amino acid sequences shown in Table 4 below:

TABLE 4
Monoclonal Antibodies 20C3 and 22C3

| | |
|---|---|
| 20C3 Light Chain Mature | |
| Variable Region | |
| DIVMSQSPSSLAVSAGEKVTMSCKSSQSLL | SEQ ID NO: 2 |
| NSRTRKNYLAWYQQKPGQSPKLLIYWASTR | |
| ESGVPDRFTGSGSGTDFTLTISSVQAEDLA | |
| VYYCQQSYDVVTFGAGTKLELK | |
| 20C3 Heavy Chain Mature | |
| Variable Region | |
| QVQVQQSGAELAEPGASVKMSCKASGYIFT | SEQ ID NO: 3 |
| SYWMHWLKQRPGQGLEWIGYINPSSDYNEY | |
| SEKFMDKATLTADKASTTAYMQLISLTSED | |
| SAVYYCARSGWLVHGDYYFDYWGQGTTLTV | |
| SS | |
| 22C3 Light Chain Mature | |
| Variable Region | |
| DIVMSQSPSSLAVSAGEKVTMTCKSSQSLL | SEQ ID NO: 4 |
| HTSTRKNYLAWYQQKPGQSPKLLIYWASTR | |
| ESGVPDRFTGSGSGTDFTLTISSVQAEDLA | |
| VYYCKQSYDVVTFGAGTKLELK | |
| 22C3 Heavy Chain Mature | |
| Variable Region | |
| QVHLQQSGAELAKPGASVKMSCKASGYTFT | SEQ ID NO: 5 |
| SYWIHWIKQRPGQGLEWIGYINPSSGYHEY | |
| NQKFIDKATLTADRSSSTAYMHLTSLTSED | |
| SAVYYCARSGWLIHGDYYFDFWGQGTTLTV | |
| SS | |

Another anti-human PD-L1 mAb that has been reported to be useful for IHC detection of PD-L1 expression in FFPE tissue sections (Chen, B. J. et al., Clin Cancer Res 19:3462-3473 (2013)) is a rabbit anti-human PD-L1 mAb publicly available from Sino Biological, Inc. (Beijing, P.R. China; Catalog number 10084-R015).

### PD-L1 and PD-L2 Tissue Expression

“PD-L1” or “PD-L2” expression means any detectable level of expression of the designated PD-L protein on the cell surface or of the designated PD-L mRNA within a cell or tissue, unless otherwise defined. PD-L protein expression may be detected with a diagnostic PD-L antibody in an IHC assay of a tumor tissue section or by flow cytometry. Alternatively, PD-L protein expression by tumor cells may be detected by PET imaging, using a binding agent (e.g., antibody fragment, affibody and the like) that specifically binds to the desired PD-L target, e.g., PD-L1 or PD-L2. Techniques for detecting and measuring PD-L mRNA expression include RT-PCR and real-time quantitative RT-PCR.

Several approaches have been described for quantifying PD-L1 protein expression in IHC assays of tumor tissue sections. See, e.g., Thompson et al., PNAS 101 (49): 17174-17179 (2004); Thompson et al., Cancer Res. 66:3381-3385 (2006); Gadiot et al., Cancer 117:2192-2201 (2011); Taube et al., Sci Transl Med 4, 127ra37 (2012); and Toplian et al., New Eng. J Med. 366 (26): 2443-2454 (2012).

One approach employs a simple binary endpoint of positive or negative for PD-L1 expression, with a positive result defined in terms of the percentage of tumor cells that exhibit histologic evidence of cell-surface membrane staining. A tumor tissue section is counted as positive for PD-L1 expression is at least 1%, and preferably 5% of total tumor cells.

In another approach, PD-L1 expression in the tumor tissue section is quantified in the tumor cells as well as in infiltrating immune cells, which predominantly comprise lymphocytes. The percentage of tumor cells and infiltrating immune cells that exhibit membrane staining are separately quantified as <5%, 5 to 9%, and then in 10% increments up to 100%. For tumor cells, PD-L1 expression is counted as negative if the score is <5% score and positive if the score is ≥5%. PD-L1 expression in the immune infiltrate is reported as a semi-quantitative measurement called the adjusted inflammation score (AIS), which is determined by multiplying the percent of membrane staining cells by the intensity of the infiltrate, which is graded as none (0), mild (score of 1, rare lymphocytes), moderate (score of 2, focal infiltration of tumor by lymphohistiocytic aggregates), or severe (score of 3, diffuse infiltration). A tumor tissue section is counted as positive for PD-L1 expression by immune infiltrates if the AIS is ≥5.

A tissue section from a tumor that has been stained by IHC with a diagnostic PD-L1 antibody may also be scored for PD-L1 protein expression by assessing PD-L1 expression in both the tumor cells and infiltrating immune cells in the tissue section using a scoring process. See WO 2014/165422. One PD-L1 scoring process comprises examining each tumor nest in the tissue section for staining and assigning to the tissue section one or both of a modified H score (MHS) and a modified proportion score (MPS). To assign the MHS, four separate percentages are estimated across all of the viable tumor cells and stained mononuclear inflammatory cells in all of the examined tumor nests: (a) cells that have no staining (intensity=0), (b) weak staining (intensity=1+), (c) moderate staining (intensity=2+) and (d) strong staining (intensity=3+). A cell must have at least partial membrane staining to be included in the weak, moderate or strong staining percentages. The estimated percentages, the sum of which is 100%, are then input into the formula of 1× (percent of weak staining cells)+2× (percent of moderate staining cells)+3× (percent of strong staining cells), and the result is assigned to the tissue section as the MHS. The MPS is assigned by estimating, across all of the viable tumor cells and stained mononuclear inflammatory cells in all of the examined tumor nests, the percentage of cells that have at least partial membrane staining of any intensity, and the resulting percentage is assigned to the tissue section as the MPS. In some embodiments, the tumor is designated as positive for PD-L1 expression if the MHS or the MPS is positive.

Another method for scoring/quantifying PD-L1 expression in a tumor is the “combined positive score” or “CPS,” which refers to an algorithm for determining a PD-L1 expression score from a tumor sample of a patient. The CPS is useful in selecting patients for treatment with particular treatment regimens including methods of treatment comprising administration of an anti-PD-1 antigen binding protein or antigen binding fragment in which expression of PD-L1 is associated with a higher response rate in a particular patient population relative to same patient population that does not express PD-L1. The CPS is determined by determining the number of viable PD-L1 positive tumor cells, the number of viable PD-L1 negative tumor cells, and the number of viable PD-L1 positive mononuclear inflammatory cells (MIC) in a tumor tissue from a patient having a tumor and calculating the CPS using the following formula:

(#PD-L1 positive tumor cells)+(#PD-L1 positive MIC)×100%

(#PD-L1 positive tumor cells)+(PD-L1 negative tumor cells).

Yet another scoring method for PD-L1 expression is the “TPS” or “tumor proportion score,” which is the percentage of tumor cells expressing PD-L1 on the cell membrane. TPS typically includes the percentage of neoplastic cells expressing PD-L1 at any intensity (weak, moderate, or strong), which can be determining using an immunohistochemical assay using a diagnostic anti-human PD-L1 mAb, e.g., antibody 20C3 and antibody 22C3, described above. Cells are considered to express PD-L1 if membrane staining is present, including cells with partial membrane staining.

The level of PD-L1 mRNA expression may be compared to the mRNA expression levels of one or more reference genes that are frequently used in quantitative RT-PCR, such as ubiquitin C.

In some embodiments, a level of PD-L1 expression (protein and/or mRNA) by malignant cells and/or by infiltrating immune cells within a tumor is determined to be “overexpressed” or “elevated” based on comparison with the level of PD-L1 expression (protein and/or mRNA) by an appropriate control. For example, a control PD-L1 protein or mRNA expression level may be the level quantified in nonmalignant cells of the same type or in a section from a matched normal tissue. In some preferred embodiments, PD-L1 expression in a tumor sample is determined to be elevated if PD-L1 protein (and/or PD-L1 mRNA) in the sample is at least 10%, 20%, or 30% greater than in the control.

“Tissue section” refers to a single part or piece of a tissue sample, e.g., a thin slice of tissue cut from a sample of a normal tissue or of a tumor.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“RECIST 1.1 Response Criteria” as used herein means the definitions set forth in Eisenhauer, E. A. et al., Eur. J. Cancer 45:228-247 (2009) for target lesions or non-target lesions, as appropriate based on the context in which response is being measured.

### Anti-ILT3 Antibodies and Antigen Binding Fragments Useful in the Invention

An “anti-ILT3 antigen binding protein or antigen binding fragment” useful in the any of the methods, compositions and uses of the present invention include monoclonal antibodies (mAb), or antigen binding fragments thereof, which specifically bind to human ILT3. Alternative names or synonyms for ILT3 include: LILRB4; LIR5; and CD85K. In any of the methods, compositions and uses of the present invention in which a human individual is being treated, the anti-ILT3 antigen binding protein, antibody or antigen binding fragment binds to ILT3 and reduces the ability of MDSCs to suppress T-cell activation and proliferation. An anti-ILT3 antibody may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

The term “anti-ILT3 antigen binding protein” refers to a protein that binds the extracellular domain (amino acids 22-259) of GenPept Acc. No. Q8NHJ6.3:

(SEQ ID NO: 6)
QAGPLPKPTLWAEPGSVISWGNSVTIWCQGTLEAREYRLDKEESP
APWDRQNPLEPKNKARFSIPSMTEDYAGRYRCYYRSPVGWSQPSD
PLELVMTGAYSKPTLSALPSPLVTSGKSVTLLCQSRSPMDTFLLI
KERAAHPLLHLRSEHGAQQHQAEFPMSPVTSVHGGTYRCFSSHGF
SHYLLSHPSDPLELIVSGSLEDPRPSPTRSVSTAAGPEDQPLMPT
GSVPHSGLRRHWE

Examples of m Abs that bind to human ILT3, useful in the methods and uses of the invention are described in WO2019/099597 (incorporated by reference herein) and summarized below in Table 5.

TABLE 5
Exemplary anti-ILT3 antibody regions and constant regions

| SEQ | | |
|---|---|---|
| ID | | |
| NO: | Description | Sequence |
| 7 | Human IgG4 HC | ASTKGPSVFPLAPCSRSTSESTAALGCLVKDYFPEPVTV |
| | Constant domain | SWNSGALTSGVHTFPAVLQSSGLYSLSSVVTVPSSSLG |
| | Residue 108 | TKTYTCNVDHKPSNTKVDKRVESKYGPPCPPCPAPEFL |
| | corresponds with | GGPSVFLFPPKPKDTLMISRTPEVTCVVVDVSQEDPEV |
| | S228P | QFNWYVDGVEVHNAKTKPREEQFNSTYRVVSVLTVL |
| | | HQDWLNGKEYKCKVSNKGLPSSIEKTISKAKGQPREP |
| | | QVYTLPPSQEEMTKNQVSLTCLVKGFYPSDIAVEWES |
| | | NGQPENNYKTTPPVLDSDGSFFLYSRLTVDKSRWQEG |
| | | NVFSCSVMHEALHNHYTQKSLSLSLGK |
| 8 | Human IgG4 HC | ASTKGPSVFPLAPCSRSTSESTAALGCLVKDYFPEPVTV |
| | Constant domain | SWNSGALTSGVHTFPAVLQSSGLYSLSSVVTVPSSSLG |
| | Residue 108 | TKTYTCNVDHKPSNTKVDKRVESKYGPPCPPCPAPEFL |
| | corresponds with | GGPSVFLFPPKPKDTLMISRTPEVTCVVVDVSQEDPEV |
| | S228P | QFNWYVDGVEVHNAKTKPREEQFNSTYRVVSVLTVL |
| | (lacks C-terminal | HQDWLNGKEYKCKVSNKGLPSSIEKTISKAKGQPREP |
| | K, (herein referred | QVYTLPPSQEEMTKNQVSLTCLVKGFYPSDIAVEWES |
| | to as “K-”) | NGQPENNYKTTPPVLDSDGSFFLYSRLTVDKSRWQEG |
| | | NVFSCSVMHEALHNHYTQKSLSLSLG |
| 9 | Human IgG1 HC | ASTKGPSVFPLAPSSKSTSGGTAALGCLVKDYFPEPVT |
| | constant domain | VSWNSGALTSGVHTFPAVLQSSGLYSLSSVVTVPSSSL |
| | | GTQTYICNVNHKPSNTKVDKKVEPKSCDKTHTCPPCP |
| | | APELLGGPSVFLFPPKPKDTLMISRTPEVTCVVVDVSH |
| | | EDPEVKFNWYVDGVEVHNAKTKPREEQYNSTYRVVS |
| | | VLTVLHQDWLNGKEYKCKVSNKALPAPIEKTISKAKG |
| | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| 10 | Human IgG1 HC | ASTKGPSVFPLAPSSKSTSGGTAALGCLVKDYFPEPVT |
| | Constant domain | VSWNSGALTSGVHTFPAVLQSSGLYSLSSVVTVPSSSL |
| | Residue 117 | GTQTYICNVNHKPSNTKVDKKVEPKSCDKTHTCPPCP |
| | corresponds with | APEAAGGPSVFLFPPKPKDTLMISRTPEVTCVVVSVSH |
| | L234A, residue | EDPEVKFNWYVDGVEVHNAKTKPREEQYNSTYRVVS |
| | 118 corresponds | VLTVLHQDWLNGKEYKCKVSNKALPAPIEKTISKAKG |
| | with L235A, | QPREPQVYTLPPSRDELTKNQVSLTCLVKGFYPSDIAV |
| | residue 148 | EWESNGQPENNYKTTPPVLDSDGSFFLYSKLTVDKSR |
| | corresponds with | WQQGNVFSCSVMHEALHNHYTQKSLSLSPGK |
| | D265S | |
| 11 | Human IgG1 HC | ASTKGPSVFPLAPSSKSTSGGTAALGCLVKDYFPEPVT |
| | Constant domain | VSWNSGALTSGVHTFPAVLQSSGLYSLSSVVTVPSSSL |
| | (K-) Residue 117 | GTQTYICNVNHKPSNTKVDKKVEPKSCDKTHTCPPCP |
| | corresponds with | APEAAGGPSVFLFPPKPKDTLMISRTPEVTCVVVSVSH |
| | L234A, residue | EDPEVKFNWYVDGVEVHNAKTKPREEQYNSTYRVVS |
| | 118 corresponds | VLTVLHQDWLNGKEYKCKVSNKALPAPIEKTISKAKG |
| | with L235A, | QPREPQVYTLPPSRDELTKNQVSLTCLVKGFYPSDIAV |
| | residue 148 | EWESNGQPENNYKTTPPVLDSDGSFFLYSKLTVDKSR |
| | corresponds with | WQQGNVFSCSVMHEALHNHYTQKSLSLSPG |
| | D265S | |
| 12 | Human LC Kappa | RTVAAPSVFIFPPSDEQLKSGTASVVCLLNNFYPREAK |
| | Constant domain | VQWKVDNALQSGNSQESVTEQDSKDSTYSLSSTLTLS |
| | | KADYEKHKVYACEVTHQGLSSPVTKSFNRGEC |
| 13 | Anti-ILT3 52B8 | EVQLVESGGDLVKPGGSLKLSCAASGFTFSNYGMSWV |
| | parental HC | RQTPDRRLEWVATISGGGDYTNYPDSMRGRFTISRDN |
| | variable domain | AKNTLYLQMSSLKSEDTAMYYCGRRLWFRSLYYAMD |
| | | YWGQGTSVTVSS |
| 14 | Anti-ILT3 52B8 | NIVLTQSPASLAVSLGQRATISCRASEKVDSFGNSFMH |
| | parental LC | WYQQKPGQPPKLLIYLTSNLDSGVPARFSGSGSRTDFA |
| | variable domain | LTIDPVEADDAATYYCQQNNEDPYTFGGGTKLEIK |
| 15 | 52B8 HC-CDR1 | NYGMS |
| 16 | 52B8 HC-CDR2 | TISGGGDYTNYPDSXRG |
| | (Wherein Xaa15 is | |
| | M, V, or L) | |
| 17 | 52B8 HC-CDR2 | TISGGGDYTNYPDSMRG |
| | M | |
| 18 | 52B8 HC-CDR2 V | TISGGGDYTNYPDSVRG |
| 19 | 52B8 HC-CDR2 L | TISGGGDYTNYPDSLRG |
| 20 | 52B8 HC-CDR3 | RLXFRSLYYAMDY |
| | (Wherein Xaa3 is | |
| | W, Y, Q, or F) | |
| 21 | 52B8 HC-CDR3 | RLWFRSLYYAMDY |
| 22 | 52B8 HC-CDR3 | RLYFRSLYYAMDY |
| 23 | 52B8 HC-CDR3 | RLQFRSLYYAMDY |
| 24 | 52B8 HC-CDR3 | RLFFRSLYYAMDY |
| 25 | 52B8 LC-CDR1 | RASEKVDSFGXXFMH |
| | (Wherein Xaa11 is | |
| | N, D, or Q and | |
| | Xaa12 is S, N, or | |
| | A) | |
| 26 | 52B8 LC-CDR1 N | RASEKVDSFGNXFMH |
| | (Wherein Xaa12 is | |
| | S, N, or A) | |
| 27 | 52B8 LC-CDR1 D | RASEKVDSFGDXFMH |
| | (Wherein Xaa12 is | |
| | S, N, or A) | |
| 28 | 52B8 LC-CDR1 Q | RASEKVDSFGQXFMH |
| | (Wherein Xaa12 is | |
| | S, N, or A) | |
| 29 | 52B8 LC-CDR1 S | RASEKVDSFGXSFMH |
| | (Wherein Xaa11 is | |
| | N, D, or Q) | |
| 30 | 52B8 LC-CDR1 N | RASEKVDSFGXNFMH |
| | (Wherein Xaa11 is | |
| | N, D, or Q) | |
| 31 | 52B8 LC-CDR1 A | RASEKVDSFGXAFMH |
| | (Wherein Xaa11 is | |
| | N, D, or Q) | |
| 32 | 52B8 LC-CDR1 | RASEKVDSFGNNFMH |
| | (NN) | |
| 33 | 52B8 LC-CDR1 | RASEKVDSFGDNFMH |
| | (DN) | |
| 34 | 52B8 LC-CDR1 | RASEKVDSFGQNFMH |
| | (QN) | |
| 35 | 52B8 LC-CDR1 | RASEKVDSFGNSFMH |
| | (NS) | |
| 36 | 52B8 LC-CDR1 | RASEKVDSFGDSFMH |
| | (DS) | |
| 37 | 52B8 LC-CDR1 | RASEKVDSFGNAFMH |
| | (NA) | |
| 38 | 52B8 LC-CDR1 | RASEKVDSFGDAFMH |
| | (DA) | |
| 39 | 52B8 LC-CDR1 | RASEKVDSFGQSFMH |
| | (QS) | |
| 40 | 52B8 LC-CDR1 | RASEKVDSFGQAFMH |
| | (AF) | |
| 41 | 52B8 LC-CDR2 | LTSNLDS |
| 42 | 52B8 LC-CDR3 | QQNNEDPYT |
| 43 | Anti-ILT3 40A6 | QVQLKESGPGLVQASETLSLTCTVSGFSLTSYSINWVR |
| | parental HC | QSSGKGPEWMGRFWYDEGIAYNLTLESRLSISGDTSK |
| | variable domain | NQVFLKMNSLRTGDTGTYYCTRDRDTVGITGWFAYW |
| | | GQGTLVTVSS |
| 44 | Anti-ILT3 40A6 | ETVMTQSPTSLSASIGERVTLNCKASQSVGVNVDWYQ |
| | parental LC | QTPGQSPKLLIYGSANRHTGVPDRFTGSGFGSDFTLT |
| | variable domain | ISDVEPEDLGVYYCLQYGSVPYTFGAGTKLELK |
| 45 | 40A6 HC-CDR1 | SYSIN |
| 46 | 40A6 HC-CDR2 | RFWYDEGIAYNLTLES |
| 47 | 40A6 HC-CDR3 | DRDTVGITGWFAY |
| 48 | 40A6 LC-CDR1 | KASQSVGVNVD |
| 49 | 40A6 LC-CDR2 | GSANRHT |
| 50 | 40A6 LC-CDR3 | LQYGSVPYT |
| 51 | Anti-ILT3 16B1 | QVQLKESGPGLVQASETLSLTCTVSGFSLTNYCVNWV |
| | parental HC | RQPSGKGPEWLGRFWFDEGKAYNLTLESRLSISGDTSK |
| | variable domain | NQVFLRMNSLRADDTGTYYCTRDRDTVGITGWFAYW |
| | | GQGTLVTVSS |
| 52 | Anti-ILT3 16B1 | ETVMTQSPTSLSASIGERVTLNCKASQSVGINVDWYQ |
| | parental LC | QTPGQSPKLLIYGSANRHTGVPDRFTGSGFGSDFTLT |
| | variable domain | ISNVEPEDLGVYYCLQYGSVPYTFGPGTKLELK |
| 53 | 16B1 HC-CDR1 | NYCVN |
| 54 | 16B1 HC-CDR2 | RFWFDEGKAYNLTLES |
| 55 | 16B1 HC-CDR3 | DRDTVGITGWFAY |
| 56 | 16B1 LC-CDR1 | KASQSVGINVD |
| 57 | 16B1 LC-CDR2 | GSANRHT |
| 58 | 16B1 LC-CDR3 | LQYGSVPYT |
| 59 | Anti-ILT3 11D1 | QVQLQQSGAELMKPGASVKISCKATGYTFRTYWIEWV |
| | parental HC | KQRPGHGLEWIGEILPGNGNTHENENFKDKATFTADTS |
| | variable domain | SNAAYMQLSSLTSEDSAVYYCVRRLGRGPFDFWGQG |
| | | TTLTVSS |
| 60 | Anti-ILT3 11D1 | DIQMTQSPSSLSVSLGGKVTITCKASQDINEYIGWYQR |
| | parental LC | KPGKGPRLLIHYTSTLQSGIPSRFSGSGSGRDYSLSIS |
| | variable domain | NLEPEDIATYYCLQYANPLPTFGGGTKLEIK |
| 61 | 11D1 HC-CDR1 | TYWIE |
| 62 | 11D1 HC-CDR2 | EILPGNGNTHENENFKD |
| 63 | 11D1 HC-CDR3 | RRLGRGPFDF |
| 64 | 11D1 LC-CDR1 | KASQDINEYIG |
| 65 | 11D1 LC-CDR2 | YTSTLQS |
| 66 | 11D1 LC-CDR3 | LQYANPLPT |
| 67 | Anti-ILT3 17H12 | EVQLVESGGGLVQPGRSMKLSCAASGFTFSNFDMAW |
| | parental HC | VRQAPTRGLEWVSSITYDGGSTSYRDSVKGRFTISRDN |
| | variable domain | AKGTLYLQMDSLRSEDTATYYCTTVESIATISTYFDYW |
| | | GQGVMVTVSS |
| 68 | Anti-ILT3 17H12 | DIVLTQSPALAVSLGQRATISCRASQSVSMSRYDLIHW |
| | parental LC | YQQKPGQQPKLLIFRASDLASGIPARFSGSGSGTDFTLT |
| | variable domain | INPVQADDIATYYCQQTRKSPPTFGGGTRLELK |
| 69 | 17H12 HC-CDR1 | NFDMA |
| 70 | 17H12 HC-CDR2 | SITYDGGSTSYRDSVKG |
| 71 | 17H12 HC-CDR3 | VESIATISTYFDY |
| 72 | 17H12 LC-CDR1 | RASQSVSMSRYDLIH |
| 73 | 17H12 LC-CDR2 | RASDLAS |
| 74 | 17H12 LC-CDR3 | QQTRKSPPT |
| 75 | Anti-ILT3 37C8 | QVQLKESGPGLVQASETLSLTCTVSGFSLTSYCVNWV |
| | parental HC | RQPSGKGPEWLGRFWYDEGKVYNLTLESRLSISGDTS |
| | variable domain | KNQVFLKMNRLRTDDTGTYYCTRDRDTMGITGWFAY |
| | | WGQGTLVTVSS |
| 76 | Anti-ILT3 37C8 | ETVMTQSPTSLSASIGERVTLNCKASQSVGINVDWYQ |
| | parental LC | QTPGQSPKLLIYGSANRHTGVPDRFTGSGFGSGFTLTIS |
| | variable domain | NVEPEDLGVYYCLQYGSVPYTFGPGTKLELK |
| 77 | 37C8 HC-CDR1 | SYCVN |
| 78 | 37C8 HC-CDR2 | RFWYDEGKVYNLTLES |
| 79 | 37C8 HC-CDR3 | DRDTMGITGWFAY |
| 80 | 37C8 LC-CDR1 | KASQSVGINVD |
| 81 | 37C8 LC-CDR2 | GSANRHT |
| 82 | 37C8 LC-CDR3 | LQYGSVPYT |
| 83 | Anti-ILT3 1G12 | QVQMQQSGTELMKPGASMKISCKATGYTFSTYWIQWI |
| | parental HC | KQRPGHGLEWIGEILPGSGTTNYNENFKGKATFSADTS |
| | variable domain | SNTAYIHLSSLTSEDSAVFYCARRLGRGPFDYWGQGTT |
| | | LTVSS |
| 84 | Anti-ILT3 1G12 | DIQMTQSPSSLSASLGGKVTITCEASQDINKHIDWYQH |
| | parental LC | QPGRGPSLLIHYASILQPGIPSRFSGSGSGRDYSFSIT |
| | variable domain | SLEPEDIATYYCLQYDNLLPTFGGGTKLEIK |
| 85 | 1G12 HC-CDR1 | TYWIQ |
| 86 | 1G12 HC-CDR2 | EILPGSGTTNYNENFKG |
| 87 | 1G12 HC-CDR3 | RLGRGPFDY |
| 88 | 1G12 LC-CDR1 | EASQDINKHID |
| 89 | 1G12 LC-CDR2 | YASILQP |
| 90 | 1G12 LC-CDR3 | LQYDNLLPT |
| 91 | Anti-ILT3 20E4 | QVQLKESGPGLVQASETLSLTCTVSGFSLTSYSVNWVR |
| | parental HC | QPSGKGLEWMGRFWYDGGTAYNSTLESRLSISGDTSK |
| | variable domain | NQVFLKMNSLQTDDTGTYYCTRDRDTMGITGWFAYW |
| | | GQGTLVTVSP |
| 92 | Anti-ILT3 20E4 | ETVMTQSPTSLSASIGERVTLNCKASQSVGVNVDWYQQ |
| | parental LC | TPGQSPKLLIYGSANRHTGVPDRFTGSGFGSDFTLTIS |
| | variable domain | NVEPEDLGVYYCLQYGSVPYTFGAGTKLELK |
| 93 | 20E4 HC-CDR1 | SYSVN |
| 94 | 20E4 HC-CDR2 | RFWYDGGTAYNSTLES |
| 95 | 20E4 HC-CDR3 | DRDTMGITGWFAY |
| 96 | 20E4 LC-CDR1 | KASQSVGVNVD |
| 97 | 20E4 LC-CDR2 | GSANRHT |
| 98 | 20E4 LC-CDR3 | LQYGSVPYT |
| 99 | Anti-ILT3 24A4 | QVQLKESGPGLVQASETLSLTCTVSGFSLTSYCVNWV |
| | parental HC | RQPSGKGPEWLGRFWYDEGKVYNLTLESRLSISGDTS |
| | variable domain | KNQVFLKMNRLRTDDTGTYYCTRDRDTLGITGWFAY |
| | | WGQGTLVTVSS |
| 100 | Anti-ILT3 24A4 | ETVMTQSPTSLSASIGERVTLNCKASQSVGINVDWYQ |
| | parental LC | QTPGQSPKLLIYGSANRHTGVPDRFTGSGFGSGFTLT |
| | variable domain | ISNVEPEDLGVYYCLQYGSVPYTFGPGTKLELK |
| 101 | 24A4 HC-CDR1 | SYCVN |
| 102 | 24A4 HC-CDR2 | RFWYDEGKVYNLTLES |
| 103 | 24A4 HC-CDR3 | DRDTLGITGWFAY |
| 104 | 24A4 LC-CDR1 | KASQSVGINVD |
| 105 | 24A4 LC-CDR2 | GSANRHT |
| 106 | 24A4 LC-CDR3 | LQYGSVPYT |
| 107 | Leader sequence A | MEWSWVFLFFLSVTTGVHS |
| 108 | Leader sequence B | MSVPTQVLGLLLLWLTDARC |
| 109 | Mouse Anti-ILT3 | EVQLVESGGDLVKPGGSLKLSCAASGFTFSNYGMSWV |
| | p52B8 parental | RQTPDRRLEWVATISGGGDYTNYPDSMRGRFTISRDN |
| | HC: Murine IgG2a | AKNTLYLQMSSLKSEDTAMYYCGRRLWFRSLYYAMD |
| | heavy chain | YWGQGTSVTVSSAKTTAPSVYPLAPVCGDTTGSSVTL |
| | | GCLVKGYFPEPVTLTWNSGSLSSGVHTFPAVLQSDLYT |
| | | LSSSVTVTSSTWPSQSITCNVAHPASSTKVDKKIEPRGP |
| | | TIKPCPPCKCPAPNLLGGPSVFIFPPKIKDVLMISLSPI |
| | | VTCVVVDVSEDDPDVQISWFVNNVEVHTAQTQTHREDY |
| | | NSTLRVVSALPIQHQDWMSGKEFKCKVNNKDLPAPIE |
| | | RTISKPKGSVRAPQVYVLPPPEEEMTKKQVTLTCMVT |
| | | DFMPEDIYVEWTNNGKTELNYKNTEPVLDSDGSYFM |
| | | YSKLRVEKKNWVERNSYSCSVVHEGLHNHHTTKSFSR |
| | | TPGK |
| 110 | Mouse Anti-ILT3 | NIVLTQSPASLAVSLGQRATISCRASEKVDSFGNSFMH |
| | p52B8 parental | WYQQKPGQPPKLLIYLTSNLDSGVPARFSGSGSRTDFA |
| | LC: murine Kappa | LTIDPVEADDAATYYCQQNNEDPYTFGGGTKLEIKRA |
| | light chain | DAAPTVSIFPPSSEQLTSGGASVVCFLNNFYPKDINVK |
| | | WKIDGSERQNGVLNSWTDQDSKDSTYSMSSTLTLTKD |
| | | EYERHNSYTCEATHKTSTSPIVKSFNRNEC |
| 111 | Chimeric Anti- | EVQLVESGGDLVKPGGSLKLSCAASGFTFSNYGMSWV |
| | ILT3 mouse 52B8 | RQTPDRRLEWVATISGGGDYTNYPDSMRGRFTISRDN |
| | VH | AKNTLYLQMSSLKSEDTAMYYCGRRLWFRSLYYAMD |
| | parental/human | YWGQGTSVTVSSASTKGPSVFPLAPCSRSTSESTAALG |
| | IgG4 (S228P) | CLVKDYFPEPVTVSWNSGALTSGVHTFPAVLQSSGLY |
| | | SLSSVVTVPSSSLGTKTYTCNVDHKPSNTKVDKRVESK |
| | | YGPPCPPCPAPEFLGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIE |
| | | KTISKAKGQPREPQVYTLPPSQEEMTKNQVSLTCLVKG |
| | | FYPSDIAVEWESNGQPENNYKTTPPVLDSDGSFFLYSR |
| | | LTVDKSRWQEGNVFSCSVMHEALHNHYTQKSLSLSLG |
| | | K |
| 112 | Chimeric Anti- | EVQLVESGGDLVKPGGSLKLSCAASGFTFSNYGMSWV |
| | ILT3 mouse 52B8 | RQTPDRRLEWVATISGGGDYTNYPDSVRGRFTISRDN |
| | VH M64V/human | AKNTLYLQMSSLKSEDTAMYYCGRRLWFRSLYYAMD |
| | IgG4 (S228P) | YWGQGTSVTVSSASTKGPSVFPLAPCSRSTSESTAALG |
| | | CLVKDYFPEPVTVSWNSGALTSGVHTFPAVLQSSGLY |
| | | SLSSVVTVPSSSLGTKTYTCNVDHKPSNTKVDKRVESK |
| | | YGPPCPPCPAPEFLGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIE |
| | | KTISKAKGQPREPQVYTLPPSQEEMTKNQVSLTCLVKG |
| | | FYPSDIAVEWESNGQPENNYKTTPPVLDSDGSFFLYSR |
| | | LTVDKSRWQEGNVFSCSVMHEALHNHYTQKSLSLSLG |
| | | K |
| 113 | Mouse Anti-ILT3 | EVQLVESGGDLVKPGGSLKLSCAASGFTFSNYGMSWV |
| | 52B8 VH | RQTPDRRLEWVATISGGGDYTNYPDSLRGRFTISRDNA |
| | M64L/human IgG4 | KNTLYLQMSSLKSEDTAMYYCGRRLWFRSLYYAMDY |
| | (S228P) | WGQGTSVTVSSASTKGPSVFPLAPCSRSTSESTAALGC |
| | | LVKDYFPEPVTVSWNSGALTSGVHTFPAVLQSSGLYSL |
| | | SSVVTVPSSSLGTKTYTCNVDHKPSNTKVDKRVESKY |
| | | GPPCPPCPAPEFLGGPSVFLFPPKPKDTLMISRTPEVTC |
| | | VVVDVSQEDPEVQFNWYVDGVEVHNAKTKPREEQFN |
| | | STYRVVSVLTVLHQDWLNGKEYKCKVSNKGLPSSIEK |
| | | TISKAKGQPREPQVYTLPPSQEEMTKNQVSLTCLVKGF |
| | | YPSDIAVEWESNGQPENNYKTTPPVLDSDGSFFLYSRL |
| | | TVDKSRWQEGNVFSCSVMHEALHNHYTQKSLSLSLG |
| | | K |
| 114 | Chimeric Anti- | NIVLTQSPASLAVSLGQRATISCRASEKVDSFGNSFMH |
| | ILT3 mouse 52B8 | WYQQKPGQPPKLLIYLTSNLDSGVPARFSGSGSRTDFA |
| | parental VL/ | LTIDPVEADDAATYYCQQNNEDPYTFGGGTKLEIKRT |
| | human Kappa | VAAPSVFIFPPSDEQLKSGTASVVCLLNNFYPREAKVQ |
| | | WKVDNALQSGNSQESVTEQDSKDSTYSLSSTLTLSKA |
| | | DYEKHKVYACEVTHQGLSSPVTKSFNRGEC |
| 115 | Humanized 52B8 | EVQLVESGGGLVQPGGSLRLSCAASGFTFSNYGMSWV |
| | HC variable | RQAPGKGLEWVATISGGGDYTNYPDSMRGRFTISRDN |
| | domain VH1 | AKNSLYLQMNSLRAEDTAVYYCGRRLWFRSLYYAMD |
| | | YWGQGTLVTVSS |
| 116 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLWFRSLYYAMD |
| | (M64V) | YWGQGTLVTVSS |
| 117 | Humanized 52B8 | EVQLVESGGGLVQPGGSLRLSCAASGFTFSNYGMSWV |
| | HC variable | RQAPGKGLEWVATISGGGDYTNYPDSLRGRFTISRDN |
| | domain VH1 | AKNSLYLQMNSLRAEDTAVYYCGRRLWFRSLYYAMD |
| | (M64L) | YWGQGTLVTVSS |
| 118 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLFFRSLYYAMD |
| | (M64V, W101F) | YWGQGTLVTVSS |
| 119 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLYFRSLYYAMD |
| | (M64V, W101Y) | YWGQGTLVTVSS |
| 120 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLQFRSLYYAMD |
| | (M64V, W101Q) | YWGQGTLVTVSS |
| 121 | Humanized 52B8 | EVQLVESGGGLVQPGGSLRLSCAASGFTFSNYGMSWV |
| | HC variable | RQAPGKGLEWVATISGGGDYTNYPDSMRGRFTISRDN |
| | domain VH2 | AKNSLYLQMNSLKAEDTAVYYCGRRLWFRSLYYAM |
| | | DYWGQGTLVTVSS |
| 122 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH2 | AKNSLYLQMNSLKAEDTAVYYCGRRLWFRSLYYAM |
| | (M64V) | DYWGQGTLVTVSS |
| 123 | Humanized 52B8 | EVQLVESGGGLVQPGGSLRLSCAASGFTFSNYGMSWV |
| | HC variable | RQAPGKGLEWVATISGGGDYTNYPDSLRGRFTISRDN |
| | domain VH2 | AKNSLYLQMNSLKAEDTAVYYCGRRLWFRSLYYAM |
| | (M64L) | DYWGQGTLVTVSS |
| 124 | Humanized 52B8 | DIVLTQSPDSLAVSLGERATINCRASEKVDSFGNSFMH |
| | LC variable | WYQQKPGQPPKLLIYLTSNLDSGVPDRFSGSGSRTDFT |
| | domain VL1 | LTISSLQAEDVAVYYCQQNNEDPYTFGQGTKLEIK |
| 125 | Humanized 52B8 | DIVLTQSPDSLAVSLGERATINCRASEKVDSFGNSFMH |
| | LC variable | WYQQKPGQPPKLLIYLTSNLDSGVPDRFSGSGSGTDFT |
| | domain VL2 | LTISSLQAEDVAVYYCQQNNEDPYTFGQGTKLEIK |
| 126 | Humanized 52B8 | EIVLTQSPATLSLSPGERATLSCRASEKVDSFGNSFMH |
| | LC variable | WYQQKPGQAPRLLIYLTSNLDSGVPARFSGSGSRTDFT |
| | domain VL3 | LTISSLEPEDFAVYYCQQNNEDPYTFGQGTKLEIK |
| 127 | Humanized 52B8 | EIVLTQSPATLSLSPGERATLSCRASEKVDSFGNSFMH |
| | LC variable | WYQQKPGQAPRLLIYLTSNLDSGIPARFSGSGSGTDFT |
| | domain VL4 | LTISSLEPEDFAVYYCQQNNEDPYTFGQGTKLEIK |
| 128 | Humanized 52B8 | DIQLTQSPSSLSASVGDRVTITCRASEKVDSFGNSFMH |
| | LC variable | WYQQKPGKAPKLLIYLTSNLDSGVPSRFSGSGSGTDFT |
| | domain VL5 | LTISSLQPEDFATYYCQQNNEDPYTFGQGTKLEIK |
| 129 | Humanized 52B8 | DIQMTQSPSSLSASVGDRVTITCRASEKVDSFGNSFMH |
| | LC variable | WYQQKPGKAPKLLIYLTSNLDSGVPSRFSGSGSRTDFT |
| | domain VL6 | LTISSLQPEDFATYYCQQNNEDPYTFGQGTKLEIK |
| 130 | Humanized 52B8 | DIQLTQSPSSLSASVGDRVTITCRASEKVDSFGNSFMH |
| | LC variable | WYQQKPGKAPKLLIYLTSNLDSGVPSRFSGSGSRTDFT |
| | domain VL7 | LTISSLQPEDFATYYCQQNNEDPYTFGQGTKLEIK |
| 131 | Humanized 52B8 | DIQLTQSPSSLSASVGDRVTITCRASEKVDSFGNSFMH |
| | LC variable | WYQQKPGKAPKLLIYLTSNLDSGVPARFSGSGSRTDFT |
| | domain VL8 | LTISSLQPEDFATYYCQQNNEDPYTFGQGTKLEIK |
| 132 | Humanized 52B8 | DIVLTQSPDSLAVSLGERATINCRASEKVDSFGNAFMH |
| | LC variable | WYQQKPGQPPKLLIYLTSNLDSGVPDRFSGSGSGTDFT |
| | domain VL2, | LTISSLQAEDVAVYYCQQNNEDPYTFGQGTKLEIK |
| | (S35A) | |
| 133 | Humanized 52B8 | DIVLTQSPDSLAVSLGERATINCRASEKVDSFGNNFMH |
| | LC variable | WYQQKPGQPPKLLIYLTSNLDSGVPDRFSGSGSGTDFT |
| | domain VL2, | LTISSLQAEDVAVYYCQQNNEDPYTFGQGTKLEIK |
| | (S35N) | |
| 134 | Humanized 52B8 | DIVLTQSPDSLAVSLGERATINCRASEKVDSFGQSFMH |
| | LC variable | WYQQKPGQPPKLLIYLTSNLDSGVPDRFSGSGSGTDFT |
| | domain VL2, | LTISSLQAEDVAVYYCQQNNEDPYTFGQGTKLEIK |
| | (N34Q) | |
| 135 | Humanized 52B8 | DIVLTQSPDSLAVSLGERATINCRASEKVDSFGDSFMH |
| | LC variable | WYQQKPGQPPKLLIYLTSNLDSGVPDRFSGSGSGTDFT |
| | domain VL2, | LTISSLQAEDVAVYYCQQNNEDPYTFGQGTKLEIK |
| | (N34D) | |
| 136 | Humanized 52B8 | DIQLTQSPSSLSASVGDRVTITCRASEKVDSFGNAFMH |
| | LC variable | WYQQKPGKAPKLLIYLTSNLDSGVPSRFSGSGSGTDFT |
| | domain VL5, | LTISSLQPEDFATYYCQQNNEDPYTFGQGTKLEIK |
| | (S35A) | |
| 137 | Humanized 52B8 | DIQLTQSPSSLSASVGDRVTITCRASEKVDSFGNNFMH |
| | LC variable | WYQQKPGKAPKLLIYLTSNLDSGVPSRFSGSGSGTDFT |
| | domain VL5, | LTISSLQPEDFATYYCQQNNEDPYTFGQGTKLEIK |
| | (S35N) | |
| 138 | Humanized 52B8 | DIQLTQSPSSLSASVGDRVTITCRASEKVDSFGQSFMH |
| | LC variable | WYQQKPGKAPKLLIYLTSNLDSGVPSRFSGSGSGTDFT |
| | domain VL5 | LTISSLQPEDFATYYCQQNNEDPYTFGQGTKLEIK |
| | (N34Q) | |
| 139 | Humanized 52B8 | DIQLTQSPSSLSASVGDRVTITCRASEKVDSFGDSFMH |
| | LC variable | WYQQKPGKAPKLLIYLTSNLDSGVPSRFSGSGSGTDFT |
| | domain VL5, | LTISSLQPEDFATYYCQQNNEDPYTFGQGTKLEIK |
| | (N34D) | |
| 140 | Humanized 52B8 | EVQLVESGGGLVQPGGSLRLSCAASGFTFSNYGMSWV |
| | HC variable | RQAPGKGLEWVATISGGGDYTNYPDSMRGRFTISRDN |
| | domain | AKNSLYLQMNSLRAEDTAVYYCGRRLWFRSLYYAMD |
| | VH1/Human IgG4 | YWGQGTLVTVSSASTKGPSVFPLAPCSRSTSESTAALG |
| | (S228P) constant | CLVKDYFPEPVTVSWNSGALTSGVHTFPAVLQSSGLY |
| | domain | SLSSVVTVPSSSLGTKTYTCNVDHKPSNTKVDKRVESK |
| | | YGPPCPPCPAPEFLGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIE |
| | | KTISKAKGQPREPQVYTLPPSQEEMTKNQVSLTCLVKG |
| | | FYPSDIAVEWESNGQPENNYKTTPPVLDSDGSFFLYSR |
| | | LTVDKSRWQEGNVFSCSVMHEALHNHYTQKSLSLSLG |
| | | K |
| 141 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLWFRSLYYAMD |
| | (M64V)/Human | YWGQGTLVTVSSASTKGPSVFPLAPCSRSTSESTAALG |
| | IgG4 (S228P) | CLVKDYFPEPVTVSWNSGALTSGVHTFPAVLQSSGLY |
| | constant domain | SLSSVVTVPSSSLGTKTYTCNVDHKPSNTKVDKRVESK |
| | | YGPPCPPCPAPEFLGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIE |
| | | KTISKAKGQPREPQVYTLPPSQEEMTKNQVSLTCLVKG |
| | | FYPSDIAVEWESNGQPENNYKTTPPVLDSDGSFFLYSR |
| | | LTVDKSRWQEGNVFSCSVMHEALHNHYTQKSLSLSLG |
| | | K |
| 142 | Humanized 52B8 | EVQLVESGGGLVQPGGSLRLSCAASGFTFSNYGMSWV |
| | HC variable | RQAPGKGLEWVATISGGGDYTNYPDSLRGRFTISRDN |
| | domain VH1 | AKNSLYLQMNSLRAEDTAVYYCGRRLWFRSLYYAMD |
| | (M64L)/Human | YWGQGTLVTVSSASTKGPSVFPLAPCSRSTSESTAALG |
| | IgG4 (S228P) | CLVKDYFPEPVTVSWNSGALTSGVHTFPAVLQSSGLY |
| | constant domain | SLSSVVTVPSSSLGTKTYTCNVDHKPSNTKVDKRVESK |
| | | YGPPCPPCPAPEFLGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIE |
| | | KTISKAKGQPREPQVYTLPPSQEEMTKNQVSLTCLVKG |
| | | FYPSDIAVEWESNGQPENNYKTTPPVLDSDGSFFLYSR |
| | | LTVDKSRWQEGNVFSCSVMHEALHNHYTQKSLSLSLG |
| | | K |
| 143 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLFFRSLYYAMD |
| | (M64V, | YWGQGTLVTVSSASTKGPSVFPLAPCSRSTSESTAALG |
| | W101F)/Human | CLVKDYFPEPVTVSWNSGALTSGVHTFPAVLQSSGLY |
| | IgG4 (S228P) | SLSSVVTVPSSSLGTKTYTCNVDHKPSNTKVDKRVESK |
| | constant domain | YGPPCPPCPAPEFLGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIE |
| | | KTISKAKGQPREPQVYTLPPSQEEMTKNQVSLTCLVKG |
| | | FYPSDIAVEWESNGQPENNYKTTPPVLDSDGSFFLYSR |
| | | LTVDKSRWQEGNVFSCSVMHEALHNHYTQKSLSLSLG |
| | | K |
| 144 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLYFRSLYYAMD |
| | (M64V, | YWGQGTLVTVSSASTKGPSVFPLAPCSRSTSESTAALG |
| | W101Y)/Human | CLVKDYFPEPVTVSWNSGALTSGVHTFPAVLQSSGLY |
| | IgG4 (S228P) | SLSSVVTVPSSSLGTKTYTCNVDHKPSNTKVDKRVESK |
| | constant domain | YGPPCPPCPAPEFLGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIE |
| | | KTISKAKGQPREPQVYTLPPSQEEMTKNQVSLTCLVKG |
| | | FYPSDIAVEWESNGQPENNYKTTPPVLDSDGSFFLYSR |
| | | LTVDKSRWQEGNVFSCSVMHEALHNHYTQKSLSLSLG |
| | | K |
| 145 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLQFRSLYYAMD |
| | (M64V, | YWGQGTLVTVSSASTKGPSVFPLAPCSRSTSESTAALG |
| | W101Q)/Human | CLVKDYFPEPVTVSWNSGALTSGVHTFPAVLQSSGLY |
| | IgG4 (S228P) | SLSSVVTVPSSSLGTKTYTCNVDHKPSNTKVDKRVESK |
| | constant domain | YGPPCPPCPAPEFLGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIE |
| | | KTISKAKGQPREPQVYTLPPSQEEMTKNQVSLTCLVKG |
| | | FYPSDIAVEWESNGQPENNYKTTPPVLDSDGSFFLYSR |
| | | LTVDKSRWQEGNVFSCSVMHEALHNHYTQKSLSLSLG |
| | | K |
| 146 | Humanized 52B8 | EVQLVESGGGLVQPGGSLRLSCAASGFTFSNYGMSWV |
| | HC variable | RQAPGKGLEWVATISGGGDYTNYPDSMRGRFTISRDN |
| | domain | AKNSLYLQMNSLKAEDTAVYYCGRRLWFRSLYYAM |
| | VH2/Human IgG4 | DYWGQGTLVTVSSASTKGPSVFPLAPCSRSTSESTAAL |
| | (S228P) constant | GCLVKDYFPEPVTVSWNSGALTSGVHTFPAVLQSSGL |
| | domain | YSLSSVVTVPSSSLGTKTYTCNVDHKPSNTKVDKRVES |
| | | KYGPPCPPCPAPEFLGGPSVFLFPPKPKDTLMISRTPEV |
| | | TCVVVDVSQEDPEVQFNWYVDGVEVHNAKTKPREEQ |
| | | FNSTYRVVSVLTVLHQDWLNGKEYKCKVSNKGLPSSI |
| | | EKTISKAKGQPREPQVYTLPPSQEEMTKNQVSLTCLVK |
| | | GFYPSDIAVEWESNGQPENNYKTTPPVLDSDGSFFLYS |
| | | RLTVDKSRWQEGNVFSCSVMHEALHNHYTQKSLSLSL |
| | | GK |
| 147 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH2 | AKNSLYLQMNSLKAEDTAVYYCGRRLWFRSLYYAM |
| | (M64V)/Human | DYWGQGTLVTVSSASTKGPSVFPLAPCSRSTSESTAAL |
| | IgG4 (S228P) | GCLVKDYFPEPVTVSWNSGALTSGVHTFPAVLQSSGL |
| | constant domain | YSLSSVVTVPSSSLGTKTYTCNVDHKPSNTKVDKRVES |
| | | KYGPPCPPCPAPEFLGGPSVFLFPPKPKDTLMISRTPEV |
| | | TCVVVDVSQEDPEVQFNWYVDGVEVHNAKTKPREEQ |
| | | FNSTYRVVSVLTVLHQDWLNGKEYKCKVSNKGLPSSI |
| | | EKTISKAKGQPREPQVYTLPPSQEEMTKNQVSLTCLVK |
| | | GFYPSDIAVEWESNGQPENNYKTTPPVLDSDGSFFLYS |
| | | RLTVDKSRWQEGNVFSCSVMHEALHNHYTQKSLSLSL |
| | | GK |
| 148 | Humanized 52B8 | EVQLVESGGGLVQPGGSLRLSCAASGFTFSNYGMSWV |
| | HC variable | RQAPGKGLEWVATISGGGDYTNYPDSLRGRFTISRDN |
| | domain VH2 | AKNSLYLQMNSLKAEDTAVYYCGRRLWFRSLYYAM |
| | (M64L)/Human | DYWGQGTLVTVSSASTKGPSVFPLAPCSRSTSESTAAL |
| | IgG4 (S228P) | GCLVKDYFPEPVTVSWNSGALTSGVHTFPAVLQSSGL |
| | constant domain | YSLSSVVTVPSSSLGTKTYTCNVDHKPSNTKVDKRVES |
| | | KYGPPCPPCPAPEFLGGPSVFLFPPKPKDTLMISRTPEV |
| | | TCVVVDVSQEDPEVQFNWYVDGVEVHNAKTKPREEQ |
| | | FNSTYRVVSVLTVLHQDWLNGKEYKCKVSNKGLPSSI |
| | | EKTISKAKGQPREPQVYTLPPSQEEMTKNQVSLTCLVK |
| | | GFYPSDIAVEWESNGQPENNYKTTPPVLDSDGSFFLYS |
| | | RLTVDKSRWQEGNVFSCSVMHEALHNHYTQKSLSLSL |
| | | GK |
| 149 | Humanized 52B8 | DIVLTQSPDSLAVSLGERATINCRASEKVDSFGNSFMH |
| | LC variable | WYQQKPGQPPKLLIYLTSNLDSGVPDRFSGSGSRTDFT |
| | domain VL1/kappa | LTISSLQAEDVAVYYCQQNNEDPYTFGQGTKLEIKRTV |
| | constant domain | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |
| 150 | Humanized 52B8 | DIVLTQSPDSLAVSLGERATINCRASEKVDSFGNSFMH |
| | LC variable | WYQQKPGQPPKLLIYLTSNLDSGVPDRFSGSGSGTDFT |
| | domain VL2/kappa | LTISSLQAEDVAVYYCQQNNEDPYTFGQGTKLEIKRTV |
| | constant domain | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |
| 151 | Humanized 52B8 | EIVLTQSPATLSLSPGERATLSCRASEKVDSFGNSFMH |
| | LC variable | WYQQKPGQAPRLLIYLTSNLDSGVPARFSGSGSRTDFT |
| | domain VL3/kappa | LTISSLEPEDFAVYYCQQNNEDPYTFGQGTKLEIKRTV |
| | constant domain | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |
| 152 | Humanized 52B8 | EIVLTQSPATLSLSPGERATLSCRASEKVDSFGNSFMH |
| | LC variable | WYQQKPGQAPRLLIYLTSNLDSGIPARFSGSGSGTDFT |
| | domain VL4/kappa | LTISSLEPEDFAVYYCQQNNEDPYTFGQGTKLEIKRTV |
| | constant domain | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |
| 153 | Humanized 52B8 | DIQLTQSPSSLSASVGDRVTITCRASEKVDSFGNSFMH |
| | LC variable | WYQQKPGKAPKLLIYLTSNLDSGVPSRFSGSGSGTDFT |
| | domain VL5/kappa | LTISSLQPEDFATYYCQQNNEDPYTFGQGTKLEIKRTV |
| | constant domain | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |
| 154 | Humanized 52B8 | DIQMTQSPSSLSASVGDRVTITCRASEKVDSFGNSFMH |
| | LC variable | WYQQKPGKAPKLLIYLTSNLDSGVPSRFSGSGSRTDFT |
| | domain VL6/kappa | LTISSLQPEDFATYYCQQNNEDPYTFGQGTKLEIKRTV |
| | constant domain | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |
| 155 | Humanized 52B8 | DIQLTQSPSSLSASVGDRVTITCRASEKVDSFGNSFMH |
| | LC variable | WYQQKPGKAPKLLIYLTSNLDSGVPSRFSGSGSRTDFT |
| | domain VL7/kappa | LTISSLQPEDFATYYCQQNNEDPYTFGQGTKLEIKRTV |
| | constant domain | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |
| 156 | Humanized 52B8 | DIQLTQSPSSLSASVGDRVTITCRASEKVDSFGNSFMH |
| | LC variable | WYQQKPGKAPKLLIYLTSNLDSGVPARFSGSGSRTDFT |
| | domain VL8/kappa | LTISSLQPEDFATYYCQQNNEDPYTFGQGTKLEIKRTV |
| | constant domain | AAPSVFIFPPSDEQLKSGTASVVCLLNNFYPREAKVQW |
| | | KVDNALQSGNSQESVTEQDSKDSTYSLSSTLTLSKADY |
| | | EKHKVYACEVTHQGLSSPVTKSFNRGEC |
| 157 | Humanized 52B8 | DIVLTQSPDSLAVSLGERATINCRASEKVDSFGNAFMH |
| | LC variable | WYQQKPGQPPKLLIYLTSNLDSGVPDRFSGSGSGTDFT |
| | domain VL2 | LTISSLQAEDVAVYYCQQNNEDPYTFGQGTKLEIKRTV |
| | (S35A)/kappa | AAPSVFIFPPSDEQLKSGTASVVCLLNNFYPREAKVQW |
| | constant domain | KVDNALQSGNSQESVTEQDSKDSTYSLSSTLTLSKADY |
| | | EKHKVYACEVTHQGLSSPVTKSFNRGEC |
| 158 | Humanized 52B8 | DIVLTQSPDSLAVSLGERATINCRASEKVDSFGNNFMH |
| | LC variable | WYQQKPGQPPKLLIYLTSNLDSGVPDRFSGSGSGTDFT |
| | domain VL2 | LTISSLQAEDVAVYYCQQNNEDPYTFGQGTKLEIKRTV |
| | (S35N)/kappa | AAPSVFIFPPSDEQLKSGTASVVCLLNNFYPREAKVQW |
| | constant domain | KVDNALQSGNSQESVTEQDSKDSTYSLSSTLTLSKADY |
| | | EKHKVYACEVTHQGLSSPVTKSFNRGEC |
| 159 | Humanized 52B8 | DIVLTQSPDSLAVSLGERATINCRASEKVDSFGQSFMH |
| | LC variable | WYQQKPGQPPKLLIYLTSNLDSGVPDRFSGSGSGTDFT |
| | domain VL2 | LTISSLQAEDVAVYYCQQNNEDPYTFGQGTKLEIKRTV |
| | (N34Q)/kappa | AAPSVFIFPPSDEQLKSGTASVVCLLNNFYPREAKVQW |
| | constant domain | KVDNALQSGNSQESVTEQDSKDSTYSLSSTLTLSKADY |
| | | EKHKVYACEVTHQGLSSPVTKSENRGEC |
| 160 | Humanized 52B8 | DIVLTQSPDSLAVSLGERATINCRASEKVDSFGDSFMH |
| | LC variable | WYQQKPGQPPKLLIYLTSNLDSGVPDRFSGSGSGTDFT |
| | domain VL2 | LTISSLQAEDVAVYYCQQNNEDPYTFGQGTKLEIKRTV |
| | | AAPSVFIFPPSDEQLKSGTASVVCLLNNFYPREAKVQW |
| | (N34D)/kappa | KVDNALQSGNSQESVTEQDSKDSTYSLSSTLTLSKADY |
| | constant domain | EKHKVYACEVTHQGLSSPVTKSFNRGEC |
| 161 | Humanized 52B8 | DIQLTQSPSSLSASVGDRVTITCRASEKVDSFGNAFMH |
| | LC variable | WYQQKPGKAPKLLIYLTSNLDSGVPSRFSGSGSGTDFT |
| | domain VL5 | LTISSLQPEDFATYYCQQNNEDPYTFGQGTKLEIKRTV |
| | (S35A)/kappa | AAPSVFIFPPSDEQLKSGTASVVCLLNNFYPREAKVQW |
| | constant domain | KVDNALQSGNSQESVTEQDSKDSTYSLSSTLTLSKADY |
| | | EKHKVYACEVTHQGLSSPVTKSFNRGEC |
| 162 | Humanized 52B8 | DIQLTQSPSSLSASVGDRVTITCRASEKVDSFGNNFMH |
| | LC variable | WYQQKPGKAPKLLIYLTSNLDSGVPSRFSGSGSGTDFT |
| | domain VL5 | LTISSLQPEDFATYYCQQNNEDPYTFGQGTKLEIKRTV |
| | (S35N)/kappa | AAPSVFIFPPSDEQLKSGTASVVCLLNNFYPREAKVQW |
| | constant domain | KVDNALQSGNSQESVTEQDSKDSTYSLSSTLTLSKADY |
| | | EKHKVYACEVTHQGLSSPVTKSFNRGEC |
| 163 | Humanized 52B8 | DIQLTQSPSSLSASVGDRVTITCRASEKVDSFGQSFMH |
| | LC variable | WYQQKPGKAPKLLIYLTSNLDSGVPSRFSGSGSGTDFT |
| | domain VL5 | LTISSLQPEDFATYYCQQNNEDPYTFGQGTKLEIKRTV |
| | (N34Q)/kappa | AAPSVFIFPPSDEQLKSGTASVVCLLNNFYPREAKVQW |
| | constant domain | KVDNALQSGNSQESVTEQDSKDSTYSLSSTLTLSKADY |
| | | EKHKVYACEVTHQGLSSPVTKSFNRGEC |
| 164 | Humanized 52B8 | DIQLTQSPSSLSASVGDRVTITCRASEKVDSFGDSFMH |
| | LC variable | WYQQKPGKAPKLLIYLTSNLDSGVPSRFSGSGSGTDFT |
| | domain VL5 | LTISSLQPEDFATYYCQQNNEDPYTFGQGTKLEIKRTV |
| | (N34D)/kappa | AAPSVFIFPPSDEQLKSGTASVVCLLNNFYPREAKVQW |
| | constant domain | KVDNALQSGNSQESVTEQDSKDSTYSLSSTLTLSKADY |
| | | EKHKVYACEVTHQGLSSPVTKSFNRGEC |
| 165 | Humanized 52B8 | EVQLVESGGGLVQPGGSLRLSCAASGFTFSNYGMSWV |
| | HC variable | RQAPGKGLEWVATISGGGDYTNYPDSMRGRFTISRDN |
| | domain VH1/ | AKNSLYLQMNSLRAEDTAVYYCGRRLWFRSLYYAMD |
| | Human IgG1 HC | YWGQGTLVTVSSASTKGPSVFPLAPSSKSTSGGTAALG |
| | (L234A L235A | CLVKDYFPEPVTVSWNSGALTSGVHTFPAVLQSSGLY |
| | D265S) constant | SLSSVVTVPSSSLGTQTYICNVNHKPSNTKVDKKVEPK |
| | domain | SCDKTHTCPPCPAPEAAGGPSVFLFPPKPKDTLMISRTP |
| | | EVTCVVVSVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFFL |
| | | YSKLTVDKSRWQQGNVFSCSVMHEALHNHYTQKSLS |
| | | LSPGK |
| 166 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLWFRSLYYAMD |
| | (M64V)/Human | YWGQGTLVTVSSASTKGPSVFPLAPSSKSTSGGTAALG |
| | IgG1 HC | CLVKDYFPEPVTVSWNSGALTSGVHTFPAVLQSSGLY |
| | | SLSSVVTVPSSSLGTQTYICNVNHKPSNTKVDKKVEPK |
| | | SCDKTHTCPPCPAPEAAGGPSVFLFPPKPKDTLMISRT |
| | (L234A, L235A, | PEVTCVVVSVSHEDPEVKFNWYVDGVEVHNAKTKPRE |
| | D265S) constant | EQYNSTYRVVSVLTVLHQDWLNGKEYKCKVSNKALPA |
| | domain | PIEKTISKAKGQPREPQVYTLPPSRDELTKNQVSLTC |
| | | LVKGFYPSDIAVEWESNGQPENNYKTTPPVLDSDGSF |
| | | FLYSKLTVDKSRWQQGNVFSCSVMHEALHNHYTQKSLS |
| | | LSPGK |
| 167 | Humanized 52B8 | EVQLVESGGGLVQPGGSLRLSCAASGFTFSNYGMSWV |
| | HC variable | RQAPGKGLEWVATISGGGDYTNYPDSLRGRFTISRDN |
| | domain VH1 | AKNSLYLQMNSLRAEDTAVYYCGRRLWFRSLYYAMDY |
| | (M64L)/Human | WGQGTLVTVSSASTKGPSVFPLAPSSKSTSGGTAALG |
| | IgG1 HC | CLVKDYFPEPVTVSWNSGALTSGVHTFPAVLQSSGLY |
| | (L234A, L235A, | SLSSVVTVPSSSLGTQTYICNVNHKPSNTKVDKKVEPK |
| | D265S) constant | SCDKTHTCPPCPAPEAAGGPSVFLFPPKPKDTLMISR |
| | domain | TPEVTCVVVSVSHEDPEVKFNWYVDGVEVHNAKTKPR |
| | | EEQYNSTYRVVSVLTVLHQDWLNGKEYKCKVSNKALP |
| | | APIEKTISKAKGQPREPQVYTLPPSRDELTKNQVSLT |
| | | CLVKGFYPSDIAVEWESNGQPENNYKTTPPVLDSDGS |
| | | FFLYSKLTVDKSRWQQGNVFSCSVMHEALHNHYTQKS |
| | | LSLSPGK |
| 168 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLFFRSLYYAMD |
| | (M64V, W101F)/ | YWGQGTLVTVSSASTKGPSVFPLAPSSKSTSGGTAALG |
| | Human IgG1 HC | CLVKDYFPEPVTVSWNSGALTSGVHTFPAVLQSSGLY |
| | (L234A, L235A, | SLSSVVTVPSSSLGTQTYICNVNHKPSNTKVDKKVEPK |
| | D265S) constant | SCDKTHTCPPCPAPEAAGGPSVFLFPPKPKDTLMISRTP |
| | domain | EVTCVVVSVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFFL |
| | | YSKLTVDKSRWQQGNVFSCSVMHEALHNHYTQKSLS |
| | | LSPGK |
| 169 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLYFRSLYYAMD |
| | (M64V, W101Y)/ | YWGQGTLVTVSSASTKGPSVFPLAPSSKSTSGGTAALG |
| | Human IgG1 HC | CLVKDYFPEPVTVSWNSGALTSGVHTFPAVLQSSGLY |
| | (L234A, L235A, | SLSSVVTVPSSSLGTQTYICNVNHKPSNTKVDKKVEPK |
| | D265S) constant | SCDKTHTCPPCPAPEAAGGPSVFLFPPKPKDTLMISRTP |
| | domain | EVTCVVVSVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFFL |
| | | YSKLTVDKSRWQQGNVFSCSVMHEALHNHYTQKSLS |
| | | LSPGK |
| 170 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLQFRSLYYAMD |
| | (M64V, W101Q)/ | YWGQGTLVTVSSASTKGPSVFPLAPSSKSTSGGTAALG |
| | Human IgG1 HC | CLVKDYFPEPVTVSWNSGALTSGVHTFPAVLQSSGLY |
| | (L234A, L235A, | SLSSVVTVPSSSLGTQTYICNVNHKPSNTKVDKKVEPK |
| | D265S) constant | SCDKTHTCPPCPAPEAAGGPSVFLFPPKPKDTLMISRTP |
| | domain | EVTCVVVSVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFFL |
| | | YSKLTVDKSRWQQGNVFSCSVMHEALHNHYTQKSLS |
| | | LSPGK |
| 171 | Humanized 52B8 | EVQLVESGGGLVQPGGSLRLSCAASGFTFSNYGMSWV |
| | HC variable | RQAPGKGLEWVATISGGGDYTNYPDSMRGRFTISRDN |
| | domain VH2/ | AKNSLYLQMNSLKAEDTAVYYCGRRLWFRSLYYAM |
| | Human IgG1 HC | DYWGQGTLVTVSSASTKGPSVFPLAPSSKSTSGGTAAL |
| | (L234A, L235A, | GCLVKDYFPEPVTVSWNSGALTSGVHTFPAVLQSSGL |
| | D265S) constant | YSLSSVVTVPSSSLGTQTYICNVNHKPSNTKVDKKVEP |
| | domain | KSCDKTHTCPPCPAPEAAGGPSVFLFPPKPKDTLMISRT |
| | | PEVTCVVVSVSHEDPEVKFNWYVDGVEVHNAKTKPR |
| | | EEQYNSTYRVVSVLTVLHQDWLNGKEYKCKVSNKAL |
| | | PAPIEKTISKAKGQPREPQVYTLPPSRDELTKNQVSLTC |
| | | LVKGFYPSDIAVEWESNGQPENNYKTTPPVLDSDGSFF |
| | | LYSKLTVDKSRWQQGNVFSCSVMHEALHNHYTQKSL |
| | | SLSPGK |
| 172 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH2 | AKNSLYLQMNSLKAEDTAVYYCGRRLWFRSLYYAM |
| | (M64V)/Human | DYWGQGTLVTVSSASTKGPSVFPLAPSSKSTSGGTAAL |
| | IgG1 HC | GCLVKDYFPEPVTVSWNSGALTSGVHTFPAVLQSSGL |
| | (L234A, L235A, | YSLSSVVTVPSSSLGTQTYICNVNHKPSNTKVDKKVEP |
| | D265S) constant | KSCDKTHTCPPCPAPEAAGGPSVFLFPPKPKDTLMISRT |
| | domain | PEVTCVVVSVSHEDPEVKFNWYVDGVEVHNAKTKPR |
| | | EEQYNSTYRVVSVLTVLHQDWLNGKEYKCKVSNKAL |
| | | PAPIEKTISKAKGQPREPQVYTLPPSRDELTKNQVSLTC |
| | | LVKGFYPSDIAVEWESNGQPENNYKTTPPVLDSDGSFF |
| | | LYSKLTVDKSRWQQGNVFSCSVMHEALHNHYTQKSL |
| | | SLSPGK |
| 173 | Humanized 52B8 | EVQLVESGGGLVQPGGSLRLSCAASGFTFSNYGMSWV |
| | HC variable | RQAPGKGLEWVATISGGGDYTNYPDSLRGRFTISRDN |
| | domain VH2 | AKNSLYLQMNSLKAEDTAVYYCGRRLWFRSLYYAM |
| | (M64L)/Human | DYWGQGTLVTVSSASTKGPSVFPLAPSSKSTSGGTAAL |
| | IgG1 HC | GCLVKDYFPEPVTVSWNSGALTSGVHTFPAVLQSSGL |
| | (L234A, L235A, | YSLSSVVTVPSSSLGTQTYICNVNHKPSNTKVDKKVEP |
| | D265S) constant | KSCDKTHTCPPCPAPEAAGGPSVFLFPPKPKDTLMISRT |
| | domain | PEVTCVVVSVSHEDPEVKFNWYVDGVEVHNAKTKPR |
| | | EEQYNSTYRVVSVLTVLHQDWLNGKEYKCKVSNKAL |
| | | PAPIEKTISKAKGQPREPQVYTLPPSRDELTKNQVSLTC |
| | | LVKGFYPSDIAVEWESNGQPENNYKTTPPVLDSDGSFF |
| | | LYSKLTVDKSRWQQGNVFSCSVMHEALHNHYTQKSL |
| | | SLSPGK |
| 174 | Humanized 52B8 | EVQLVESGGGLVQPGGSLRLSCAASGFTFSNYGMSWV |
| | HC variable | RQAPGKGLEWVATISGGGDYTNYPDSMRGRFTISRDN |
| | domain | AKNSLYLQMNSLRAEDTAVYYCGRRLWFRSLYYAMD |
| | VH1/Human IgG4 | YWGQGTLVTVSSASTKGPSVFPLAPCSRSTSESTAALG |
| | (S228P) (K-) | CLVKDYFPEPVTVSWNSGALTSGVHTFPAVLQSSGLY |
| | constant domain | SLSSVVTVPSSSLGTKTYTCNVDHKPSNTKVDKRVESK |
| | | YGPPCPPCPAPEFLGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIE |
| | | KTISKAKGQPREPQVYTLPPSQEEMTKNQVSLTCLVKG |
| | | FYPSDIAVEWESNGQPENNYKTTPPVLDSDGSFFLYSR |
| | | LTVDKSRWQEGNVFSCSVMHEALHNHYTQKSLSLSLG |
| 175 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLWFRSLYYAMD |
| | (M64V)/Human | YWGQGTLVTVSSASTKGPSVFPLAPCSRSTSESTAALG |
| | IgG4 (S228P) (K-) | CLVKDYFPEPVTVSWNSGALTSGVHTFPAVLQSSGLY |
| | constant domain | SLSSVVTVPSSSLGTKTYTCNVDHKPSNTKVDKRVESK |
| | | YGPPCPPCPAPEFLGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIE |
| | | KTISKAKGQPREPQVYTLPPSQEEMTKNQVSLTCLVKG |
| | | FYPSDIAVEWESNGQPENNYKTTPPVLDSDGSFFLYSR |
| | | LTVDKSRWQEGNVFSCSVMHEALHNHYTQKSLSLSLG |
| 176 | Humanized 52B8 | EVQLVESGGGLVQPGGSLRLSCAASGFTFSNYGMSWV |
| | HC variable | RQAPGKGLEWVATISGGGDYTNYPDSLRGRFTISRDN |
| | domain VH1 | AKNSLYLQMNSLRAEDTAVYYCGRRLWFRSLYYAMD |
| | (M64L)/Human | YWGQGTLVTVSSASTKGPSVFPLAPCSRSTSESTAALG |
| | IgG4 (S228P) (K-) | CLVKDYFPEPVTVSWNSGALTSGVHTFPAVLQSSGLY |
| | constant domain | SLSSVVTVPSSSLGTKTYTCNVDHKPSNTKVDKRVESK |
| | | YGPPCPPCPAPEFLGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIE |
| | | KTISKAKGQPREPQVYTLPPSQEEMTKNQVSLTCLVKG |
| | | FYPSDIAVEWESNGQPENNYKTTPPVLDSDGSFFLYSR |
| | | LTVDKSRWQEGNVFSCSVMHEALHNHYTQKSLSLSLG |
| 177 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLFFRSLYYAMD |
| | (M64V), | YWGQGTLVTVSSASTKGPSVFPLAPCSRSTSESTAALG |
| | W101F/Human | CLVKDYFPEPVTVSWNSGALTSGVHTFPAVLQSSGLY |
| | IgG4 (S228P) (K-) | SLSSVVTVPSSSLGTKTYTCNVDHKPSNTKVDKRVESK |
| | constant domain | YGPPCPPCPAPEFLGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIE |
| | | KTISKAKGQPREPQVYTLPPSQEEMTKNQVSLTCLVKG |
| | | FYPSDIAVEWESNGQPENNYKTTPPVLDSDGSFFLYSR |
| | | LTVDKSRWQEGNVFSCSVMHEALHNHYTQKSLSLSLG |
| 178 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLYFRSLYYAMD |
| | (M64V, | YWGQGTLVTVSSASTKGPSVFPLAPCSRSTSESTAALG |
| | W101Y)/Human | CLVKDYFPEPVTVSWNSGALTSGVHTFPAVLQSSGLY |
| | IgG4 (S228P) (K-) | SLSSVVTVPSSSLGTKTYTCNVDHKPSNTKVDKRVESK |
| | constant domain | YGPPCPPCPAPEFLGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIE |
| | | KTISKAKGQPREPQVYTLPPSQEEMTKNQVSLTCLVKG |
| | | FYPSDIAVEWESNGQPENNYKTTPPVLDSDGSFFLYSR |
| | | LTVDKSRWQEGNVFSCSVMHEALHNHYTQKSLSLSLG |
| 179 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLQFRSLYYAMD |
| | (M64V, | YWGQGTLVTVSSASTKGPSVFPLAPCSRSTSESTAALG |
| | 101Q)/Human | CLVKDYFPEPVTVSWNSGALTSGVHTFPAVLQSSGLY |
| | IgG4 (S228P) (K-) | SLSSVVTVPSSSLGTKTYTCNVDHKPSNTKVDKRVESK |
| | constant domain | YGPPCPPCPAPEFLGGPSVFLFPPKPKDTLMISRTPEVT |
| | | CVVVDVSQEDPEVQFNWYVDGVEVHNAKTKPREEQF |
| | | NSTYRVVSVLTVLHQDWLNGKEYKCKVSNKGLPSSIE |
| | | KTISKAKGQPREPQVYTLPPSQEEMTKNQVSLTCLVKG |
| | | FYPSDIAVEWESNGQPENNYKTTPPVLDSDGSFFLYSR |
| | | LTVDKSRWQEGNVFSCSVMHEALHNHYTQKSLSLSLG |
| 180 | Humanized 52B8 | EVQLVESGGGLVQPGGSLRLSCAASGFTFSNYGMSWV |
| | HC variable | RQAPGKGLEWVATISGGGDYTNYPDSMRGRFTISRDN |
| | domain | AKNSLYLQMNSLKAEDTAVYYCGRRLWFRSLYYAM |
| | VH2/Human IgG4 | DYWGQGTLVTVSSASTKGPSVFPLAPCSRSTSESTAAL |
| | (S228P) (K-) | GCLVKDYFPEPVTVSWNSGALTSGVHTFPAVLQSSGL |
| | constant domain | YSLSSVVTVPSSSLGTKTYTCNVDHKPSNTKVDKRVES |
| | | KYGPPCPPCPAPEFLGGPSVFLFPPKPKDTLMISRTPEV |
| | | TCVVVDVSQEDPEVQFNWYVDGVEVHNAKTKPREEQ |
| | | FNSTYRVVSVLTVLHQDWLNGKEYKCKVSNKGLPSSI |
| | | EKTISKAKGQPREPQVYTLPPSQEEMTKNQVSLTCLVK |
| | | GFYPSDIAVEWESNGQPENNYKTTPPVLDSDGSFFLYS |
| | | RLTVDKSRWQEGNVFSCSVMHEALHNHYTQKSLSLSL |
| | | G |
| 181 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH2 | AKNSLYLQMNSLKAEDTAVYYCGRRLWFRSLYYAM |
| | (M64V)/Human | DYWGQGTLVTVSSASTKGPSVFPLAPCSRSTSESTAAL |
| | IgG4 (S228P) (K-) | GCLVKDYFPEPVTVSWNSGALTSGVHTFPAVLQSSGL |
| | constant domain | YSLSSVVTVPSSSLGTKTYTCNVDHKPSNTKVDKRVES |
| | | KYGPPCPPCPAPEFLGGPSVFLFPPKPKDTLMISRTPEV |
| | | TCVVVDVSQEDPEVQFNWYVDGVEVHNAKTKPREEQ |
| | | FNSTYRVVSVLTVLHQDWLNGKEYKCKVSNKGLPSSI |
| | | EKTISKAKGQPREPQVYTLPPSQEEMTKNQVSLTCLVK |
| | | GFYPSDIAVEWESNGQPENNYKTTPPVLDSDGSFFLYS |
| | | RLTVDKSRWQEGNVFSCSVMHEALHNHYTQKSLSLSL |
| | | G |
| 182 | Humanized 52B8 | EVQLVESGGGLVQPGGSLRLSCAASGFTFSNYGMSWV |
| | HC variable | RQAPGKGLEWVATISGGGDYTNYPDSLRGRFTISRDN |
| | domain VH2 | AKNSLYLQMNSLKAEDTAVYYCGRRLWFRSLYYAM |
| | (M64L)/Human | DYWGQGTLVTVSSASTKGPSVFPLAPCSRSTSESTAAL |
| | IgG4 (S228P) (K-) | GCLVKDYFPEPVTVSWNSGALTSGVHTFPAVLQSSGL |
| | constant domain | YSLSSVVTVPSSSLGTKTYTCNVDHKPSNTKVDKRVES |
| | | KYGPPCPPCPAPEFLGGPSVFLFPPKPKDTLMISRTPEV |
| | | TCVVVDVSQEDPEVQFNWYVDGVEVHNAKTKPREEQ |
| | | FNSTYRVVSVLTVLHQDWLNGKEYKCKVSNKGLPSSI |
| | | EKTISKAKGQPREPQVYTLPPSQEEMTKNQVSLTCLVK |
| | | GFYPSDIAVEWESNGQPENNYKTTPPVLDSDGSFFLYS |
| | | RLTVDKSRWQEGNVFSCSVMHEALHNHYTQKSLSLSL |
| | | G |
| 183 | Humanized 52B8 | EVQLVESGGGLVQPGGSLRLSCAASGFTFSNYGMSWV |
| | HC variable | RQAPGKGLEWVATISGGGDYTNYPDSMRGRFTISRDN |
| | domain VH1/ | AKNSLYLQMNSLRAEDTAVYYCGRRLWFRSLYYAMD |
| | Human IgG1 HC | YWGQGTLVTVSSASTKGPSVFPLAPSSKSTSGGTAALG |
| | (L234A, L235A, | CLVKDYFPEPVTVSWNSGALTSGVHTFPAVLQSSGLY |
| | D265S) (K-) | SLSSVVTVPSSSLGTQTYICNVNHKPSNTKVDKKVEPK |
| | constant domain | SCDKTHTCPPCPAPEAAGGPSVFLFPPKPKDTLMISRTP |
| | | EVTCVVVSVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFFL |
| | | YSKLTVDKSRWQQGNVFSCSVMHEALHNHYTQKSLS |
| | | LSPG |
| 184 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLWFRSLYYAMD |
| | (M64V)/Human | YWGQGTLVTVSSASTKGPSVFPLAPSSKSTSGGTAALG |
| | IgG1 HC | CLVKDYFPEPVTVSWNSGALTSGVHTFPAVLQSSGLY |
| | (L234A, L235A, | SLSSVVTVPSSSLGTQTYICNVNHKPSNTKVDKKVEPK |
| | D265S) (K-) | SCDKTHTCPPCPAPEAAGGPSVFLFPPKPKDTLMISRTP |
| | constant domain | EVTCVVVSVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFFL |
| | | YSKLTVDKSRWQQGNVFSCSVMHEALHNHYTQKSLS |
| | | LSPG |
| 185 | Humanized 52B8 | EVQLVESGGGLVQPGGSLRLSCAASGFTFSNYGMSWV |
| | HC variable | RQAPGKGLEWVATISGGGDYTNYPDSLRGRFTISRDN |
| | domain VH1 | AKNSLYLQMNSLRAEDTAVYYCGRRLWFRSLYYAMD |
| | (M64L)/Human | YWGQGTLVTVSSASTKGPSVFPLAPSSKSTSGGTAALG |
| | IgG1 HC | CLVKDYFPEPVTVSWNSGALTSGVHTFPAVLQSSGLY |
| | (L234A, L235A, | SLSSVVTVPSSSLGTQTYICNVNHKPSNTKVDKKVEPK |
| | D265S) (K-) | SCDKTHTCPPCPAPEAAGGPSVFLFPPKPKDTLMISRTP |
| | constant domain | EVTCVVVSVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFFL |
| | | YSKLTVDKSRWQQGNVFSCSVMHEALHNHYTQKSLS |
| | | LSPG |
| 186 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLFFRSLYYAMD |
| | (M64V, W101F)/ | YWGQGTLVTVSSASTKGPSVFPLAPSSKSTSGGTAALG |
| | Human IgG1 HC | CLVKDYFPEPVTVSWNSGALTSGVHTFPAVLQSSGLY |
| | (L234A, L235A, | SLSSVVTVPSSSLGTQTYICNVNHKPSNTKVDKKVEPK |
| | D265S) (K-) | SCDKTHTCPPCPAPEAAGGPSVFLFPPKPKDTLMISRTP |
| | constant domain | EVTCVVVSVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFFL |
| | | YSKLTVDKSRWQQGNVFSCSVMHEALHNHYTQKSLS |
| | | LSPG |
| 187 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLYFRSLYYAMD |
| | (M64V, W101Y)/ | YWGQGTLVTVSSASTKGPSVFPLAPSSKSTSGGTAALG |
| | Human IgG1 HC | CLVKDYFPEPVTVSWNSGALTSGVHTFPAVLQSSGLY |
| | (L234A, L235A, | SLSSVVTVPSSSLGTQTYICNVNHKPSNTKVDKKVEPK |
| | D265S) (K-) | SCDKTHTCPPCPAPEAAGGPSVFLFPPKPKDTLMISRTP |
| | constant domain | EVTCVVVSVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFFL |
| | | YSKLTVDKSRWQQGNVFSCSVMHEALHNHYTQKSLS |
| | | LSPG |
| 188 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLQFRSLYYAMD |
| | (M64V, W101Q)/ | YWGQGTLVTVSSASTKGPSVFPLAPSSKSTSGGTAALG |
| | Human IgG1 HC | CLVKDYFPEPVTVSWNSGALTSGVHTFPAVLQSSGLY |
| | (L234A, L235A, | SLSSVVTVPSSSLGTQTYICNVNHKPSNTKVDKKVEPK |
| | D265S) (K-) | SCDKTHTCPPCPAPEAAGGPSVFLFPPKPKDTLMISRTP |
| | constant domain | EVTCVVVSVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFFL |
| | | YSKLTVDKSRWQQGNVFSCSVMHEALHNHYTQKSLS |
| | | LSPG |
| 189 | Humanized 52B8 | EVQLVESGGGLVQPGGSLRLSCAASGFTFSNYGMSWV |
| | HC variable | RQAPGKGLEWVATISGGGDYTNYPDSMRGRFTISRDN |
| | domain VH2/ | AKNSLYLQMNSLKAEDTAVYYCGRRLWFRSLYYAM |
| | Human IgG1 HC | DYWGQGTLVTVSSASTKGPSVFPLAPSSKSTSGGTAAL |
| | (L234A, L235A, | GCLVKDYFPEPVTVSWNSGALTSGVHTFPAVLQSSGL |
| | D265S) (K-) | YSLSSVVTVPSSSLGTQTYICNVNHKPSNTKVDKKVEP |
| | constant domain | KSCDKTHTCPPCPAPEAAGGPSVFLFPPKPKDTLMISRT |
| | | PEVTCVVVSVSHEDPEVKFNWYVDGVEVHNAKTKPR |
| | | EEQYNSTYRVVSVLTVLHQDWLNGKEYKCKVSNKAL |
| | | PAPIEKTISKAKGQPREPQVYTLPPSRDELTKNQVSLTC |
| | | LVKGFYPSDIAVEWESNGQPENNYKTTPPVLDSDGSFF |
| | | LYSKLTVDKSRWQQGNVFSCSVMHEALHNHYTQKSL |
| | | SLSPG |
| 190 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH2 | AKNSLYLQMNSLKAEDTAVYYCGRRLWFRSLYYAM |
| | M64V/Human | DYWGQGTLVTVSSASTKGPSVFPLAPSSKSTSGGTAAL |
| | IgG1 HC | GCLVKDYFPEPVTVSWNSGALTSGVHTFPAVLQSSGL |
| | (L234A, L235A, | YSLSSVVTVPSSSLGTQTYICNVNHKPSNTKVDKKVEP |
| | D265S) (K-) | KSCDKTHTCPPCPAPEAAGGPSVFLFPPKPKDTLMISRT |
| | constant domain | PEVTCVVVSVSHEDPEVKFNWYVDGVEVHNAKTKPR |
| | | EEQYNSTYRVVSVLTVLHQDWLNGKEYKCKVSNKAL |
| | | PAPIEKTISKAKGQPREPQVYTLPPSRDELTKNQVSLTC |
| | | LVKGFYPSDIAVEWESNGQPENNYKTTPPVLDSDGSFF |
| | | LYSKLTVDKSRWQQGNVFSCSVMHEALHNHYTQKSL |
| | | SLSPG |
| 191 | Humanized 52B8 | EVQLVESGGGLVQPGGSLRLSCAASGFTFSNYGMSWV |
| | HC variable | RQAPGKGLEWVATISGGGDYTNYPDSLRGRFTISRDN |
| | domain VH2 | AKNSLYLQMNSLKAEDTAVYYCGRRLWFRSLYYAM |
| | M64L/Human | DYWGQGTLVTVSSASTKGPSVFPLAPSSKSTSGGTAAL |
| | IgG1 HC | GCLVKDYFPEPVTVSWNSGALTSGVHTFPAVLQSSGL |
| | (L234A, L235A, | YSLSSVVTVPSSSLGTQTYICNVNHKPSNTKVDKKVEP |
| | D265S) (K-) | KSCDKTHTCPPCPAPEAAGGPSVFLFPPKPKDTLMISRT |
| | constant domain | PEVTCVVVSVSHEDPEVKFNWYVDGVEVHNAKTKPR |
| | | EEQYNSTYRVVSVLTVLHQDWLNGKEYKCKVSNKAL |
| | | PAPIEKTISKAKGQPREPQVYTLPPSRDELTKNQVSLTC |
| | | LVKGFYPSDIAVEWESNGQPENNYKTTPPVLDSDGSFF |
| | | LYSKLTVDKSRWQQGNVFSCSVMHEALHNHYTQKSL |
| | | SLSPG |
| 192 | Chimeric Anti- | QVQLKESGPGLVQASETLSLTCTVSGFSLTSYSINWVR |
| | ILT3 rat 40A6 | QSSGKGPEWMGRFWYDEGIAYNLTLESRLSISGDTSK |
| | parental HC | NQVFLKMNSLRTGDTGTYYCTRDRDTVGITGWFAYW |
| | variable | GQGTLVTVSSASTKGPSVFPLAPSSKSTSGGTAALGCL |
| | domain/human | VKDYFPEPVTVSWNSGALTSGVHTFPAVLQSSGLYSLS |
| | IgG4 (S228P) | SVVTVPSSSLGTQTYICNVNHKPSNTKVDKKVEPKSCD |
| | constant domain | KTHTCPPCPAPEAAGGPSVFLFPPKPKDTLMISRTPEVT |
| | | CVVVSVSHEDPEVKFNWYVDGVEVHNAKTKPREEQY |
| | | NSTYRVVSVLTVLHQDWLNGKEYKCKVSNKALPAPIE |
| | | KTISKAKGQPREPQVYTLPPSRDELTKNQVSLTCLVKG |
| | | FYPSDIAVEWESNGQPENNYKTTPPVLDSDGSFFLYSK |
| | | LTVDKSRWQQGNVFSCSVMHEALHNHYTQKSLSLSP |
| | | GK |
| 193 | Chimeric Anti- | ETVMTQSPTSLSASIGERVTLNCKASQSVGVNVDWYQ |
| | ILT3 rat 40A6 | QTPGQSPKLLIYGSANRHTGVPDRFTGSGFGSDFTLTIS |
| | parental LC | DVEPEDLGVYYCLQYGSVPYTFGAGTKLELKRTVAAP |
| | variable | SVFIFPPSDEQLKSGTASVVCLLNNFYPREAKVQWKVD |
| | domain/human | NALQSGNSQESVTEQDSKDSTYSLSSTLTLSKADYEKH |
| | kappa | KVYACEVTHQGLSSPVTKSFNRGEC |
| 194 | Chimeric Anti- | QVQLKESGPGLVQASETLSLTCTVSGFSLTNYCVNWV |
| | ILT3 rat 16B1 | RQPSGKGPEWLGRFWFDEGKAYNLTLESRLSISGDTSK |
| | parental HC | NQVFLRMNSLRADDTGTYYCTRDRDTVGITGWFAYW |
| | variable | GQGTLVTVSSASTKGPSVFPLAPSSKSTSGGTAALGCL |
| | domain/human | VKDYFPEPVTVSWNSGALTSGVHTFPAVLQSSGLYSLS |
| | IgG4 (S228P) | SVVTVPSSSLGTQTYICNVNHKPSNTKVDKKVEPKSCD |
| | constant domain | KTHTCPPCPAPEAAGGPSVFLFPPKPKDTLMISRTPEVT |
| | | CVVVSVSHEDPEVKFNWYVDGVEVHNAKTKPREEQY |
| | | NSTYRVVSVLTVLHQDWLNGKEYKCKVSNKALPAPIE |
| | | KTISKAKGQPREPQVYTLPPSRDELTKNQVSLTCLVKG |
| | | FYPSDIAVEWESNGQPENNYKTTPPVLDSDGSFFLYSK |
| | | LTVDKSRWQQGNVFSCSVMHEALHNHYTQKSLSLSP |
| | | GK |
| 195 | Chimeric Anti- | ETVMTQSPTSLSASIGERVTLNCKASQSVGINVDWYQ |
| | ILT3 rat 16B1 | QTPGQSPKLLIYGSANRHTGVPDRFTGSGFGSDFTLTIS |
| | parental LC | NVEPEDLGVYYCLQYGSVPYTFGPGTKLELKRTVAAP |
| | variable | SVFIFPPSDEQLKSGTASVVCLLNNFYPREAKVQWKVD |
| | domain/human | NALQSGNSQESVTEQDSKDSTYSLSSTLTLSKADYEKH |
| | kappa | KVYACEVTHQGLSSPVTKSFNRGEC |
| 196 | Chimeric Anti- | QVQLQQSGAELMKPGASVKISCKATGYTFRTYWIEWV |
| | ILT3 mouse 11D1 | KQRPGHGLEWIGEILPGNGNTHFNENFKDKATFTADTS |
| | parental HC | SNAAYMQLSSLTSEDSAVYYCVRRLGRGPFDFWGQG |
| | variable | TTLTVSSASTKGPSVFPLAPSSKSTSGGTAALGCLVKD |
| | domain/human | YFPEPVTVSWNSGALTSGVHTFPAVLQSSGLYSLSSVV |
| | IgG4 (S228P) | TVPSSSLGTQTYICNVNHKPSNTKVDKKVEPKSCDKTH |
| | constant domain | TCPPCPAPEAAGGPSVFLFPPKPKDTLMISRTPEVTCVV |
| | | VSVSHEDPEVKFNWYVDGVEVHNAKTKPREEQYNST |
| | | YRVVSVLTVLHQDWLNGKEYKCKVSNKALPAPIEKTI |
| | | SKAKGQPREPQVYTLPPSRDELTKNQVSLTCLVKGFYP |
| | | SDIAVEWESNGQPENNYKTTPPVLDSDGSFFLYSKLTV |
| | | DKSRWQQGNVFSCSVMHEALHNHYTQKSLSLSPGK |
| 197 | Chimeric Anti- | DIQMTQSPSSLSVSLGGKVTITCKASQDINEYIGWYQR |
| | ILT3 mouse 11D1 | KPGKGPRLLIHYTSTLQSGIPSRFSGSGSGRDYSLSISNL |
| | parental LC | EPEDIATYYCLQYANPLPTFGGGTKLEIKRTVAAPSVFI |
| | variable | FPPSDEQLKSGTASVVCLLNNFYPREAKVQWKVDNAL |
| | domain/human | QSGNSQESVTEQDSKDSTYSLSSTLTLSKADYEKHKVY |
| | kappa | ACEVTHQGLSSPVTKSFNRGEC |
| 198 | Chimeric Anti- | EVQLVESGGGLVQPGRSMKLSCAASGFTFSNFDMAW |
| | ILT3 rat 17H12 | VRQAPTRGLEWVSSITYDGGSTSYRDSVKGRFTISRDN |
| | parental HC | AKGTLYLQMDSLRSEDTATYYCTTVESIATISTYFDYW |
| | variable | GQGVMVTVSSASTKGPSVFPLAPSSKSTSGGTAALGCL |
| | domain/human | VKDYFPEPVTVSWNSGALTSGVHTFPAVLQSSGLYSLS |
| | IgG4 (S228P) | SVVTVPSSSLGTQTYICNVNHKPSNTKVDKKVEPKSCD |
| | constant domain | KTHTCPPCPAPEAAGGPSVFLFPPKPKDTLMISRTPEVT |
| | | CVVVSVSHEDPEVKFNWYVDGVEVHNAKTKPREEQY |
| | | NSTYRVVSVLTVLHQDWLNGKEYKCKVSNKALPAPIE |
| | | KTISKAKGQPREPQVYTLPPSRDELTKNQVSLTCLVKG |
| | | FYPSDIAVEWESNGQPENNYKTTPPVLDSDGSFFLYSK |
| | | LTVDKSRWQQGNVFSCSVMHEALHNHYTQKSLSLSP |
| | | GK |
| 199 | Chimeric Anti- | DIVLTQSPALAVSLGQRATISCRASQSVSMSRYDLIHW |
| | ILT3 rat 17H12 | YQQKPGQQPKLLIFRASDLASGIPARFSGSGSGTDFTLT |
| | parental LC | INPVQADDIATYYCQQTRKSPPTFGGGTRLELKRTVAA |
| | variable | PSVFIFPPSDEQLKSGTASVVCLLNNFYPREAKVQWKV |
| | domain/human | DNALQSGNSQESVTEQDSKDSTYSLSSTLTLSKADYEK |
| | kappa | HKVYACEVTHQGLSSPVTKSFNRGEC |
| 200 | Chimeric Anti- | QVQLKESGPGLVQASETLSLTCTVSGFSLTSYCVNWV |
| | ILT3 rat 37C8 | RQPSGKGPEWLGRFWYDEGKVYNLTLESRLSISGDTS |
| | parental HC | KNQVFLKMNRLRTDDTGTYYCTRDRDTMGITGWFAY |
| | variable | WGQGTLVTVSSASTKGPSVFPLAPSSKSTSGGTAALGC |
| | domain/human | LVKDYFPEPVTVSWNSGALTSGVHTFPAVLQSSGLYSL |
| | IgG4 (S228P) | SSVVTVPSSSLGTQTYICNVNHKPSNTKVDKKVEPKSC |
| | constant domain | DKTHTCPPCPAPEAAGGPSVFLFPPKPKDTLMISRTPEV |
| | | TCVVVSVSHEDPEVKFNWYVDGVEVHNAKTKPREEQ |
| | | YNSTYRVVSVLTVLHQDWLNGKEYKCKVSNKALPAPI |
| | | EKTISKAKGQPREPQVYTLPPSRDELTKNQVSLTCLVK |
| | | GFYPSDIAVEWESNGQPENNYKTTPPVLDSDGSFFLYS |
| | | KLTVDKSRWQQGNVFSCSVMHEALHNHYTQKSLSLS |
| | | PGK |
| 201 | Chimeric Anti- | ETVMTQSPTSLSASIGERVTLNCKASQSVGINVDWYQ |
| | ILT3 rat 37C8 | QTPGQSPKLLIYGSANRHTGVPDRFTGSGFGSGFTLTIS |
| | parental LC | NVEPEDLGVYYCLQYGSVPYTFGPGTKLELKRTVAAP |
| | variable | SVFIFPPSDEQLKSGTASVVCLLNNFYPREAKVQWKVD |
| | domain/human | NALQSGNSQESVTEQDSKDSTYSLSSTLTLSKADYEKH |
| | kappa | KVYACEVTHQGLSSPVTKSFNRGEC |
| 202 | Chimeric Anti- | QVQMQQSGTELMKPGASMKISCKATGYTFSTYWIQWI |
| | ILT3 mouse 1G12 | KQRPGHGLEWIGEILPGSGTTNYNENFKGKATFSADTS |
| | parental HC | SNTAYIHLSSLTSEDSAVFYCARRLGRGPFDYWGQGTT |
| | variable | LTVSSASTKGPSVFPLAPSSKSTSGGTAALGCLVKDYF |
| | domain/human | PEPVTVSWNSGALTSGVHTFPAVLQSSGLYSLSSVVTV |
| | IgG4 (S228P) | PSSSLGTQTYICNVNHKPSNTKVDKKVEPKSCDKTHTC |
| | constant domain | PPCPAPEAAGGPSVFLFPPKPKDTLMISRTPEVTCVVVS |
| | | VSHEDPEVKFNWYVDGVEVHNAKTKPREEQYNSTYR |
| | | VVSVLTVLHQDWLNGKEYKCKVSNKALPAPIEKTISK |
| | | AKGQPREPQVYTLPPSRDELTKNQVSLTCLVKGFYPSD |
| | | IAVEWESNGQPENNYKTTPPVLDSDGSFFLYSKLTVDK |
| | | SRWQQGNVFSCSVMHEALHNHYTQKSLSLSPGK |
| 203 | Chimeric Anti- | DIQMTQSPSSLSASLGGKVTITCEASQDINKHIDWYQHQ |
| | ILT3 mouse 1G12 | PGRGPSLLIHYASILQPGIPSRFSGSGSGRDYSFSITSL |
| | parental LC | EPEDIATYYCLQYDNLLPTFGGGTKLEIKRTVAAPSVFI |
| | variable | FPPSDEQLKSGTASVVCLLNNFYPREAKVQWKVDNALQS |
| | domain/human | GNSQESVTEQDSKDSTYSLSSTLTLSKADYEKHKVYACE |
| | kappa | VTHQGLSSPVTKSFNRGEC |
| 204 | Chimeric Anti- | QVQLKESGPGLVQASETLSLTCTVSGFSLTSYSVNWVR |
| | ILT3 rat 20E4 | QPSGKGLEWMGRFWYDGGTAYNSTLESRLSISGDTSK |
| | parental HC | NQVFLKMNSLQTDDTGTYYCTRDRDTMGITGWFAYW |
| | variable | GQGTLVTVSPASTKGPSVFPLAPSSKSTSGGTAALGCL |
| | domain/human | VKDYFPEPVTVSWNSGALTSGVHTFPAVLQSSGLYSLS |
| | IgG4 (S228P) | SVVTVPSSSLGTQTYICNVNHKPSNTKVDKKVEPKSCD |
| | constant domain | KTHTCPPCPAPEAAGGPSVFLFPPKPKDTLMISRTPEVT |
| | | CVVVSVSHEDPEVKFNWYVDGVEVHNAKTKPREEQY |
| | | NSTYRVVSVLTVLHQDWLNGKEYKCKVSNKALPAPIE |
| | | KTISKAKGQPREPQVYTLPPSRDELTKNQVSLTCLVKG |
| | | FYPSDIAVEWESNGQPENNYKTTPPVLDSDGSFFLYSK |
| | | LTVDKSRWQQGNVFSCSVMHEALHNHYTQKSLSLSP |
| | | GK |
| 205 | Chimeric Anti- | ETVMTQSPTSLSASIGERVTLNCKASQSVGVNVDWYQ |
| | ILT3 rat 20E4 | QTPGQSPKLLIYGSANRHTGVPDRFTGSGFGSDFTLTI |
| | parental LC | SNVEPEDLGVYYCLQYGSVPYTFGAGTKLELKRTVAAP |
| | variable | SVFIFPPSDEQLKSGTASVVCLLNNFYPREAKVQWKVD |
| | domain/human | NALQSGNSQESVTEQDSKDSTYSLSSTLTLSKADYEKH |
| | kappa | KVYACEVTHQGLSSPVTKSFNRGEC |
| 206 | Chimeric Anti- | QVQLKESGPGLVQASETLSLTCTVSGFSLTSYCVNWV |
| | ILT3 rat 24A4 | RQPSGKGPEWLGRFWYDEGKVYNLTLESRLSISGDTS |
| | parental HC | KNQVFLKMNRLRTDDTGTYYCTRDRDTLGITGWFAY |
| | variable | WGQGTLVTVSSASTKGPSVFPLAPSSKSTSGGTAALGC |
| | domain/human | LVKDYFPEPVTVSWNSGALTSGVHTFPAVLQSSGLYSL |
| | IgG4 (S228P) | SSVVTVPSSSLGTQTYICNVNHKPSNTKVDKKVEPKSC |
| | constant domain | DKTHTCPPCPAPEAAGGPSVFLFPPKPKDTLMISRTPE |
| | | VTCVVVSVSHEDPEVKFNWYVDGVEVHNAKTKPREEQ |
| | | YNSTYRVVSVLTVLHQDWLNGKEYKCKVSNKALPAPI |
| | | EKTISKAKGQPREPQVYTLPPSRDELTKNQVSLTCLVK |
| | | GFYPSDIAVEWESNGQPENNYKTTPPVLDSDGSFFLYS |
| | | KLTVDKSRWQQGNVFSCSVMHEALHNHYTQKSLSLS |
| | | PGK |
| 207 | Chimeric Anti- | ETVMTQSPTSLSASIGERVTLNCKASQSVGINVDWYQ |
| | ILT3 rat 24A4 | QTPGQSPKLLIYGSANRHTGVPDRFTGSGFGSGFTLTIS |
| | parental LC | NVEPEDLGVYYCLQYGSVPYTFGPGTKLELKRTVAAP |
| | variable | SVFIFPPSDEQLKSGTASVVCLLNNFYPREAKVQWKVD |
| | domain/human | NALQSGNSQESVTEQDSKDSTYSLSSTLTLSKADYEKH |
| | kappa | KVYACEVTHQGLSSPVTKSFNRGEC |
| 208 | Humanized 52B8 | EVQLVESGGGLVQPGGSLRLSCAASGFTFSNYGMSWV |
| | HC variable | RQAPGKGLEWVATISGGGDYTNYPDSVRGRFTISRDN |
| | domain VH1 | AKNSLYLQMNSLRAEDTAVYYCGRRLWFRSLYYAMD |
| | (M64V)/Human | YWGQGTLVTVSSASTKGPSVFPLAPSSKSTSGGTAALG |
| | IgG1 HC | CLVKDYFPEPVTVSWNSGALTSGVHTFPAVLQSSGLY |
| | (N297A) constant | SLSSVVTVPSSSLGTQTYICNVNHKPSNTKVDKKVEPK |
| | domain | SCDKTHTCPPCPAPELLGGPSVFLFPPKPKDTLMISRTP |
| | | EVTCVVVDVSHEDPEVKFNWYVDGVEVHNAKTKPRE |
| | | EQYASTYRVVSVLTVLHQDWLNGKEYKCKVSNKALP |
| | | APIEKTISKAKGQPREPQVYTLPPSRDELTKNQVSLTCL |
| | | VKGFYPSDIAVEWESNGQPENNYKTTPPVLDSDGSFFL |
| | | YSKLTVDKSRWQQGNVFSCSVMHEALHNHYTQKSLS |
| | | LSPGK |
| 209 | Human IgG1 HC | ASTKGPSVFPLAPSSKSTSGGTAALGCLVKDYFPEPVT |
| | constant domain | VSWNSGALTSGVHTFPAVLQSSGLYSLSSVVTVPSSSL |
| | (N297A) | GTQTYICNVNHKPSNTKVDKKVEPKSCDKTHTCPPCP |
| | | APELLGGPSVFLFPPKPKDTLMISRTPEVTCVVVDVSH |
| | | EDPEVKFNWYVDGVEVHNAKTKPREEQYASTYRVVS |
| | | VLTVLHQDWLNGKEYKCKVSNKALPAPIEKTISKAKG |
| | | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |
| 210 | Chimeric anti- | QVQLKESGPGLVQASETLSLTCTVSGFSLTSYSINWVR |
| | ILT3 40A6 rat VH | QSSGKGPEWMGRFWYDEGIAYNLTLESRLSISGDTSK |
| | /human IgG1 | NQVFLKMNSLRTGDTGTYYCTRDRDTVGITGWFAYW |
| | (N297A) | GQGTLVTVSSASTKGPSVFPLAPSSKSTSGGTAALGCL |
| | | VKDYFPEPVTVSWNSGALTSGVHTFPAVLQSSGLYSLS |
| | | SVVTVPSSSLGTQTYICNVNHKPSNTKVDKKVEPKSCD |
| | | KTHTCPPCPAPELLGGPSVFLFPPKPKDTLMISRTPEVT |
| | | CVVVDVSHEDPEVKFNWYVDGVEVHNAKTKPREEQY |
| | | ASTYRVVSVLTVLHQDWLNGKEYKCKVSNKALPAPIE |
| | | KTISKAKGQPREPQVYTLPPSRDELTKNQVSLTCLVKG |
| | | FYPSDIAVEWESNGQPENNYKTTPPVLDSDGSFFLYSK |
| | | LTVDKSRWQQGNVFSCSVMHEALHNHYTQKSLSLSP |
| | | GK |
| 211 | Chimeric anti- | QVQLKESGPGLVQASETLSLTCTVSGFSLTNYCVNWV |
| | ILT3 16B1 rat VH | RQPSGKGPEWLGRFWFDEGKAYNLTLESRLSISGDTSK |
| | /human IgG1 | NQVFLRMNSLRADDTGTYYCTRDRDTVGITGWFAYW |
| | (N297A) | GQGTLVTVSSASTKGPSVFPLAPSSKSTSGGTAALGCL |
| | | VKDYFPEPVTVSWNSGALTSGVHTFPAVLQSSGLYSLS |
| | | SVVTVPSSSLGTQTYICNVNHKPSNTKVDKKVEPKSCD |
| | | KTHTCPPCPAPELLGGPSVFLFPPKPKDTLMISRTPEVT |
| | | CVVVDVSHEDPEVKFNWYVDGVEVHNAKTKPREEQY |
| | | ASTYRVVSVLTVLHQDWLNGKEYKCKVSNKALPAPIE |
| | | KTISKAKGQPREPQVYTLPPSRDELTKNQVSLTCLVKG |
| | | FYPSDIAVEWESNGQPENNYKTTPPVLDSDGSFFLYSK |
| | | LTVDKSRWQQGNVFSCSVMHEALHNHYTQKSLSLSP |
| | | GK |
| 212 | Chimeric anti- | QVQLQQSGAELMKPGASVKISCKATGYTFRTYWIEWV |
| | ILT3 11D1 mouse | KQRPGHGLEWIGEILPGNGNTHENENFKDKATFTADTS |
| | VH/human IgG1 | SNAAYMQLSSLTSEDSAVYYCVRRLGRGPFDFWGQG |
| | (N297A) | TTLTVSSASTKGPSVFPLAPSSKSTSGGTAALGCLVKD |
| | | YFPEPVTVSWNSGALTSGVHTFPAVLQSSGLYSLSSVV |
| | | TVPSSSLGTQTYICNVNHKPSNTKVDKKVEPKSCDKTH |
| | | TCPPCPAPELLGGPSVFLFPPKPKDTLMISRTPEVTCVV |
| | | VDVSHEDPEVKFNWYVDGVEVHNAKTKPREEQYAST |
| | | YRVVSVLTVLHQDWLNGKEYKCKVSNKALPAPIEKTI |
| | | SKAKGQPREPQVYTLPPSRDELTKNQVSLTCLVKGFYP |
| | | SDIAVEWESNGQPENNYKTTPPVLDSDGSFFLYSKLTV |
| | | DKSRWQQGNVFSCSVMHEALHNHYTQKSLSLSPGK |
| 213 | Chimeric anti- | EVQLVESGGGLVQPGRSMKLSCAASGFTFSNFDMAW |
| | ILT3 17H12 rat | VRQAPTRGLEWVSSITYDGGSTSYRDSVKGRFTISRDN |
| | VH/human IgG1 | AKGTLYLQMDSLRSEDTATYYCTTVESIATISTYFDYW |
| | (N297A) | GQGVMVTVSSASTKGPSVFPLAPSSKSTSGGTAALGCL |
| | | VKDYFPEPVTVSWNSGALTSGVHTFPAVLQSSGLYSLS |
| | | SVVTVPSSSLGTQTYICNVNHKPSNTKVDKKVEPKSCD |
| | | KTHTCPPCPAPELLGGPSVFLFPPKPKDTLMISRTPEVT |
| | | CVVVDVSHEDPEVKFNWYVDGVEVHNAKTKPREEQY |
| | | ASTYRVVSVLTVLHQDWLNGKEYKCKVSNKALPAPIE |
| | | KTISKAKGQPREPQVYTLPPSRDELTKNQVSLTCLVKG |
| | | FYPSDIAVEWESNGQPENNYKTTPPVLDSDGSFFLYSK |
| | | LTVDKSRWQQGNVFSCSVMHEALHNHYTQKSLSLSP |
| | | GK |
| 214 | Chimeric anti- | QVQLKESGPGLVQASETLSLTCTVSGFSLTSYCVNWV |
| | ILT3 37C8 rat VH | RQPSGKGPEWLGRFWYDEGKVYNLTLESRLSISGDTS |
| | /human IgG1 | KNQVFLKMNRLRTDDTGTYYCTRDRDTMGITGWFAY |
| | (N297A) | WGQGTLVTVSSASTKGPSVFPLAPSSKSTSGGTAALGC |
| | | LVKDYFPEPVTVSWNSGALTSGVHTFPAVLQSSGLYSL |
| | | SSVVTVPSSSLGTQTYICNVNHKPSNTKVDKKVEPKSC |
| | | DKTHTCPPCPAPELLGGPSVFLFPPKPKDTLMISRTPEV |
| | | TCVVVDVSHEDPEVKFNWYVDGVEVHNAKTKPREEQ |
| | | YASTYRVVSVLTVLHQDWLNGKEYKCKVSNKALPAPI |
| | | EKTISKAKGQPREPQVYTLPPSRDELTKNQVSLTCLVK |
| | | GFYPSDIAVEWESNGQPENNYKTTPPVLDSDGSFFLYS |
| | | KLTVDKSRWQQGNVFSCSVMHEALHNHYTQKSLSLS |
| | | PGK |
| 215 | Chimeric anti- | QVQMQQSGTELMKPGASMKISCKATGYTFSTYWIQWI |
| | ILT3 1G12 mouse | KQRPGHGLEWIGEILPGSGTTNYNENFKGKATFSADTS |
| | VH/human IgG1 | SNTAYIHLSSLTSEDSAVFYCARRLGRGPFDYWGQGTT |
| | (N297A) | LTVSSASTKGPSVFPLAPSSKSTSGGTAALGCLVKDYF |
| | | PEPVTVSWNSGALTSGVHTFPAVLQSSGLYSLSSVVTV |
| | | PSSSLGTQTYICNVNHKPSNTKVDKKVEPKSCDKTHTC |
| | | PPCPAPELLGGPSVFLFPPKPKDTLMISRTPEVTCVVVD |
| | | VSHEDPEVKFNWYVDGVEVHNAKTKPREEQYASTYR |
| | | VVSVLTVLHQDWLNGKEYKCKVSNKALPAPIEKTISK |
| | | AKGQPREPQVYTLPPSRDELTKNQVSLTCLVKGFYPSD |
| | | IAVEWESNGQPENNYKTTPPVLDSDGSFFLYSKLTVDK |
| | | SRWQQGNVFSCSVMHEALHNHYTQKSLSLSPGK |
| 216 | Chimeric anti- | QVQLKESGPGLVQASETLSLTCTVSGFSLTSYSVNWVR |
| | ILT3 20E4 rat VH | QPSGKGLEWMGRFWYDGGTAYNSTLESRLSISGDTSK |
| | /human IgG1 | NQVFLKMNSLQTDDTGTYYCTRDRDTMGITGWFAYW |
| | (N297A) | GQGTLVTVSPASTKGPSVFPLAPSSKSTSGGTAALGCL |
| | | VKDYFPEPVTVSWNSGALTSGVHTFPAVLQSSGLYSLS |
| | | SVVTVPSSSLGTQTYICNVNHKPSNTKVDKKVEPKSCD |
| | | KTHTCPPCPAPELLGGPSVFLFPPKPKDTLMISRTPEVT |
| | | CVVVDVSHEDPEVKFNWYVDGVEVHNAKTKPREEQY |
| | | ASTYRVVSVLTVLHQDWLNGKEYKCKVSNKALPAPIE |
| | | KTISKAKGQPREPQVYTLPPSRDELTKNQVSLTCLVKG |
| | | FYPSDIAVEWESNGQPENNYKTTPPVLDSDGSFFLYSK |
| | | LTVDKSRWQQGNVFSCSVMHEALHNHYTQKSLSLSP |
| | | GK |
| 217 | Chimeric anti- | QVQLKESGPGLVQASETLSLTCTVSGFSLTSYCVNWV |
| | ILT3 24A4 rat VH | RQPSGKGPEWLGRFWYDEGKVYNLTLESRLSISGDTS |
| | /human IgG1 | KNQVFLKMNRLRTDDTGTYYCTRDRDTLGITGWFAY |
| | (N297A) | WGQGTLVTVSSASTKGPSVFPLAPSSKSTSGGTAALGC |
| | | LVKDYFPEPVTVSWNSGALTSGVHTFPAVLQSSGLYSL |
| | | SSVVTVPSSSLGTQTYICNVNHKPSNTKVDKKVEPKSC |
| | | DKTHTCPPCPAPELLGGPSVFLFPPKPKDTLMISRTPEV |
| | | TCVVVDVSHEDPEVKFNWYVDGVEVHNAKTKPREEQ |
| | | YASTYRVVSVLTVLHQDWLNGKEYKCKVSNKALPAPI |
| | | EKTISKAKGQPREPQVYTLPPSRDELTKNQVSLTCLVK |
| | | GFYPSDIAVEWESNGQPENNYKTTPPVLDSDGSFFLYS |
| | | KLTVDKSRWQQGNVFSCSVMHEALHNHYTQKSLSLS |
| | | PGK |
| 218 | Chimeric anti- | QVQLKESGPGLVQASETLSLTCTVSGFSLTSYSINWVR |
| | ILT3 40A6 rat VH/ | QSSGKGPEWMGRFWYDEGIAYNLTLESRLSISGDTSK |
| | human IgG1 | NQVFLKMNSLRTGDTGTYYCTRDRDTVGITGWFAYW |
| | (N297A) | GQGTLVTVSSASTKGPSVFPLAPSSKSTSGGTAALGCL |
| | | VKDYFPEPVTVSWNSGALTSGVHTFPAVLQSSGLYSLS |
| | | SVVTVPSSSLGTQTYICNVNHKPSNTKVDKKVEPKSCD |
| | | KTHTCPPCPAPELLGGPSVFLFPPKPKDTLMISRTPEVT |
| | | CVVVDVSHEDPEVKFNWYVDGVEVHNAKTKPREEQY |
| | | ASTYRVVSVLTVLHQDWLNGKEYKCKVSNKALPAPIE |
| | | KTISKAKGQPREPQVYTLPPSRDELTKNQVSLTCLVKG |
| | | FYPSDIAVEWESNGQPENNYKTTPPVLDSDGSFFLYSK |
| | | LTVDKSRWQQGNVFSCSVMHEALHNHYTQKSLSLSP |
| | | GK |
| 219 | Residues after LC- | FGXG |
| | CDR3 | |
| | Xaa is any amino | |
| | acid | |
| 220 | Residues before | CXXX |
| | HC-CDR1 | |
| | Xaa is any amino | |
| | acid | |
| 221 | Residues before | LEWIG |
| | HC-CDR1 | |
| 222 | Residues after HC- | WGXG |
| | CDR3 | |
| | Xaa is any residue | |
| 223 | Human IgG1 HC | ASTKGPSVFPLAPSSKSTSGGTAALGCLVKDYFPEPVT |
| | constant domain | VSWNSGALTSGVHTFPAVLQSSGLYSLSSVVTVPSSSL |
| | Residue 148 | GTQTYICNVNHKPSNTKVDKKVEPKSCDKTHTCPPCP |
| | corresponds with | APELLGGPSVFLFPPKPKDTLMISRTPEVTCVVVAVSH |
| | N297A, residue | EDPEVKFNWYVDGVEVHNAKTKPREEQYASTYRVVS |
| | 180 corresponds | VLTVLHQDWLNGKEYKCKVSNKALPAPIEKTISKAKG |
| | with D265A | QPREPQVYTLPPSRDELTKNQVSLTCLVKGFYPSDIAV |
| | | EWESNGQPENNYKTTPPVLDSDGSFFLYSKLTVDKSR |
| | | WQQGNVFSCSVMHEALHNHYTQKSLSLSPGK |

In specific embodiments, the methods and uses of the present invention provides the anti-ILT3 antibodies shown in Table 6 below. With the exception of those antibodies comprising a replacement of the tryptophan residue at position 101 of the V_{H}, the antibodies disclosed herein bind human ILT3.

TABLE 6
Exemplary anti-ILT3 antibodies

| | | SEQ ID NO: | |
|---|---|---|---|
| | | Heavy | Light |
| mAb No. | Description | Chain | Chain |
| 1 | Humanized anti-ILT3 mAb (52B8 VH1/VL1) IgG4 | 140 | 149 |
| | S228P/Kappa | | |
| 2 | Humanized anti-ILT3 mAb (52B8 VH1/VL2) IgG4 | 140 | 150 |
| | S228P/Kappa | | |
| 3 | Humanized anti-ILT3 mAb (52B8 VH1/VL3) IgG4 | 140 | 151 |
| | S228P/Kappa | | |
| 4 | Humanized anti-ILT3 mAb (52B8 VH1/VL4) IgG4 | 140 | 152 |
| | S228P/Kappa | | |
| 5 | Humanized anti-ILT3 mAb (52B8 VH2/VL1) IgG4 | 146 | 149 |
| | S228P/Kappa | | |
| 6 | Humanized anti-ILT3 mAb (52B8 VH2/VL2) IgG4 | 146 | 150 |
| | S228P/Kappa | | |
| 7 | Humanized anti-ILT3 mAb (52B8 VH2/VL3) IgG4 | 146 | 151 |
| | S228P/Kappa | | |
| 8 | Humanized anti-ILT3 mAb (52B8 VH2/VL4) IgG4 | 146 | 152 |
| | S228P/Kappa | | |
| 9 | Humanized anti-ILT3 mAb (52B8 VH1 M64V/VL1) | 141 | 149 |
| | IgG4 S228P/Kappa | | |
| 10 | Humanized anti-ILT3 mAb (52B8 VH1 M64V/VL2) | 141 | 150 |
| | IgG4 S228P/Kappa | | |
| 11 | Humanized anti-ILT3 mAb (52B8 VH1 M64V/VL3) | 141 | 151 |
| | IgG4 S228P/Kappa | | |
| 12 | Humanized anti-ILT3 mAb (52B8 VH1 M64V/VL4) | 141 | 152 |
| | IgG4 S228P/Kappa | | |
| 13 | Humanized anti-ILT3 mAb (52B8 VH2 M64V/VL1) | 147 | 149 |
| | IgG4 S228P/Kappa | | |
| 14 | Humanized anti-ILT3 mAb (52B8 VH2 M64V/VL2) | 147 | 150 |
| | IgG4 S228P/Kappa | | |
| 15 | Humanized anti-ILT3 mAb (52B8 VH2 M64V/VL3) | 147 | 151 |
| | IgG4 S228P/Kappa | | |
| 16 | Humanized anti-ILT3 mAb (52B8 VH2 M64V/VL4) | 147 | 152 |
| | IgG4 S228P/Kappa | | |
| 17 | Humanized anti-ILT3 mAb (52B8 VH1 M64L/VL1) | 142 | 149 |
| | IgG4 S228P/Kappa | | |
| 18 | Humanized anti-ILT3 mAb (52B8 VH1 M64L/VL2) | 142 | 150 |
| | IgG4 S228P/Kappa | | |
| 19 | Humanized anti-ILT3 mAb (52B8 VH1 M64L/VL3) | 142 | 151 |
| | IgG4 S228P/Kappa | | |
| 20 | Humanized anti-ILT3 mAb (52B8 VH1 M64L/VL4) | 142 | 153 |
| | IgG4 S228P/Kappa | | |
| 21 | Humanized anti-ILT3 mAb (52B8 VH2 M64L/VL1) | 148 | 149 |
| | IgG4 S228P/Kappa | | |
| 22 | Humanized anti-ILT3 mAb (52B8 VH2 M64L/VL2) | 148 | 150 |
| | IgG4 S228P/Kappa | | |
| 23 | Humanized anti-ILT3 mAb (52B8 VH2 M64L/VL3) | 148 | 151 |
| | IgG4 S228P/Kappa | | |
| 24 | Humanized anti-ILT3 mAb (52B8 VH2 M64L/VL4) | 148 | 152 |
| | IgG4 S228P/Kappa | | |
| 25 | Humanized anti-ILT3 mAb ((52B8 VH1 M64V/VL2) | 167 | 150 |
| | L234A L235A D265S) IgG1/Kappa | | |
| 26 | Humanized anti-ILT3 mAb ((52B8 VH1 M64V/VL5) | 167 | 153 |
| | L234A L235A D265S) IgG1/Kappa | | |
| 27 | Humanized anti-ILT3 mAb ((52B8 VH1 M64V/VL6) | 167 | 154 |
| | L234A L235A D265S) IgG1/Kappa | | |
| 28 | Humanized anti-ILT3 mAb ((52B8 VH1 M64V/VL7) | 157 | 155 |
| | L234A L235A D265S) IgG1/Kappa | | |
| 29 | Humanized anti-ILT3 mAb ((52B8 VH1 M64V/VL8) | 167 | 156 |
| | L234A L235A D265S) IgG1/Kappa | | |
| 30 | Humanized anti-ILT3 mAb (52B8 VH1 M64V/VL5) | 141 | 153 |
| | IgG4 S228P/Kappa | | |
| 31 | Humanized anti-ILT3 mAb (52B8 VH1 M64V/VL6) | 141 | 154 |
| | IgG4 S228P/Kappa | | |
| 32 | Humanized anti-ILT3 mAb (52B8 VH1 M64V/VL7) | 141 | 155 |
| | IgG4 S228P/Kappa | | |
| 33 | Humanized anti-ILT3 mAb (52B8 VH1 M64V/VL8) | 141 | 156 |
| | IgG4 S228P/Kappa | | |
| 34 | Humanized anti-ILT3 mAb (52B8 VH1 M64V W101F/ | 143 | 150 |
| | VL2) IgG4 S228P/Kappa | | |
| 35 | Humanized anti-ILT3 mAb (52B8 VH1 M64V W101Y/ | 144 | 150 |
| | VL2) IgG4 S228P/Kappa | | |
| 36 | Humanized anti-ILT3 mAb (52B8 VH1 M64V W101Q/ | 145 | 150 |
| | VL2) IgG4 S228P/Kappa | | |
| 37 | Humanized anti-ILT3 mAb ((52B8 VH1 M64V W101F/ | 143 | 150 |
| | VL2) L234A L235A D265S) IgG1/Kappa | | |
| 38 | Humanized anti-ILT3 mAb ((52B8 VH1 M64V W101Y/ | 144 | 150 |
| | VL2) L234A L235A D265S) IgG1/Kappa | | |
| 39 | Humanized anti-ILT3 mAb ((52B8 VH1 M64V W101Q/ | 145 | 150 |
| | VL2) L234A L235A D265S) IgG1/Kappa | | |
| 40 | Humanized anti-ILT3 mAb (52B8 VH1 M64V/VL2 | 141 | 157 |
| | S35A) IgG4 S228P/Kappa | | |
| 41 | Humanized anti-ILT3 mAb (52B8 VH1 M64V/VL2 | 141 | 158 |
| | S35N) IgG4 S228P/Kappa | | |
| 42 | Humanized anti-ILT3 mAb (52B8 VH1 M64V/VL2 | 141 | 159 |
| | N34Q) IgG4 S228P/Kappa | | |
| 43 | Humanized anti-ILT3 mAb (52B8 VH1 M64V/VL2 | 141 | 160 |
| | N34D) IgG4 S228P/Kappa | | |
| 44 | Humanized anti-ILT3 mAb (52B8 VH1 M64V/VL5 | 141 | 161 |
| | S35A) IgG4 S228P/Kappa | | |
| 45 | Humanized anti-ILT3 mAb (52B8 VH1 M64V/VL5 | 141 | 162 |
| | S35N) IgG4 S228P/Kappa | | |
| 46 | Humanized anti-ILT3 mAb (52B8 VH1 M64V/VL5 | 141 | 163 |
| | N34Q) IgG4 S228P/Kappa | | |
| 47 | Humanized anti-ILT3 mAb (52B8 VH1 M64V/VL5 | 141 | 164 |
| | N34D) IgG4 S228P/Kappa | | |
| 48 | Humanized anti-ILT3 mAb (52B8 VH1 M64V W101F/ | 143 | 153 |
| | VL5) IgG4 S228P/Kappa | | |
| 49 | Humanized anti-ILT3 mAb (52B8 VH1 M64V W101Y/ | 144 | 153 |
| | VL5) IgG4 S228P/Kappa | | |
| 50 | Humanized anti-ILT3 mAb (52B8 VH1 M64V W101Q/ | 145 | 153 |
| | VL5) IgG4 S228P/Kappa | | |
| 51 | Humanized anti-ILT3 mAb (52B8 VH1 M64V W101F/ | 143 | 161 |
| | VL5 S35A) IgG4 S228P/Kappa | | |
| 52 | Humanized anti-ILT3 mAb (52B8 VH1 M64V W101F/ | 143 | 161 |
| | VL5 S35N) IgG4 S228P/Kappa | | |
| 53 | Humanized anti-ILT3 mAb (52B8 VH1 M64V W101F/ | 143 | 163 |
| | VL5 N34Q) IgG4 S228P/Kappa | | |
| 54 | Humanized anti-ILT3 mAb (52B8 VH1 M64V W101F/ | 143 | 164 |
| | VL5 N34D) IgG4 S228P/Kappa | | |
| 55 | Humanized anti-ILT3 mAb (52B8 VH1 M64V W101Y/ | 144 | 161 |
| | VL5 S35A) IgG4 S228P/Kappa | | |
| 56 | Humanized anti-ILT3 mAb (52B8 VH1 M64V W101Y/ | 144 | 162 |
| | VL5 S35N) IgG4 S228P/Kappa | | |
| 57 | Humanized anti-ILT3 mAb (52B8 VH1 M64V W101Y/ | 144 | 163 |
| | VL5 N34Q) IgG4 S228P/Kappa | | |
| 58 | Humanized anti-ILT3 mAb (52B8 VH1 M64V W101Y/ | 144 | 164 |
| | VL5 N34D) IgG4 S228P/Kappa | | |
| 59 | Humanized anti-ILT3 mAb (52B8 VH1 M64V W101Q/ | 145 | 161 |
| | VL5 S35A) IgG4 S228P/Kappa | | |
| 60 | Humanized anti-ILT3 mAb (52B8 VH1 M64V W101Q/ | 145 | 162 |
| | VL5 S35N) IgG4 S228P/Kappa | | |
| 61 | Humanized anti-ILT3 mAb (52B8 VH1 M64V W101Q/ | 145 | 163 |
| | VL5 N34Q) IgG4 S228P/Kappa | | |
| 62 | Humanized anti-ILT3 mAb (52B8 VH1 M64V W101Q/ | 145 | 164 |
| | VL5 N34D) IgG4 S228P/Kappa | | |
| 63 | Humanized anti-ILT3 mAb (52B8 VH1 M64V/VL1 | 208 | 124 |
| | N34Q) IgG1 N297A/Kappa | | |
| 64 | Humanized anti-ILT3 mAb (52B8 VH1 M64V/VL2 | 208 | 125 |
| | IgG1 N297A/Kappa | | |
| 65 | Humanized anti-ILT3 mAb (52B8 VH1 M64V/VL2 | 208 | 159 |
| | N34Q) IgGI N297A/Kappa | | |
| 66 | Humanized anti-ILT3 mAb (52B8 VH1 M64V/VL3 | 208 | 126 |
| | N34Q) IgG1 N297A/Kappa | | |
| 67 | Humanized anti-ILT3 mAb (52B8 VH1 M64V/VL4 | 208 | 127 |
| | N34Q) IgG1 N297A/Kappa | | |
| 68 | Humanized anti-ILT3 mAb (52B8 VH1 M64V/VL5 | 209 | 128 |
| | IgG1 N297A/Kappa | | |
| 69 | Humanized anti-ILT3 mAb (52B8 VH1 M64V/VL5 | 208 | 163 |
| | N34Q) IgG1 N297A/Kappa | | |
| 70 | Humanized anti-ILT3 mAb (52B8 VH1 M64V/VL6 | 208 | 129 |
| | N34Q) IgG1 N297A/Kappa | | |
| 71 | Humanized anti-ILT3 mAb (52B8 VH1 M64V/VL7 | 208 | 130 |
| | N34Q) IgG1 N297A/Kappa | | |
| 72 | Humanized anti-ILT3 mAb (52B8 VH1 M64V/VL8 | 208 | 131 |
| | N34Q) IgG1 N297A/Kappa | | |
| 73 | Chimeric anti-ILT3 52B8 mouse VH/human | 111 | 114 |
| | IgG4 (S228P):mouse VL/human Kappa | | |
| 74 | Chimeric anti-ILT3 52B8 mouse VH M64V/human | 112 | 114 |
| | IgG4 (S228P):mouse VL/human Kappa | | |
| 75 | Chimeric anti-ILT3 52B8 mouse VH M64L/human | 113 | 114 |
| | IgG4 (S228P):mouse VL/human Kappa | | |
| 76 | Chimeric anti-ILT3 52B8 mouse VH/human | Residues | 114 |
| | IgG1 (N297A):mouse VL/human Kappa | 1-122 of | |
| | | SEQ ID | |
| | | NO: 111 | |
| | | And | |
| | | SEQ ID | |
| | | NO: 209 | |
| 77 | Chimeric anti-ILT3 52B8 mouse VH M64V/human | Residues | 114 |
| | IgG1 (N297A):mouse VL/human Kappa | 1-122 of | |
| | | SEQ ID | |
| | | NO: 112 | |
| | | And | |
| | | SEQ ID | |
| | | NO: 209 | |
| 78 | Chimeric anti-ILT3 52B8 mouse VH/human | Residues | 114 |
| | IgG1:mouse VL/human Kappa | 1-122 of | |
| | | SEQ ID | |
| | | NO: 111 | |
| | | And | |
| | | SEQ ID | |
| | | NO: 9 | |
| 79 | Chimeric anti-ILT3 52B8 mouse VH M64V/human | Residues | 114 |
| | IgG1:mouse VL/human Kappa | 1-122 of | |
| | | SEQ ID | |
| | | NO: 112 | |
| | | And | |
| | | SEQ ID | |
| | | NO: 9 | |
| 80 | Chimeric anti-ILT3 40A6 rat VH/human IgG4 | 192 | 193 |
| | (S228P):rat VL/human Kappa | | |
| 81 | Chimeric anti-ILT3 16B1 rat VH/human IgG4 | 194 | 195 |
| | (S228P):rat VL/human Kappa | | |
| 82 | Chimeric anti-ILT3 11D1 mouse VH/human IgG4 | 196 | 197 |
| | (S228P):mouse VL/human Kappa | | |
| 83 | Chimeric anti-ILT3 17H12 rat VH/human IgG4 | 198 | 199 |
| | (S228P):rat VL/human Kappa | | |
| 84 | Chimeric anti-ILT3 37C8 rat VH/human IgG4 | 200 | 201 |
| | (S228P):rat VL/human Kappa | | |
| 85 | Chimeric anti-ILT3 1G12 mouse VH/human IgG4 | 201 | 203 |
| | (S228P):mouse VL/human Kappa | | |
| 86 | Chimeric anti-ILT3 20E4 rat VH/human IgG4 | 204 | 205 |
| | (S228P):rat VL/human Kappa | | |
| 87 | Chimeric anti-ILT3 24A4 rat VH/human IgG4 | 206 | 207 |
| | (S228P):rat VL/human Kappa | | |
| 88 | Chimeric anti-ILT3 40A6 rat VH/human IgG1 | 210 | 193 |
| | (N297A):rat VL/human Kappa | | |
| 89 | Chimeric anti-ILT3 16B1 rat VH/human IgG1 | 211 | 195 |
| | (N297A):rat VL/human Kappa | | |
| 90 | Chimeric anti-ILT3 11D1 mouse VH/human IgG1 | 212 | 197 |
| | (N297A):mouse VL/human Kappa | | |
| 91 | Chimeric anti-ILT3 17H12 rat VH/human IgG1 | 213 | 199 |
| | (N297A):rat VL/human Kappa | | |
| 92 | Chimeric anti-ILT3 37C8 rat VH/human IgG1 | 214 | 201 |
| | (N297A):rat VL/human Kappa | | |
| 93 | Chimeric anti-ILT3 1G12 mouse VH/human IgG1 | 215 | 203 |
| | (N297A):mouse VL/human Kappa | | |
| 94 | Chimeric anti-ILT3 20E4 rat VH/human IgG1 | 216 | 205 |
| | (N297A):rat VL/human Kappa | | |
| 95 | Chimeric anti-ILT3 24A4 rat VH/human IgG1 | 217 | 207 |
| | (N297A):rat VL/human Kappa | | |
| 96 | Chimeric anti-ILT3 40A6 rat VH/human IgG1 | 215 | 193 |
| | (N297A):rat VL/human Kappa | | |

In particular embodiments of the invention, the anti-ILT3 antigen binding protein or fragment is a human or humanized anti-ILT3 antibody or antigen binding fragment or a chimeric anti-ILT3 antibody or antigen binding fragment that comprises HC-CDR1, HC-CDR2, HC-CDR3, LC-CDR1, LC-CDR2, and LC-CDR3 of an anti-ILT3 antibody molecule disclosed herein or in Table 7 below.

TABLE 7

| | | SEQ | | SEQ | | SEQ |
|---|---|---|---|---|---|---|
| | | ID | | ID | | ID |
| | | NO. | | NO. | | NO. |
| mAb | HC-CDR1 | | HC-CDR2 | | HC-CDR3 | |
| 52B8 | NYGMS | 15 | TISGGGDYT | 18 | RLWFRSLY | 21 |
| | | | NYPDSVRG | | YAMDY | |
| 40A6 | SYSIN | 45 | RFWYDEGIA | 46 | DRDTVGITG | 47 |
| | | | YNLTLES | | WFAY | |
| 16B1 | NYCVN | 53 | RFWFDEGKA | 54 | DRDTVGITG | 55 |
| | | | YNLTLES | | WFAY | |
| 11D1 | TYWIE | 61 | EILPGNGNT | 62 | RRLGRGPFDF | 63 |
| | | | HENENFKD | | | |
| 17H12 | NFDMA | 69 | SITYDGGST | 70 | VESIATIS | 71 |
| | | | SYRDSVKG | | TYFDY | |
| 37C8 | SYCVN | 77 | RFWYDEGKV | 78 | DRDTMGIT | 79 |
| | | | YNLTLES | | GWFAY | |
| 1G12 | TYWIQ | 85 | EILPGSGTT | 86 | RLGRGPFDY | 87 |
| | | | NYNENFKG | | | |
| 20E4 | SYSVN | 93 | RFWYDGGTA | 94 | DRDTMGITG | 95 |
| | | | YNSTLES | | WFAY | |
| 24A4 | SYCVN | 101 | RFWYDEGKV | 102 | DRDTLGITGW | 103 |
| | | | YNLTLES | | FAY | |
| mAb | LC-CDR1 | | LC-CDR2 | | LC-CDR3 | |
| 52B8 | RASEKVDS | 39 | LTSNLDS | 41 | QQNNEDPYT | 42 |
| | FGQSFMH | | | | | |
| 40A6 | KASQSVGV | 48 | GSANRHT | 49 | LQYGSVPYT | 50 |
| | NVD | | | | | |
| 16B1 | KASQSVG | 56 | GSANRHT | 57 | LQYGSVPYT | 58 |
| | INVD | | | | | |
| 11D1 | KASQDIN | 64 | YTSTLQS | 65 | LQYANPLPT | 66 |
| | EYIG | | | | | |
| 17H12 | RASQSVSM | 72 | RASDLAS | 73 | QQTRKSPPT | 74 |
| | SRYDLIH | | | | | |
| 37C8 | KASQSVG | 80 | GSANRHT | 81 | LQYGSVPYT | 82 |
| | INVD | | | | | |
| 1G12 | EASQDIN | 88 | YASILQP | 89 | LQYDNLLPT | 90 |
| | KHID | | | | | |
| 20E4 | KASQSV | 96 | GSANRHT | 97 | LQYGSVPYT | 98 |
| | GVNVD | | | | | |
| 24A4 | KASQSVG | 104 | GSANRHT | 105 | LQYGSVPYT | 106 |
| | INVD | | | | | |

### Anti-PD-1 Antigen Binding Proteins and Antigen Binding Fragments Useful in the Invention

An “anti-PD-1 antigen binding protein or antigen binding fragment” useful in the any of the methods, compositions and uses of the present invention include monoclonal antibodies (mAb), or antigen binding fragments thereof, which specifically bind to human PD-1. Alternative names or synonyms for PD-1 and its ligands include: PDCD1, PD1, CD279 and SLEB2 for PD-1; PDCDIL1, PDL1, B7H1, B7-4, CD274 and B7-H for PD-L1; and PDCD1L2, PDL2, B7-DC, Btdc and CD273 for PD-L2. In any of the methods, compositions and uses of the present invention in which a human individual is being treated, the PD-1 antigen binding protein or antigen binding fragment is a PD-1 antagonist that blocks binding of human PD-L1 to human PD-1, or blocks binding of both human PD-L1 and PD-1.2 to human PD-1 Human PD-1 amino acid sequences can be found in NCBI Locus No: NP_005009. Human PD-L1 and PD-L2 amino acid sequences can be found in NCBI Locus No.: NP_054862 and NP_079515, respectively. An anti-PD-1 antibody may be a human antibody, a humanized antibody or a chimeric antibody, and may include a human constant region. In some embodiments the human constant region is selected from the group consisting of IgG1. IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

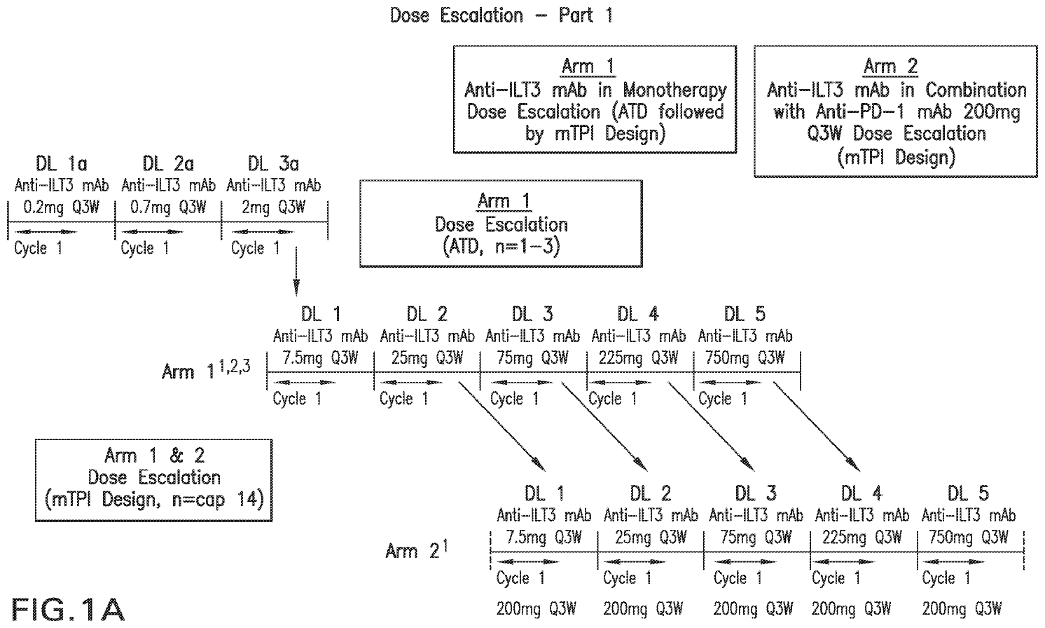

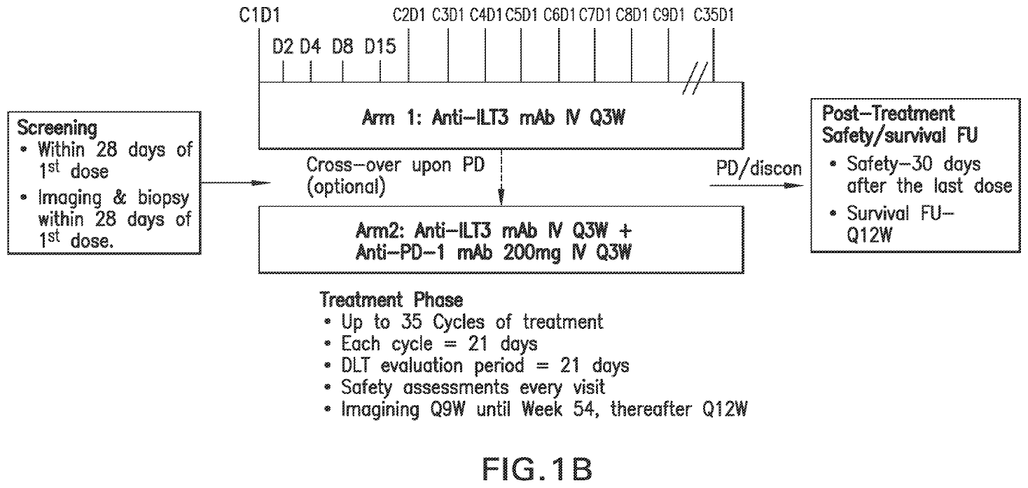

Examples of mAbs that bind to human PD-1, useful in the methods and uses of the invention, are described in U.S. Pat. Nos. 7,521,051, 8,008,449, and 8,354,509. Specific anti-human PD-1 mAbs useful as a PD-1 antagonist in the methods, compositions, and uses of the present invention include: pembrolizumab (formerly known as MK-3475, SCH 900475 and lambrolizumab), a humanized IgG4 mAb with the structure described in WHO Drug Information, Vol. 27, No. 2, pages 161-162 (2013) and which comprises the heavy and light chain amino acid sequences shown in FIG. 1, and the humanized antibodies h409A11, h409A16 and h409A17, which are described in WO 2008/156712 and in Table 8.

In some embodiments of the methods, compositions, kits and uses of the present invention, the anti-PD-1 antigen binding protein, antibody, or antigen binding fragment, comprises: (a) light chain CDRs comprising a sequence of amino acids as set forth in SEQ ID NOs: 224, 225, and 226 and heavy chain CDRs comprising a sequence of amino acids as set forth in SEQ ID NOs: 227, 228, and 229; or (b) light chain CDRs comprising a sequence of amino acids as set forth in SEQ ID NOs: 230, 231, and 232 and heavy chain CDRs comprising a sequence of amino acids as set forth in SEQ ID NOs: 233, 234, and 235. In some embodiments, the anti-PD-1 antigen binding protein, antibody or antigen binding fragment is a human antibody. In other embodiments, the anti-PD-1 antigen binding protein, antibody or antigen binding fragment is a humanized antibody. In other embodiments, the anti-PD-1 antigen binding protein, antibody or antigen binding fragment is a chimeric antibody. In specific embodiments, the anti-PD-1 antigen binding protein, antibody or antigen binding fragment is a monoclonal antibody.

In other embodiments of the methods, compositions, and uses of the present invention, the anti-PD-1 antigen binding protein, antibody, or antigen binding fragment, specifically binds to human PD-1 and comprises (a) a heavy chain variable region comprising an amino acid sequence as set forth in SEQ ID NO: 236, or a variant thereof, and (b) a light chain variable region comprising an amino acid sequence selected from the group consisting of SEQ ID NO: 237 or a variant thereof; SEQ ID NO: 238 or a variant thereof; and SEQ ID NO: 239 or a variant thereof.

In another embodiment of the methods, compositions, and uses of the present invention, the anti-PD-1 antigen binding protein or antigen binding fragment is a monoclonal antibody which specifically binds to human PD-1 and comprises (a) a heavy chain comprising or consisting of a sequence of amino acids as set forth in SEQ ID NO: 240, or a variant thereof; and (b) a light chain comprising or consisting of a sequence of amino acids as set forth in SEQ ID NO: 241, or a variant thereof; SEQ ID NO: 242, or a variant thereof; or SEQ ID NO: 243, or a variant thereof.

In yet another embodiment of the methods, compositions and uses of the invention, the anti-PD-1 antigen binding protein or antigen binding fragment is a monoclonal antibody which specifically binds to human PD-1 and comprises (a) a heavy chain comprising or consisting of a sequence of amino acids as set forth in SEQ ID NO: 240 and (b) a light chain comprising or consisting of a sequence of amino acids as set forth in SEQ ID NO: 241.

In some embodiments of the methods, compositions, kits and uses of the present invention, the anti-PD-1 antigen binding protein, antibody, or antigen binding fragment, comprises light chain CDRs comprising a sequence of amino acids as set forth in SEQ ID NOs: 244, 245, and 246; and heavy chain CDRs comprising a sequence of amino acids as set forth in SEQ ID NOs: 249, 250, and 251. In some embodiments, the anti-PD-1 antigen binding protein, antibody or antigen binding fragment is a human antibody. In other embodiments, the anti-PD-1 antigen binding protein, antibody or antigen binding fragment is a humanized antibody. In other embodiments, the anti-PD-1 antigen binding protein, antibody or antigen binding fragment is a chimeric antibody. In specific embodiments, the anti-PD-1 antigen binding protein, antibody or antigen binding fragment is a monoclonal antibody.

In other embodiments of the methods, compositions, and uses of the present invention, the anti-PD-1 antigen binding protein, antibody, or antigen binding fragment, specifically binds to human PD-1 and comprises (a) a heavy chain variable region comprising an amino acid sequence as set forth in SEQ ID NO: 252, or a variant thereof, and (b) a light chain variable region comprising an amino acid sequence selected from the group consisting of SEQ ID NO: 247 or a variant thereof.

In another embodiment of the methods, compositions, and uses of the present invention, the anti-PD-1 antigen binding protein or antigen binding fragment is a monoclonal antibody which specifically binds to human PD-1 and comprises (a) a heavy chain comprising or consisting of a sequence of amino acids as set forth in SEQ ID NO: 253, or a variant thereof; and (b) a light chain comprising or consisting of a sequence of amino acids as set forth in SEQ ID NO: 248.

Table 8 and Table 9 below provides a list of the amino acid sequences of exemplary anti-PD-1 mAbs for use in the methods, compositions, kits and uses of the present invention.

TABLE 8
Exemplary anti-human PD-1 antibodies
A. Comprises light and heavy chain CDRs of hPD-1.09A in
WO2008/156712 (light and heavy chain CDRs of pembrolizumab)

| | |
|---|---|
| CDRL1 | RASKGVSTSGYSYLH |
| | SEQ ID NO: 224 |
| CDRL2 | LASYLES |
| | SEQ ID NO: 225 |
| CDRL3 | QHSRDLPLT |
| | SEQ ID NO: 226 |
| CDRH1 | NYYMY |
| | SEQ ID NO: 227 |
| CDRH2 | GINPSNGGTNFNEKFKN |
| | SEQ ID NO: 228 |
| CDRH3 | RDYRFDMGFDY |
| | SEQ ID NO: 229 |

B. Comprises light and heavy chain CDRs of hPD-1.08A
in WO2008/156712

| | |
|---|---|
| CDRL1 | RASKSVSTSGFSYLH |
| | SEQ ID NO: 230 |
| CDRL2 | LASNLES |
| | SEQ ID NO: 231 |
| CDRL3 | QHSWELPLT |
| | SEQ ID NO: 232 |
| CDRH1 | SYYLY |
| | SEQ ID NO: 233 |
| CDRH2 | GVNPSNGGTNFSEKFKS |
| | SEQ ID NO: 234 |
| CDRH3 | RDSNYDGGFDY |
| | SEQ ID NO: 235 |

C. Comprises the mature h109A heavy chain variable
(VH) region and one of the mature K09A
light chain variable (VL) regions in WO 2008/156712

| | |
|---|---|
| Heavy chain VH | QVQLVQSGVEVKKPGASVKVSCKASGYTFT |
| | NYYMYWVRQAPGQGLEWMGGINPSNGGTNF |
| | NEKFKNRVTLTTDSSTTTAYMELKSLQFDD |
| | TAVYYCARRDYRFDMGFDYWGQGTTVTVSS |
| | SEQ ID NO: 236 (VH of pembrolizumab) |
| Light chain VL | EIVLTQSPATLSLSPGERATLSCRASKGVS |
| | TSGYSYLHWYQQKPGQAPRLLIYLASYLES |
| | GVPARFSGSGSGTDFTLTISSLEPEDFAVY |
| | YCQHSRDLPLTFGGGTKVEIK |
| | SEQ ID NO: 237 (VL of pembrolizumab) or |
| | EIVLTQSPLSLPVTPGEPASISCRASKGVS |
| | TSGYSYLHWYLQKPGQSPQLLIYLASYLES |
| | GVPDRFSGSGSGTDFTLKISRVEAEDVGVY |
| | YCQHSRDLPLTFGQGTKLEIK |
| | (SEQ ID NO: 238) or |
| | DIVMTQTPLSLPVTPGEPASISCRASKGVS |
| | TSGYSYLHWYLQKPGQSPQLLIYLASYLES |
| | GVPDRFSGSGSGTAFTLKISRVEAEDVGLY |
| | YCQHSRDLPLTFGQGTKLEIK |
| | SEQ ID NO: 239 |
| Heavy chain | QVQLVQSGVEVKKPGASVKVSCKASGYTFT |
| | NYYMYWVRQAPGQGLEWMGGINPSNGGTNF |
| | NEKFKNRVTLTTDSSTTTAYMELKSLQFDD |
| | TAVYYCARRDYRFDMGFDYWGQGTTVTVSS |
| | ASTKGPSVFPLAPCSRSTSESTAALGCLVK |
| | DYFPEPVTVSWNSGALTSGVHTFPAVLQSS |
| | GLYSLSSVVTVPSSSLGTKTYTCNVDHKPS |
| | NTKVDKRVESKYGPPCPPCPAPEFLGGPSV |
| | FLFPPKPKDTLMISRTPEVTCVVVDVSQED |
| | PEVQFNWYVDGVEVHNAKTKPREEQFNSTY |
| | RVVSVLTVLHQDWLNGKEYKCKVSNKGLPS |
| | SIEKTISKAKGQPREPQVYTLPPSQEEMTK |
| | NQVSLTCLVKGFYPSDIAVEWESNGQPENN |
| | YKTTPPVLDSDGSFFLYSRLTVDKSRWQEG |
| | NVFSCSVMHEALHNHYTQKSLSLSLGK |
| | SEQ ID NO: 240 |
| | (heavy chain of pembrolizumab) |
| Light chain | EIVLTQSPATLSLSPGERATLSCRASKGVS |
| | TSGYSYLHWYQQKPGQAPRLLIYLASYLES |
| | GVPARFSGSGSGTDFTLTISSLEPEDFAVY |
| | YCQHSRDLPLTFGGGTKVEIKRTVAAPSVF |
| | IFPPSDEQLKSGTASVVCLLNNFYPREAKV |
| | QWKVDNALQSGNSQESVTEQDSKDSTYSLS |
| | STLTLSKADYEKHKVYACEVTHQGLSSPVT |
| | KSFNRGEC |
| | SEQ ID NO: 241 |
| | (light chain of pembrolizumab) or |
| | EIVLTQSPLSLPVTPGEPASISCRASKGVS |
| | TSGYSYLHWYLQKPGQSPQLLIYLASYLES |
| | GVPDRFSGSGSGTDFTLKISRVEAEDVGVY |
| | YCQHSRDLPLTFGQGTKLEIKRTVAAPSVF |
| | IFPPSDEQLKSGTASVVCLLNNFYPREAKV |
| | QWKVDNALQSGNSQESVTEQDSKDSTYSLS |
| | STLTLSKADYEKHKVYACEVTHQGLSSPVT |
| | KSFNRGEC |
| | SEQ ID NO: 242 or |
| | DIVMTQTPLSLPVTPGEPASISCRASKGVS |
| | TSGYSYLHWYLQKPGQSPQLLIYLASYLES |
| | GVPDRFSGSGSGTAFTLKISRVEAEDVGLY |
| | YCQHSRDLPLTFGQGTKLEIKRTVAAPSVF |
| | IFPPSDEQLKSGTASVVCLLNNFYPREAKV |
| | QWKVDNALQSGNSQESVTEQDSKDSTYSLS |
| | STLTLSKADYEKHKVYACEVTHQGLSSPVT |
| | KSFNRGEC |
| | SEQ ID NO: 243 |

TABLE 9
Nivolumab Heavy and Light Chains

| | |
|---|---|
| Nivolumab Light Chain | |
| CDR1 | RASQSVSSYLA |
| | (SEQ ID NO: 244) |
| CDR2 | DASNRAT |
| | (SEQ ID NO: 245) |
| CDR3 | QQSSNWPRT |
| | (SEQ ID NO: 246) |
| Variable | EIVLTQSPATLSLSPGERATLSCRASQSVSSYLAWYQQKPGQAPRLLIY |
| Region | DASNRATGIPARFSGSGSGTDFTLTISSLEPEDFAVYYCQQSSNWPRTF |
| | GQGTKVEIK |
| | (SEQ ID NO: 247) |
| Light | EIVLTQSPATLSLSPGERATLSCRASQSVSSYLAWYQQKPGQAPRLLIY |
| Chain | DASNRATGIPARFSGSGSGTDFTLTISSLEPEDFAVYYCQQSSNWPRTF |
| | GQGTKVEIKRTVAAPSVFIFPPSDEQLKSGTASVVCLLNNFYPREAKVQ |
| | WKVDNALQSGNSQESVTEQDSKDSTYSLSSTLTLSKADYEKHKVYAC |
| | EVTHQGLSSPVTKSFNRGEC |
| | (SEQ ID NO: 248) |
| Nivolumab Heavy Chain | |
| CDR1 | NSGMH |
| | (SEQ ID NO: 249) |
| CDR2 | VIWYDGSKRYYADSVKG |
| | (SEQ ID NO: 250) |
| CDR3 | NDDY |
| | (SEQ ID NO: 251) |
| Variable | QVQLVESGGGVVQPGRSLRLDCKASGITFSNSGMHWVRQAPGKGLE |
| Region | WVAVIWYDGSKRYYADSVKGRFTISRDNSKNTLFLQMNSLRAEDTA |
| | VYYCATNDDYWGQGTLVTVSS |
| | (SEQ ID NO: 252) |
| Heavy | QVQL VESGGGVVQPGRSLRLDCKASGITFSNSGMHWVRQAPGKGLE |
| Chain | WVAVIWYDGSKRYYADSVKGRFTISRDNSKNTLFLQMNSLRAEDTAV |
| | YYCATNDDYWGQGTLVTVSSASTKGPSVFPLAPCSRSTSESTAALGC |
| | LVKDYFPEPVTVSWNSGALTSGVHTFPAVLQSSGLYSLSSVVTVPSS |
| | SLGTKTYTCNVDHKPSNTKVDKRVESKYGPPCPPCPAPEFLGGPSVF |
| | LFPPKPKDTLMISRTPEVTCVVVDVSQEDPEVQFNWYVDGVEVHNAK |
| | TKPREEQFNSTYRVVSVLTVLHQDWLNGKEYKCKVSNKGLPSSIEKT |
| | ISKAKGQPREPQVYTLPPSQEEMTKNQVSLTCLVKGFYPSDIAVEWE |
| | SNGQPENNYKTTPPVLDSDGSFFLYSRLTVDKSRWQEGNVFSCSVMH |
| | EALHNHYTQKSLSLSLGK |
| | (SEQ ID NO: 253) |

The anti-ILT3 antigen binding proteins or antigen binding fragments herein may be used alone or in combination with other therapies. For example, the combination therapy may include a composition comprising an anti-ILT3 antigen binding protein, antibody or antigen binding fragment co-formulated with, and/or co-administered with, one or more additional therapeutic agents, e.g., one or more anti-cancer agents, cytotoxic or cytostatic agents, hormone treatment, vaccines, and/or other immunotherapies. In other embodiments, the anti-ILT3 antigen binding protein, antibody or antigen binding fragment is administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the various monotherapies.

By “in combination with,” it is not intended to imply that the therapy or the therapeutic agents must be administered at the same time and/or formulated for delivery together, although these methods of delivery are within the scope described herein. The anti-ILT3 antigen binding protein, antibody or antigen binding fragment may be administered concurrently with, prior to, or subsequent to, one or more other additional therapies or therapeutic agents. The anti-ILT3 antigen binding protein, antibody or antigen binding fragment and the other agent or therapeutic protocol may be administered in any order. In general, each agent will be administered at a dose and/or on a time schedule determined for that agent. In will further be appreciated that the additional therapeutic agent utilized in this combination may be administered together in a single composition or administered separately in different compositions. In general, it is expected that additional therapeutic agents utilized in combination be utilized at levels that do not exceed the levels at which they are utilized individually. In some embodiments, the levels utilized in combination will be lower than those utilized individually.

In certain embodiments, an anti-ILT3 antigen binding protein or antigen binding fragment described herein is administered in combination with one or more check point inhibitors or antagonists of programmed death receptor 1 (PD-1) or its ligand PD-L1 and PD-L2. The inhibitor or antagonist may be an antigen binding protein, an antibody, an antigen binding fragment, an immunoadhesin, a fusion protein, or oligopeptide. In some embodiments, the anti-PD-1 antibody is chosen from nivolumab (OPDIVO®, Bristol Myers Squibb, New York, New York), pembrolizumab (KEYTRUDA®, Merck Sharp & Dohme Corp, Kenilworth, NJ USA), cetiplimab (Regeneron, Tarrytown, NY) or pidilizumab (CT-011). In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 inhibitor is AMP-224. In some embodiments, the PD-L1 inhibitor is anti-PD-L1 antibody such durvalumab (IMFINZI®, Astrazeneca, Wilmingon, DE), atezolizumab (TECENTRIQ®, Roche, Zurich, CH), or avelumab (BAVENCIO®, EMD Serono, Billerica, MA). In some embodiments, the anti-PD-L1 binding antagonist is chosen from YW243.55.S70, MPDL3280A, MEDI-4736, MSB-0010718C, or MDX-1105.

MDX-1105, also known as BMS-936559, is an anti-PD-L1 antibody described in WO2007/005874. Antibody YW243.55.S70 is an anti-PD-L1 described in WO 2010/077634 (heavy and light chain variable region sequences shown in SEQ ID NOs. 20 and 21, respectively).

Nivolumab, also known as OPDIVO®, MDX-1106-04, ONO-4538, or BMS-936558, is a fully human IgG4 anti-PD-1 antibody described in WO2006/121168 and U.S. Pat. No. 8,008,449.

Pembrolizumab, also known as KEYTRUDA®, lambrolizumab, MK-3475 or SCH-900475, is a humanized anti-PD-1 antibody described in U.S. Pat. No. 8,354,509 and WO2009/114335 and disclosed, e.g., in Hamid, et al., New England J. Med. 369 (2): 134-144 (2013). The heavy and light chains for pembrolizumab are shown by the amino acid sequences set forth in SEQ ID Nos: 225 and 226, respectively.

Pidilizumab, also known as CT-011 (Cure Tech) is a humanized IgG1 monoclonal antibody that binds to PD-1. Pidilizumab and other humanized anti-PD-1 monoclonal antibodies are disclosed in WO2009/101611. Other anti-PD-1 antibodies include AMP 514 (Amplimmune), among others, e.g., anti-PD-1 antibodies disclosed in U.S. Pat. No. 8,609,089; U.S Publication No. 2010028330; and U.S Publication No. 20120114649

AMP-514 (MEDI0680; MedImmune LLC, Gaithersburg, MD) is a monoclonal antibody that binds PD-1.

PDR001 (spartalizumab; Novartis) is a monoclonal antibody that binds PD-1 and is disclosed in U.S. Pat. No. 9,683,048.

BGB-A317 (tislelizumab; Beigene) is a monoclonal antibody that binds PD-1 and is disclosed in U.S. Pat. No. 8,735,553.

MDPL3280A (Genentech/Roche) is a human Fc optimized IgG1 monoclonal antibody that binds to PD-L1. MDPL3280A and other human monoclonal antibodies to PD-L1 are disclosed in U.S. Pat. No. 7,943,743 and U.S Publication No. 20120039906.

MGA012 (MacroGenics, Rockville, MD) a monoclonal antibody that binds PD-1.

AMP-224 (B7-DCIg; Amplimmune; e.g., disclosed in WO2010/027827 and WO2011/066342), is a PD-L2 Fc fusion soluble receptor that blocks the interaction between PD-1 and B7-H1.

Other anti-PD-L1 binding agents include YW243.55.S70 (heavy and light chain variable regions are shown in SEQ ID NOs 20 and 21 in WO2010/077634) and MDX-1105 (also referred to as BMS-936559). It and other anti-PD-L1 binding agents are disclosed in WO2007/005874).

### Dosing and Administration

Provided herein are dosing regimens and routes of administration for treating cancer and in specific embodiments, mTNBC, GBM, mPDAC, mSTS, or mNSCLC using an anti-ILT3 antigen binding protein or antigen binding fragment (e.g., any of the mAbs in Table 8), or a combination of an anti-ILT3 antigen binding protein or antigen binding fragment (e.g., any of the mAbs in Table 8) and an anti-PD-1 antigen binding protein or antigen binding fragment (e.g., pembrolizumab).

The anti-ILT3 antigen binding protein or antigen binding fragment and the anti-PD1 antigen binding protein or antigen binding fragment disclosed herein may be administered by continuous infusion, or by doses administered, e.g., daily, 1-7 times per week, weekly, biweekly, tri-weekly, every four weeks, every five weeks, every 6 weeks, monthly, bimonthly, quarterly, semiannually, annually, etc., either concurrently or consecutively. Doses may be administered, e.g., intravenously, subcutaneously, topically, orally, nasally, rectally, intramuscular, intracerebrally, intraspinally, or by inhalation. In certain embodiments, the doses are administered intravenously. In certain embodiments, the doses are administered subcutaneously. A total dose for a treatment interval is generally at least 0.05 μg/kg body weight, more generally at least 0.2 μg/kg, 0.5 μg/kg, 1 μg/kg, 10 μg/kg, 100 μg/kg, 0.25 mg/kg, 1.0 mg/kg, 2.0 mg/kg, 5.0 mg/ml, 10 mg/kg, 25 mg/kg, 50 mg/kg or more. Doses may also be provided to achieve a pre-determined target concentration of the antigen binding protein (e.g., anti-ILT3 antibody or anti-PD1 antibody) or antigen binding fragment in the subject's serum, such as 0.1, 0.3, 1, 3, 10, 30, 100, 300 μg/mL or more. In some embodiments, the anti-ILT3 antigen binding protein or antigen binding fragment is administered intravenously, on a weekly, biweekly, triweekly, every 3 weeks, every 4 weeks, every 5 weeks, every 6 weeks, monthly, bimonthly, or quarterly basis at 10, 20, 50, 80, 100, 200, 300, 400, 500, 1000 or 2500 mg/subject. In some embodiments, the anti-PD-1 antigen binding protein or antigen binding fragment is administered subcutaneously or intravenously, on a weekly, biweekly, triweekly, every 3 weeks, every 4 weeks, every 5 weeks, every 6 weeks, monthly, bimonthly, or quarterly basis at 10, 20, 50, 80, 100, 200, 300, 400, 500, 1000 or 2500 mg/subject.

In some embodiments, the anti-ILT3 antigen binding protein or antigen binding fragment is administered intravenously, on a weekly, biweekly, triweekly, every 4 weeks, every 5 weeks, every 6 weeks, monthly, bimonthly, or quarterly basis at 10, 20, 50, 80, 100, 200, 500, 1000 or 2500 mg/subject. In some specific methods, the dose of the anti-ILT3 antigen binding protein or antigen binding fragment is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, from about 2.0 mg/kg to about 4.0 mg/kg. In some specific methods, the dose of an anti-ILT3 antigen binding protein or antigen binding fragment may be between about 0.2 mg and about 2 mg. In some specific methods, the dose of an anti-ILT3 antigen binding protein or antigen binding fragment may be between 0.2 mg and 2 mg. In some specific methods, the dose of an anti-ILT3 antigen binding protein or antigen binding fragment may be between about 0.2 mg and about 2250 mg. In some specific methods, the dose of an anti-ILT3 antigen binding protein or antigen binding fragment may be between 0.2 mg and 2250 mg. In some specific methods, the dose of an anti-ILT3 antigen binding protein or antigen binding fragment may be between about 7.5 mg and about 2250 mg. In some specific methods, the dose of an anti-ILT3 antigen binding protein or antigen binding fragment may be between 7.5 mg and 2250 mg. In some specific methods, the dose of an anti-ILT3 antigen binding protein or antigen binding fragment may be about 0.2 mg, about 0.7 mg, or about 2 mg. In some specific methods, the dose of an anti-ILT3 antigen binding protein or antigen binding fragment may be about 7.5 mg, about 25 mg, about 75 mg, about 225 mg, about 750 mg, or about 2250 mg. In some specific methods, the dose of an anti-ILT3 antigen binding protein or antigen binding fragment may be 0.2 mg, 0.7 mg, or 2 mg. In some specific methods, the dose of an anti-ILT3 antigen binding protein or antigen binding fragment may be 7.5 mg, 25 mg, 75 mg, 225 mg, 750 mg, or 2250 mg. In some specific methods, the dose of an anti-ILT3 antigen binding protein or antigen binding fragment may be about 750 mg. In some specific methods, the dose of an anti-ILT3 antigen binding protein or antigen binding fragment may be 750 mg.

In some embodiments, the anti-PD-1 antigen binding protein or antigen binding fragment is administered intravenously, concurrently or consecutively with the anti-ILT3 antigen binding protein, on a weekly, biweekly, triweekly, every 4 weeks, every 5 weeks, every 6 weeks, monthly, bimonthly, or quarterly basis at 10, 20, 50, 80, 100, 200, 500, 1000 or 2500 mg/subject. In some specific methods, the dose of the anti-PD-1 monoclonal antibody or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, from about 2.0 mg/kg to about 4.0 mg/kg. In some specific methods, the dose of the anti-PD-1 monoclonal antibody or antigen binding fragment thereof is from about 10 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, from about 200 mg to about 240 mg. In some embodiments, the dose of the anti-PD-1 antigen binding protein or antigen binding fragment thereof is about 1 mg, about 5 mg, about 10 mg, about 15 mg, about 20 mg, about 30 mg, about 40 mg, about 45 mg, about 50 mg, about 75 mg, about 100 mg, about 125 mg, about 150 mg, about 175 mg, about 200 mg, about 225 mg, about 240 mg, about 250 mg, about 300 mg, about 400 mg, or about 500 mg. In some embodiments, the dose of the anti-PD-1 monoclonal antibody or antigen binding fragment thereof is 50 mg, 75 mg, 100 mg, 125 mg, 150 mg, 175 mg, 200 mg, 225 mg, 240 mg, 250 mg, 300 mg, 400 mg, or 500 mg.

### Methods and Uses of the Invention

The present invention provides a method for treating a cancer in a subject comprising administering to the subject an effective amount of an anti-ILT3 antigen binding protein or antigen binding fragment. In other embodiments, the present invention provides a method for treating a cancer in a subject comprising administering to the subject an effective amount of an anti-ILT3 antigen binding protein or antigen binding fragment and an anti-PD-1 or anti-PD-L1 and/or PD-L2 antigen binding protein or antigen binding fragment disclosed or claimed herein sufficient to treat the cancer in the subject.

The present invention provides a method for treatment of cancer in a subject comprising administering to the subject an effective amount of an anti-ILT3 antigen binding protein or antigen binding fragment. The present invention further provides a method for treatment of a cancer in a subject comprising administering to the subject concurrently or consecutively an anti-ILT3 antigen binding protein or antigen binding fragment disclosed herein in combination with one or more inhibitors or antagonists of PD-1, PD-L1 and/or PD-L2. In one embodiment, the antagonist of PD-1 is an antibody or antigen binding fragment that binds to human PD-1 and blocks the binding of PD1 to human PD-L1 and PD-L2. In one embodiment, the antagonist of PD-L1 or PD-L2 is an antibody or antigen binding fragment that binds to human PD-L1 or PD-L2 and blocks the binding of human PD-L1 or PD-L2 PD1.

In a further embodiment, the anti PD1 antagonist is an anti-PD-1 antibody. In one embodiment, the anti-PD-1 antibody is nivolumab, pembrolizumab, cemiplimab, pidilizumab, AMP-514, PD001, BGB-A317, MDPL3280A, or MGA012 and the PD-L1 inhibitor is durvalumab, atezolizumab, avelumab, YW243.55.S70, MPDL3280A, MEDI-4736, MSB-0010718C, or MDX-1105.

In a further embodiment, the cancer being treated in the methods of treatment disclosed herein is pancreatic cancer, melanomas, breast cancer, lung cancer, head and neck cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, or chondrosarcoma.

In some embodiments, the cancer is metastatic triple negative breast cancer (mTNBC), recurrent non-operable glioblastoma (GBM), metastatic pancreatic ductal adenocarcinoma (mPDAC), metastatic soft tissue sarcoma (mSTS), or metastatic non-squamous non-small cell lung carcinoma (mNSCLC).

### General Methods

Standard methods in molecular biology are described Sambrook, Fritsch and Maniatis (1982 & 1989 2nd Edition, 2001 3rd Edition) Molecular Cloning, A Laboratory Manual, Cold Spring Harbor Laboratory Press, Cold Spring Harbor, NY; Sambrook and Russell (2001) Molecular Cloning, 3rd ed., Cold Spring Harbor Laboratory Press, Cold Spring Harbor, NY; Wu (1993) Recombinant DNA, Vol. 217, Academic Press, San Diego, CA). Standard methods also appear in Ausubel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Shepherd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies or antigen binding fragments can be conjugated, e.g., to small drug molecules, enzymes, liposomes, polyethylene glycol (PEG). Antibodies are useful for therapeutic, diagnostic, kit, or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY). Software packages and databases for determining, e.g., antigenic fragments, leader sequences, protein folding, functional domains, glycosylation sites, and sequence alignments, are available (see, e.g., GenBank, VECTOR NTI Suite (Informax, Inc, Bethesda, MD); GCG Wisconsin Package (Accelrys, Inc., San Diego, CA); DECYPHER (TimeLogic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### EXAMPLES

The following examples using mAb number 46 as a representative anti-ILT3 antibody are meant to be illustrative and should not be construed as further limiting. The contents of the figures and all references, patents, and published patent applications cited throughout this application are expressly incorporated herein by reference.

### Example 1: Dose Escalation and Cohort Expansion Study to Evaluate the Safety, Tolerability, Pharmacokinetics and Pharmacodynamics of an Anti-ILT3 Antibody

### Arm 1: Anti-ILT3 Antibody Monotherapy Dose Escalation Accelerated Titration Design (ATD)

Dose escalation is initiated by evaluating anti-ILT3 antibody monotherapy using an ATD to minimize the number of participants treated at potentially subtherapeutic doses of anti-ILT3 antibody. In the ATD, 1 to 3 participants per cohort will be treated at increasing DL of anti-ILT3 antibody with up to one-half log unit dose increases between successive levels (see FIG. 1A, FIG. 1B).

Transition from the ATD to the mTPI (modified toxicity probability interval) design will be triggered by the occurrence of either or both of the following events: 1) ≥Grade 2 toxicity as assessed by the investigator to be related, probably related, or possibly related to the drug at any DL during the DLT period (Cycle 1); or 2) the highest DL cohort in ATD has completed the DLT evaluation period and anti-ILT3 antibody at that DL has been determined to be safe and well tolerated in this cohort, and if data are available, levels ≥75% ILT3 receptor occupancy in peripheral blood mononuclear cells are demonstrated at any ATD DL.

Intra-participant dose escalation will be allowed for participants in the ATD. Participants may undergo more than 1 dose escalation. For this to occur, the participant must have completed at least 1 cycle of anti-ILT3 antibody (i.e., DLT period for that DL) without a ≥Grade 2 study drug-related toxicity and the safety of the higher DL must have already been evaluated in at least 1 participant (i.e., the participant at the higher DL must have completed the DLT period). For participants undergoing more than 1 dose escalation, their contribution to DLT will be counted only once at the initial cohort to which they were enrolled, and biopsy sample collection will not be repeated.

Modified Toxicity Probability Interval Design (mTPI)

After the ATD phase is completed, dose escalation of anti-ILT3 antibody monotherapy will continue using an mTPI design ((Ji Y, Wang S-J. Modified toxicity probability interval design: a safer and more reliable method than the 3+3 design for practical phase I trials. J Clin Oncol 2013; 31: 1-12.) to identify the MTD and/or MAD, targeting a DLT rate of 30%. The starting dose of anti-ILT3 antibody for mTPI will be based on the available safety, PK, and pharmacodynamics results from the ATD. Lower and/or higher doses and additional cohorts of anti-ILT3 antibody may be explored depending on the combined safety, PK, and pharmacodynamics data available at each DL.

During mTPI dose escalation, 3 to 6 participants will be initially enrolled to receive anti-ILT3 antibody monotherapy. Treatment allocation will be accomplished by non-random assignment. Enrollment in Arm 1 (anti-ILT3 antibody as monotherapy) at the next DL will begin once all participants in the current DL complete DLT evaluation of 21 days and a dose escalation decision has been made. At least 24 hours must pass between when the first and second participants receive study treatment in Cycle 1 of each new DL. Each subsequent DL will proceed similarly. Intra-participant dose escalation is not permitted in the mTPI phase of Arm 1.

Participants who discontinue anti-ILT3 antibody at any DL in Arm 1 due to progressive disease may, at the investigator's discretion and after consultation with and approval by the Sponsor, be eligible to receive combination treatment with pembrolizumab (please see sub-heading entitled “Transition to Combination Therapy” below for more details).

Collection of tumor biopsies (at screening and C3D1) is mandatory for Arm 1, unless a biopsy is deemed medically unsafe by the investigator.

### Arm 2: Combination Therapy Dose Escalation Modified Toxicity Probability Interval Design

The starting dose for Arm 2 will be 2 DL below the current dose being tested in mTPI in Arm 1. Therefore, enrollment in Arm 2 will begin once all Arm 1 participants complete the DLT evaluation period of DL2 in monotherapy and anti-ILT3 antibody has been demonstrated to be safe and tolerable in this cohort and a dose escalation decision has been made. The starting dose in Arm 2 may be adjusted based on the available safety, PK, and pharmacodynamics results from Arm 1. Dose escalation will proceed using the mTPI design to determine the MTD and/or MAD of anti-ILT3 antibody in combination with pembrolizumab; Arm 2 will always be 2 DL behind Arm 1 until dose escalation in Arm 1 is completed. Based on the emerging safety and/or efficacy signals, intermediate dose levels may also be explored. The final number of participants enrolled in Part 1 will depend on the empirical safety observations (ie, DLT), and what dose is ultimately identified as the MTD/MAD using the mTPI design. For Arm 2, collection of tumor biopsies (at Screening and C3D1) is optional but strongly encouraged, especially at C3D1 visit, unless a biopsy is deemed medically unsafe by the investigator.

The dose of pembrolizumab in Part 1, Arm 2 will remain constant at 200 mg Q3W.

Dose finding and confirmation in Arm 2 will end after a maximum of 14 participants have been treated at any of the selected doses (which may include intermediate doses). Dose escalation will stop if the mTPI table indicates “S” for staying at current dose. Otherwise, up to 14 new participants may be enrolled at a lower dose if “D” or “DU” is indicated or a higher dose of “E” is indicated. The PAVA ((Ji Y, Wang S-J. Modified toxicity probability interval design: a safer and more reliable method than the 3+3 design for practical phase I trials. J Clin Oncol 2013; 31:1-12.) will be used to estimate the DLT rates across doses in each treatment arm under the assumption of monotonicity between DLT rates and DLs. The dose with an estimated DLT rate closest to 30% may be treated as an MTD/MAD. The totality of the data will be considered before deciding on the RP2D dose(s) to carry forward. The MTD/MAD of anti-ILT3 antibody in Arm 2 will not exceed, but may equal, the MTD/MAD in the anti-ILT3 antibody Arm 1. Intra-participant dose escalation is not permitted in the mTPI phase of Arm 2.

Preliminary efficacy will be evaluated using ORR, PFS, and OS as exploratory endpoints. ORR and PFS will be assessed by the investigator based on RECIST 1.1 and iRECIST.

Accumulating data will be examined on a continuous basis to allow for dose finding decisions based on ATD and mTPI and to enable future study planning at the Sponsor's discretion.

### Transition to Combination Therapy

Participants who demonstrate radiographically confirmed progressive disease in Part 1 Arm 1, will be eligible to receive combination therapy (ie, ‘cross-over’ into Arm 2), after consultation with and approval by the Sponsor.

A participant may not cross over from Arm 1 (monotherapy) into Arm 2 (combination therapy with pembrolizumab) until that participant has completed the DLT evaluation period (ie, 21 days) in Arm 1. Participants who are eligible for crossover from Arm 1 to Arm 2 will enter Arm 2 at Screening and will be allocated to the highest open combination DL (see Section 6.6.3). These participants will continue to undergo their scheduled activities with the addition of pembrolizumab PK and ADA assessments, as appropriate. Participants may receive the highest dose of anti-ILT3 antibody that has already demonstrated safety and tolerability in combination with pembrolizumab (DLT evaluation period completed for that combination dose).

Participants who are eligible to receive combination treatment due to radiographically confirmed progressive disease while in Arm 1, will not be included in the mTPI dose escalation determination for Arm 2, as that specific DL cohort must have already demonstrated safety and tolerability. However, their data may be included retrospectively in determination of the RP2D for the combination treatment. These participants' safety and efficacy data will be analyzed separately from that of the participants enrolled in Arm 2.

Once they discontinue from any part of the study, participants will be treated at the discretion of the physician.

Participants who cross over to combination treatment will be eligible to receive a maximum of 35 cycles of combination treatment irrespective of the number of cycles of anti-ILT3 antibody received in monotherapy.

Participants who discontinued monotherapy in Arm 1 due to a DLT are not eligible for cross-over to Arm 2.

### Cohort Expansion

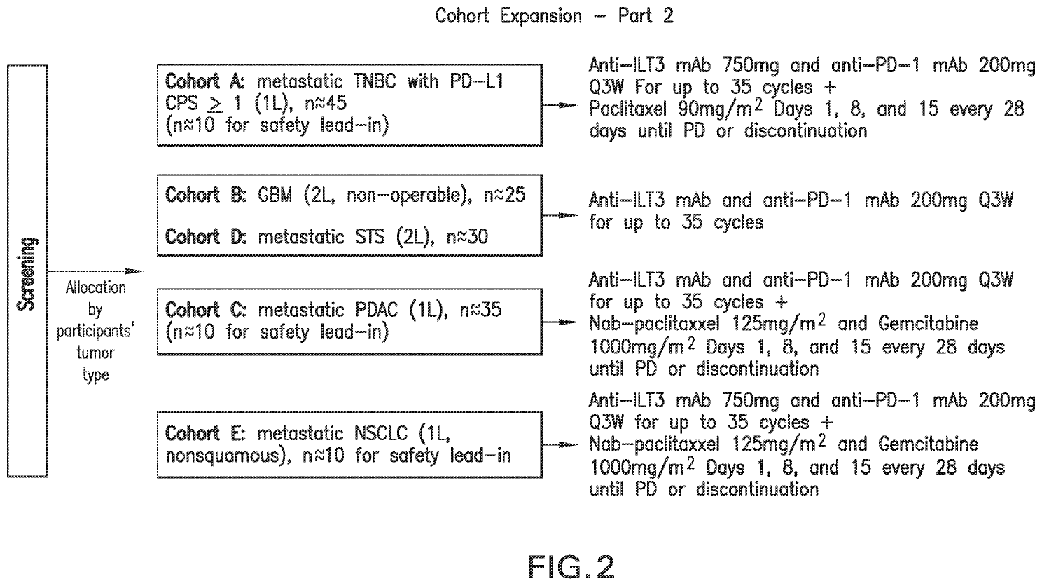

This study includes 5 tumor-specific cohorts to evaluate anti-ILT3 antibody plus pembrolizumab 200 mg Q3W with chemotherapy (Cohort A, C, and E) or without chemotherapy (Cohort B and D) as shown in FIG. 2.

Cohort A will enroll approximately 45 treatment-naïve participants with PD-L1 CPS ≥1 metastatic TNBC to evaluate the safety and preliminary efficacy of anti-ILT3 antibody in combination with pembrolizumab and paclitaxel. Cohort A includes a safety lead-in with approximately 10 participants to demonstrate a tolerable safety profile of the combination before continuing with the full enrollment.

Cohort B will enroll approximately 25 participants with 2L non-operable GBM to evaluate the safety and preliminary efficacy of anti-ILT3 antibody in combination with pembrolizumab.

Cohort C will enroll approximately 35 participants with 1L metastatic PDAC to evaluate the safety and preliminary efficacy of anti-ILT3 antibody in combination with pembrolizumab, nab-paclitaxel, and gemcitabine. Cohort C includes a safety lead-in with approximately 10 participants to demonstrate a tolerable safety profile of the combination before continuing with the full enrollment.

Cohort D will enroll approximately 30 participants with 2L metastatic STS to evaluate the safety and preliminary efficacy of anti-ILT3 antibody in combination with pembrolizumab.

Cohort E will enroll approximately 10 treatment-naïve participants with metastatic non-squamous NSCLC as a safety lead-in to demonstrate a tolerable safety profile of anti-ILT3 antibody in combination with pembrolizumab, carboplatin, and pemetrexed.

Participants will be permitted to continue study treatment beyond progression following Sponsor consultation if investigator-assessed clinical stability is observed and the participant is tolerating study treatment.

An interim analysis (IA) may be conducted after the first 15 participants (Cohorts B, C, and D) or 20 participants (Cohort A) have their second post-baseline imaging assessment. If 8 or fewer responses (Cohort A), 3 or fewer responses (Cohort C), or 1 or fewer responses (Cohorts B and D) are observed, enrollment in the cohort may be stopped early.

### Safety Lead-In for Chemotherapy Combinations

For the first approximately 10 participants in Cohorts A and C and all participants in Cohort E, the mTPI table (Table 10) with a target dose limiting toxicity (DLT) rate of 30% will be applied to evaluate the safety and tolerability of the intended dose of chemotherapy in the triplet or quadruplet combinations for each cohort separately. Three to 6 DLT-evaluable participants will initially be enrolled and evaluated for DLTs from first dose of study intervention. Up to 8 participants may be enrolled initially to achieve the desired sample size of 6 DLT-evaluable participants. If the decision based on the mTPI table (see Table 10 herein) is to stay or escalate, the cohort will be expanded to enroll additional participants to have a total of 10 DLT-evaluable participants. If a dose de-escalation decision is made, enrollment in the cohort may be delayed to further evaluate the safety data of the combination and to determine if the cohort should be expanded. If data from the safety lead-in indicate that a combination has acceptable safety and tolerability, enrollment in the cohort will continue. If data from the safety lead-in are not acceptable, enrollment in the cohort will stop.

The DLT evaluation period is 28 days for Cohorts A and C and 21 days for Cohort E.

### Follow-Up

Participants who discontinue study treatment for reasons other than confirmed progressive disease will have post-treatment follow-up for safety and disease status (including imaging) until progressive disease, initiating a new anticancer therapy, pregnancy, withdrawal of consent for study participation, death, or becoming lost to follow-up.

Participants who experience confirmed disease progression or start a new anticancer therapy, will move into Safety and Survival Follow-up Phases.

### Objective Response Rate (ORR)

This study will use ORR based on RECIST 1.1 criteria (RANO for Cohort B) as assessed by the investigator as a secondary endpoint.

Tumor response in participants will be assessed using the RECIST 1.1 and the iRECIST criteria by investigator review. A central imaging vendor will be used to collect, clean, and hold tumor imaging. Images will be collected for possible future analysis by BICR.

### Response Rate Assessed by RECIST 1.1

RECIST 1.1 will be used by the investigator when assessing images for efficacy measures and by the local site when determining eligibility. Although traditional RECIST 1.1 references a maximum of 5 target lesions in total and 2 per organ, this protocol has implemented a modification to RECIST 1.1 to allow a maximum of 10 target lesions in total and 5 per organ.

Response Rate Assessed by Modified Response Evaluation Criteria in Solid Tumors 1.1 for Immune-Based Therapeutics (iRECIST)

RECIST 1.1 has been adapted to account for the unique tumor response characteristics seen following treatment with pembrolizumab. Immunotherapeutic agents such as anti-ILT3 antibody and pembrolizumab may produce antitumor effects by potentiating endogenous cancer-specific immune responses. The response patterns seen with such an approach may extend beyond the typical time course of responses seen with cytotoxic agents, and patients treated with immunotherapeutic agents may manifest a clinical response after an initial increase in tumor burden or even the appearance of new lesions. Thus, standard RECIST 1.1 may not provide an accurate response assessment of immunotherapeutic agents such as anti-ILT3 antibody and pembrolizumab. Based on an analysis of participants with melanoma enrolled in KEYNOTE-001 (KN001), 7% of evaluable participants experienced delayed or early tumor pseudo-progression. Of note, participants who had progressive disease by RECIST 1.1 but not by the immune-related response criteria (Wolchok J D et al. Guidelines for the evaluation of immune therapy activity in solid tumors: immune-related response criteria. Clin Cancer Res 2009; 15(23):7412-20) had longer overall survival than participants with progressive disease by both criteria (Hodi F S et al. Patterns of response in patients with advanced melanoma treated with Pembrolizumab

(MK-3475) and evaluation of immune related response criteria (irRC). J Immunother Cancer. 2014; 2(Suppl 3): P103). Additionally, the data suggest that RECIST 1.1 may underestimate the benefit of pembrolizumab in approximately 15% of participants. These findings support the need to apply a modification to RECIST 1.1 that takes into account the unique patterns of atypical responses in immunotherapy and enables treatment beyond initial radiographic progression, if the participant is clinically stable.

Modified RECIST 1.1 for immune-based therapeutics (iRECIST) assessment has been developed and published by the RECIST Working Group, with input from leading experts from industry and academia, along with participation from the US FDA and the European Medicines Agency (Seymour L, et al. iRECIST: guidelines for response criteria for use in trials testing immunotherapeutics. Lancet Oncol. 2017 March; 18(3):e143-52). The unidimensional measurement of target lesions, qualitative assessment of non-target lesions, and response categories are identical to RECIST 1.1, until progression is seen by RECIST 1.1. However, if a participant is clinically stable, additional imaging may be performed to confirm radiographic progression. iRECIST will be used by investigators to assess tumor response and progression and make treatment decisions as well as for exploratory efficacy analyses where specified.

### Response Assessment in Neuro-Oncology (RANO)

RANO criteria have been the preferred criteria for assessing responses in GBM trials since their publication in 2010 (Wen P Y, Macdonald D R, Reardon D A, et al. Updated response assessment criteria for high-grade gliomas: response assessment in neuro-oncology working group. J Clin Oncol 2010; 28(11): 1963-72) and incorporate measurements of tumor size as demonstrated in contrast-enhanced MRI with qualitative assessment of both enhancing and nonenhancing disease, and information on steroid dosing and participant functional performance status. Response assessments will be performed by investigators and by BICR.

RANO also makes provisions for the pseudoprogression frequently seen following radiotherapy. The AVAglio study (Gilbert M R et al. A randomized trial of bevacizumab for newly diagnosed glioblastoma. N Engl J Med. 2014 Feb. 20; 370(8):699-708.) (BO21990, NCT00943826) modified the Macdonald criteria by using T2/FLAIR imaging, clinical assessment, and the qualitative review of all non-index lesions to correct for non contrast-enhancing lesions, residual disease, difficult to measure lesions, and pseudoprogression. The RANO Working Group further refined the measurements by relaxing criteria around clinical progression and in the timing, criteria, and confirmation of scans to detect pseudoprogression (Chinot O L et al. Response assessment criteria for glioblastoma: practical adaptation and implementation in clinical trials of antiangiogenic therapy. Curr Neurol Neurosci Rep. 2013 May; 13(5):347).

RTOG and ACRIN (RTOG0625/ACRIN6677) evaluated the predictive ability of RANO in 107 patients with recurrent GBM treated with bevacizumab, irinotecan, or temozolomide (Boxerman J L et al. Early post-bevacizumab progression on contrast enhanced MRI as a prognostic marker for overall survival in recurrent glioblastoma: results from the ACRIN 6677/RTOG 0625 Central Reader Study. Neuro Oncol. 2013 July; 15(7):945-54). The study concluded that progression observed at 8 and 16 weeks of bevacizumab treatment on 2D-T1 and 3D-T1 imaging, had highly significant prognostic value for OS. However, progression detected by FLAIR alone did not correlate with OS and added minimal additional benefit to other imaging technologies.

### Progression-Free Survival

This study will use PFS as assessed by the investigator according to RECIST 1.1 (RANO for Cohort B) and iRECIST criteria (see above), modified to follow a maximum of 10 target lesions and a maximum of 5 target lesions per organ, as an exploratory endpoint. For late stage studies, PFS is an acceptable measure of clinical benefit and will be used in this FIH study to provide a preliminary measure of efficacy of anti-ILT3 antibody in combination with pembrolizumab, and in combination with pembrolizumab and chemotherapy in advanced solid tumors.

The PFS rate at 6 months, 12 months, 18 months, and 24 months for Cohorts A, B, C, and D, respectively, will also be evaluated.

### Overall Survival (OS)

Overall survival has been recognized as the gold standard for the demonstration of superiority of a new antineoplastic therapy in randomized clinical studies. In this study, OS will be measured as an exploratory endpoint. The OS endpoint may be potentially confounded by the small sample sizes and absence of a control group for comparison, limiting its utility as a secondary endpoint. This study will enroll participants with different types of advanced solid tumors and this heterogeneity combined with the variability in salvage procedures will impact the utility of the OS exploratory endpoint.

The OS rate at 6 months, 12 months, 18 months, and 24 months for Cohorts A, B, C, and D, respectively, will also be evaluated.

### Safety Endpoints

An objective of this study is to characterize the safety and tolerability of anti-ILT3 antibody as combination therapy with pembrolizumab, and as combination therapy with pembrolizumab and chemotherapy in participants with advanced/metastatic solid tumors.

The primary safety analysis will be based on participants who experience toxicities as defined by NCI CTCAE, version 4.0 criteria. Safety will be assessed by quantifying the toxicities and grades of toxicities experienced by participants who have received anti-ILT3 antibody as monotherapy and in combination with pembrolizumab with and without chemotherapy.

For AEs, attribution to drug, time-of-onset, duration of the event, its resolution, and any concomitant medications administered will be recorded. Adverse events that will be analyzed include, but are not limited to, all AEs, SAEs, fatal AEs, and laboratory changes.

### Rationale for Participant Populations

Participant populations for the cohort expansion were selected based on an analysis of human tumor expression arrays within the Moffitt and The Cancer Genome Atlas (TCGA) databases. The inventors analyzed levels of ILT3 expression, and T cell-inflamed gene expression profile scores, (GEP scores; see Cristecu et al. Science. 2018 Oct. 12; 362(6411): eaar3593). The GEP expression profile includes 18 inflammatory genes related to antigen presentation, chemokine expression, cytolytic activity, and adaptive immune resistance, including CCL5, CD27, CD274 (PD-L1), CD276 (B7-H3), CD8A, CMKLR1, CXCL9, CXCR6, HLA-DQA1, HLA-DRB1, HLA-E, IDO1, LAG3, NKG7, PDCDILG2 (PDL2), PSMB10, STAT1, and TIGIT. High GEP scores indicate a T cell-inflamed tumor microenvironment.

Tumor types with high levels of ILT3 expression, a correlation between high ILT3 expression and GEP scores, an MDSC-enriched tumor microenvironment, as well as unmet medical need, were identified. Surprisingly, the inventors made several key findings around particular cancer types through their analysis, as compared to other cancer types. The inventors found that a large percentage of GBM tumors showed high levels of ILT3 expression, and a low GEP score. Tumor myeloid cells in GBM account for 30-50% of tumor mass, and the majority of those cells are monocytic MDSCs. Immune suppression in GBM appears to be from macrophages, not microglia. NSCLC and TNBC showed a large percentage of tumors with a high GEP score and high levels of ILT3 expression; TNBC has a limited response to pembrolizumab monotherapy. PDAC and STS shows a moderate percentage of tumors with a high GEP score and high levels of ILT3 expression. The tumor environment for PDAC includes immune cells.

Based on the key findings of the inventors noted above, it is hypothesized that the anti-ILT3 antigen binding protein- or antigen binding fragment-mediated inhibition of ILT3 in tumors with these attributes will reverse the tolerance or immune suppression seen in the tumor microenvironment and may show antitumor activity when used as a monotherapy or in combination with an anti-PD-1 antigen binding protein or antigen binding fragment (e.g., pembrolizumab) or an anti-PD-1 antigen binding protein or antigen binding fragment (e.g., pembrolizumab) and a standard of care chemotherapy in an additive or synergistic fashion. This strategy using anti-ILT3 antigen binding proteins or antigen binding fragments alone or in combination with an anti-PD-1 antigen binding protein or antigen binding fragment and in particular in the manner described herein has not be exploited as a therapeutic strategy for these difficult to treat cancers.

Based on the above criteria, Part 2 will enroll participants with treatment-naïve metastatic TNBC with PD-L1 CPS≥1, 2 L non-operable GBM, treatment-naïve metastatic PDAC, 2 L metastatic STS, and treatment-naïve metastatic non-squamous NSCLC. Additional rationale for the chemotherapy combinations in the 3 treatment-naïve populations (Cohorts A, C, and F) is provided below.

Cohort A will evaluate the safety and preliminary efficacy of anti-ILT3 antibody in combination with pembrolizumab plus paclitaxel in treatment-naïve participants with PD-L1 CPS≥1 metastatic TNBC. Pembrolizumab in combination with standard single agent chemotherapy (paclitaxel, nab-paclitaxel, or gemcitabine/carboplatin) compared to chemotherapy alone has been evaluated as a 1 L treatment for metastatic TNBC in the randomized Phase 3 KN355 study. Pembrolizumab plus chemotherapy showed a significant improvement in PFS (9.7 months vs 5.6 months; HR: 0.65, 95% CI: 0.49-0.86) compared with chemotherapy alone in participants with PD-L1 CPS≥10 in this study (Cortes J. et al. KEYNOTE-355: randomized, double-blind, phase III study of pembrolizumab+chemotherapy versus placebo+chemotherapy for previously untreated locally recurrent inoperable or metastatic triple-negative breast cancer [abstract]. Presented at: 2020 American Society of Clinical Oncology (ASCO) Virtual Scientific Program; 2020 May 29-31; [online meeting]. J Clin Oncol. 2020; 38(15 suppl). Abstract no. 1000). Internal data from this study also show a promising ORR with the combination of pembrolizumab and paclitaxel. As paclitaxel is a widely available standard therapy for 1 L metastatic TNBC, the standard dose and schedule will be used in Cohort A, which is the same starting dose and schedule used in KN355.

Cohort C will evaluate the safety and preliminary efficacy of anti-ILT3 antibody in combination with pembrolizumab, nab-paclitaxel, and gemcitabine in treatment-naïve participants with PDAC. The combination of nab-paclitaxel and gemcitabine is a standard of care regimen for first-line treatment of patients with PDAC and is generally better tolerated than FOLFIRINOX (Von Hoff D D et al. Increased survival in pancreatic cancer with nab-paclitaxel plus gemcitabine. N Engl J Med. 2013 Oct. 31; 369(18): 1691-703; Conroy T et al. FOLFIRINOX versus gemcitabine for metastatic pancreatic cancer. N Engl J Med. 2011 May 12; 364(19):1817-25). The triplet combination of a PD-1 inhibitor with nab-paclitaxel and gemcitabine also has a tolerable safety profile in PDAC, as seen in studies with pembrolizumab and nivolumab (Weiss G J et al. A phase Ib study of pembrolizumab plus chemotherapy in patients with advanced cancer (PembroPlus). Br J Cancer. 2017 Jun. 27; 117(1):33-40; Wainberg Z A et al. Open-label, phase I study of nivolumab combined with nab-paclitaxel plus gemcitabine in advanced pancreatic cancer. Clin Cancer Res. 2020 Sep. 15; 26(18):4814-22).

Cohort E will evaluate the safety of adding anti-ILT3 antibody to the combination of pembrolizumab, carboplatin, and pemetrexed in treatment-naïve participants with metastatic non-squamous NSCLC. A platinum doublet with pemetrexed is the most commonly used 1L chemotherapy for chemotherapy-naïve metastatic non-squamous NSCLC patients. Based on results of KN021G, pembrolizumab in combination with carboplatin and pemetrexed is approved by FDA as 1 L, treatment of patients with metastatic non-squamous NSCLC, regardless of PD-L1 status. These results have been confirmed in the randomized Phase 3 KN189 study (Gandhi L et al. Pembrolizumab plus chemotherapy in metastatic non-small-cell lung cancer. N Engl J Med. May 31; 378(22):2078-2092). The doublet chemotherapy for participants in Cohort E is the standard of care regimen for non-squamous NSCLC. If safety and tolerability is demonstrated in Cohort E, the combination may be further evaluated.

### Rationale for Starting and Maximum Dose of Anti-ILT3 Antibody

The human starting dose and dosing interval of anti-ILT3 antibody are based on an integration of nonclinical toxicological, pharmacological, and pre-clinical efficacy data.

Due to the potentially immune-activating mechanism of action of anti-ILT3 antibody, the FIH starting dose of anti-ILT3 antibody of 0.2 mg (equivalent to 0.003 mg/kg for a 70 kg patient) is determined factoring in an integration of the comprehensive nonclinical pharmacology, toxicology data, and quantitative modeling.

Rhesus monkey was selected as a pharmacologically relevant species for preclinical studies.

Successful target engagement in rhesus monkeys was indicated by an observed dose-dependent increase in total soluble ILT3 levels across the dose range of 0.3 mg/kg to 30 mg/kg (Studies 17-M100-8994 and 18-M100-9870). The observed NOAEL in a multiple dose study in rhesus monkeys (dosed once every week for a total of 4 doses) was 100 mg/kg/week. In a SK-MEL-5 tumor-bearing humanized mouse model, increases in the immune activation marker HLA-DR were observed systemically in blood at a dose of 0.1 mg/kg and at the site of action (tumor) at a dose of 1.0 mg/kg. Based on the results from the SK-MEL-5 tumor bearing humanized mouse model and comparison of PK between mouse and human, a PAD approach based on blood HLA-DR activation, the inventors determined that a starting dose of 0.03 mg/kg would be appropriate. However, due to the lack of a fully human immune repertoire in the humanized mouse model, a more conservative dose may be warranted, as the mouse tumor model could be less sensitive than the patient setting. Therefore, a 0.003 mg/kg, 10-fold lower dose than PAD, is proposed as the FIH starting dose.

Allometric scaling was used to predict human PK parameters from those determined in rhesus monkey. Based on analyzing the predicted Cmax of 0.081 μg/mL at 0.003 mg/kg in humans the inventors expect to provide approximately 70% target occupancy in peripheral blood based on a mechanistic PK modeling approach considering the levels of membrane ILT3 and soluble ILT3, and binding potency of anti-ILT3 antibody to primary peripheral blood CD14* monocytes and plasma soluble ILT3. A review of the immune-activating oncology products published by the researchers at the FDA has reported acceptable toxicities at FIH doses associated with up to 80% target engagement (Saber H, et al. An FDA oncology analysis of immune activating products and first-in human dose selection. Regul Toxicol Pharmacol. 2016 November; 81:448-456).

For anti-ILT3 antibody, the predicted Cmax of 0.081 μg/ml at 0.003 mg/kg also provides an 83,580-fold safety margin relative to the Cmax of 6770 μg/ml at steady state observed in rhesus monkeys at the NOAEL of 100 mg/kg. In addition, in the in vitro cytokine release assay, no overall induction of cytokine release was observed at concentrations up to 1000 μg/ml MK-0482 alone and in combination with pembrolizumab, which is approximately 12,346-fold higher than the predicted human Cmax of 0.081 μg/ml at the starting dose of 0.003 mg/kg.

Thus, the induction of cytokine release is not expected at serum concentrations achieved at a dose of 0.003 mg/kg.

For a 70 kg patient, the body weight-based dose of 0.003 mg/kg is equivalent to a fixed dose of 0.2 mg.

### Rationale for Dose Interval and Escalation Increments

Before starting the mTPI approach, initial dose escalation will proceed following an ATD to minimize the number of participants treated at potentially subtherapeutic doses of anti-ILT3 antibody. The accelerated titration part of dose escalation will treat with up to a one-half log unit dose increment from the prior dose of anti-ILT3 antibody. Based on preclinical safety data of anti-ILT3 antibody and the desire to minimize treatment of advanced cancer participants with doses that may be ineffective, in the beginning of the study, one-half log unit increments are viewed as acceptable. The accelerated titration part of dose escalation will end with the occurrence of a Grade 2 or higher non-disease-related toxicity assessed by the investigator to be possibly, probably, or definitely related to anti-ILT3 antibody administration. After the accelerated titration part of dose escalation ends, dose finding will proceed with a model-based dose mTPI approach with 3 to 14 participants treated per DL using dose increment increases of one-half log unit of the prior dose.

In cohorts of participants treated with a combination of anti-ILT3 antibody and pembrolizumab, doses of anti-ILT3 antibody used in combination with pembrolizumab will be at least 2 DL behind the monotherapy dose until the MTD/MAD for anti-ILT3 antibody monotherapy is established and will not exceed the MTD/MAD for monotherapy. Once an MTD/MAD for the monotherapy arm is established, the dose of anti-ILT3 antibody in combination with pembrolizumab may continue escalation up to that dose.

### Accelerated Titration Design

The initial dose escalation will follow an ATD. Single participants will be enrolled into sequentially escalating dose levels with up to one-half log unit increments between dose levels (e.g., 0.2 mg, 0.7 mg, and 2 mg). A range of doses is outlined in Table 11A and 11B below. The predicted target engagement at the planned ATD doses is about 70% at 0.2 mg, about 95% at 0.7 mg, and about 99% at 2 mg. As the predicted target engagement approaches full saturation at 2 mg, the transition from ATD to mTPI is planned at the next dose level of 7.5 mg.

Intermediate dose levels may be evaluated, if warranted. The dose to be tested in each cohort of participants will be communicated to the investigators or designees following the dose escalation decision for the previous dose. Enrollment of up to 3 participants per cohort is permitted upon approval by the Sponsor Medical Monitor or designee provided the interval between each of these participants is at least 24 hours. The 24-hour interval was determined based on the results from pre-clinical studies showing that there was no significant cytokine release using anti-ILT3 antibody with or without pembrolizumab. All participants enrolled at each dose level must complete the DLT period before the next dose level is initiated.

The ATD will end when at least 1 of the following occurs:
The highest dose level cohort has completed the DLT evaluation period and anti-ILT3 antibody has been determined to be safe and well tolerated in this cohort.
Occurrence of a Grade 2 or higher non-disease-related toxicity according to NCI CTCAE, version 4), if assessed by the investigator to be possibly, probably, or definitely related to anti-ILT3 antibody administration, at any dose level during the DLT period (Cycle 1).

Anytime a DLT occurs in the ATD phase, the cohort in which the DLT occurred will be expanded at this dose, per mTPI guidelines below. If no DLT occurs in the ATD phase, then the ATD phase will proceed to the mTPI phase once 1 of the above triggers is met. In such a case, the starting dose for the mTPI phase will increase by one-half log unit increment from the last ATD dose.

TABLE 11A
Doses Selected for Dose-Escalation Part 1: ATD and mTPI Design

| Dose Escalation | | | | Pembrolizumab | |
|---|---|---|---|---|---|
| Part 1 | Dose Levels | MK-0482 Dose | | Dose | Notes |
| ATD | DL1a | 0.2 | mg Q3W | NA | Due to the potentially immune- |
| (Arm 1) | DL2a | 0.7 | mg Q3W | NA | activating mechanism of action of anti- |
| | DL3a | 2 | mg Q3W | NA | ILT3 antibody, the FIH starting dose of |
| | | | | | anti-ILT3 antibody of 0.2 mg |
| | | | | | (equivalent to 0.003 mg/kg for a 70 kg |
| | | | | | patient) is justified based on an |
| | | | | | integrationof the comprehensive |
| | | | | | nonclinical pharmacology, toxicology |
| | | | | | data and quantitative modeling. The |
| | | | | | initial dose escalation will follow an |
| | | | | | ATD. Single participants will be |
| | | | | | enrolled into sequentially escalating |
| | | | | | dose levels with up to one-half log unit |
| | | | | | increments between dose levels (e.g., |
| | | | | | 0.2 mg, 0.7 mg, and 2 mg). |

mTPI Design

| | | | | | |
|---|---|---|---|---|---|
| Arm 1 | DL1 | 7.5 | mg Q3W | NA | The predicted target engagement at the |
| | DL2 | 25 | mg Q3W | NA | planned ATD doses is about 70% at 0.2 |
| | DL3 | 75 | mg Q3W | NA | mg, about 95% at 0.7 mg, and |
| | DL4 | 225 | mg Q3W | NA | about 99% at 2 mg. As the predicted |
| | DL5 | 750 | mg Q3W | NA | target engagement approaches full |
| | DL6^{a} | 2250 | mg Q3W | NA | saturation at 2 mg, the transitionfrom |
| | | | | | ATD to mTPI is planned at the next |
| | | | | | dose level of 7.5 mg. |

TABLE 11B
Doses Selected for Dose-Escalation Part 1

Escalation

| Part 1 | Dose Levels | MK-0482 Dose | | Pembrolizumab Dose | | Notes |
|---|---|---|---|---|---|---|
| Arm 2 | DL1 | 7.5 | mg Q3W | 200 | mg Q3W | Pembrolizumab 200 mg |
| | DL2 | 25 | mg Q3W | 200 | mg Q3W | Q3W is the standard |
| | DL3 | 75 | mg Q3W | 200 | mg Q3W | dosing regimen. See |
| | DL4 | 225 | mg Q3W | 200 | mg Q3W | above for rationale |
| | DL5 | 750 | mg Q3W | 200 | mg Q3W | for anti-ILT3 |
| | DL6^{a} | 2250 | mg Q3W | 200 | mg Q3W | antibody dosing |
| | | | | | | regimen. |

Arm 1: anti-ILT3 antibody as Monotherapy Dose Escalation (ATD followed by mTPI Design)
Arm 2: anti-ILT3 antibody in Combination with Pembrolizumab 200 mg Q3W Dose Escalation (mTPI Design)
^{a}Based on review of preliminary PK, receptor occupancy, and sILT3 levels up to the 750 mg dose (DL5), an additional 3-fold higher dose (DL6) was exploredto better characterize the PK profile and pharmacodynamics of anti-ILT3 antibody and facilitate selection of the preliminary RP2D.
Abbreviations:
a = ATD dose level;
ATD = accelerated titration design;
DL = dose level;
mTPI = modified toxicity probability interval;
Q3W = every 3 weeks

### Rationale for Fixed Dose of Pembrolizumab

The planned dose of pembrolizumab for this study is 200 mg Q3W. Based on the totality of data generated in the KEYTRUDA development program, 200 mg Q3W is the appropriate dose of pembrolizumab for adults across all indications and regardless of tumor type. As outlined below, this dose is justified by:
Clinical data from 8 randomized studies demonstrating flat dose- and exposure-efficacy relationships from 2 mg/kg Q3W to 10 mg/kg Q2W,
Clinical data showing meaningful improvement in benefit-risk including overall survival at 200 mg Q3W across multiple indications, and
Pharmacology data showing full target saturation in both systemic circulation (inferred from PK data) and tumor (inferred from PBPK analysis) at 200 mg Q3W.

Among the 8 randomized dose-comparison studies, a total of 2262 participants were enrolled with melanoma and NSCLC, covering different disease settings (treatment-naïve, previously treated, PD-L1 enriched, and all-comers) and different treatment settings (monotherapy and in combination with chemotherapy). Five studies compared 2 mg/kg Q3W versus 10 mg/kg Q3W (KN001 Cohort B2, KN001 Cohort D, KN002, KN010, and KN021), and 3 studies compared 10 mg/kg Q3W versus 10 mg/kg Q2W (KN001 Cohort B3, KN001 Cohort F2 and KN006). All studies demonstrated flat dose- and exposure-response relationships across the doses studied representing an approximate 5- to 7.5 fold difference in exposure.

The 2 mg/kg (or 200 mg fixed-dose) Q3W provided similar responses to the highest doses studied. Subsequently, flat dose-exposure-response relationships were also observed in other tumor types including head and neck cancer, bladder cancer, gastric cancer and classical Hodgkin Lymphoma, confirming 200 mg Q3W as the appropriate dose independent of the tumor type. These findings are consistent with the mechanism of action of pembrolizumab, which acts by interaction with immune cells, and not via direct binding to cancer cells.

Additionally, pharmacology data clearly show target saturation at 200 mg Q3W. First, PK data in KN001 evaluating target-mediated drug disposition conclusively demonstrated saturation of PD-1 in systemic circulation at doses much lower than 200 mg Q3W. Second, a PBPK analysis was conducted to predict tumor PD-1 saturation over a wide range of tumor penetration and PD-1 expression. This evaluation concluded that pembrolizumab at 200 mg Q3W achieves full PD-1 saturation in both blood and tumor.

Finally, population PK analysis of pembrolizumab, which characterized the influence of body weight and other participant covariates on exposure, has shown that the fixed-dosing provides similar control of PK variability as weight based dosing, with considerable overlap in the distribution of exposures from the 200 mg Q3W fixed dose and 2 mg/kg Q3W dose. Supported by these PK characteristics and given that fixed-dose has advantages of reduced dosing complexity and reduced potential of dosing errors, the 200 mg Q3W fixed-dose was selected for evaluation across all pembrolizumab protocols.

### Rationale for Anti-ILT3 Antibody Preliminary RP2D

Anti-ILT3 antibody is well tolerated as a monotherapy and in combination with pembrolizumab up to a dose of anti-ILT3 antibody 2250 mg Q3W. As of 9 Nov. 2020, there were 29 participants in Arm 1 (anti-ILT3 antibody monotherapy) and 40 participants in Arm 2 (anti-ILT3 antibody in combination with pembrolizumab) who have received at least 1 dose of study intervention. Six participants from Arm 1 crossed over to Arm 2. There were no Grade 4 or Grade 5 treatment-related AEs in either arms. There was one Grade 3 treatment-related AE (pyrexia) in Arm 1 and two treatment-related Grade 3 AEs (AST elevation and adrenal insufficiency) in Arm 2. No DLTs were observed in Arm 1. One DLT was observed in Arm 2, which was a Grade 2 treatment-related myositis experienced by a participant in the anti-ILT3 antibody 2250 mg DL during Cycle 1.

This treatment-related AE led to treatment discontinuation. Most treatment-related AEs were Grade 1 or Grade 2 and the overall incidence of treatment-related AEs in the Arm 2 was nearly twice that in Arm 1 (60.9% vs. 34.5%). The most common (>5%) treatment-related AEs in Arm 2 included fatigue (17.4%), hyperthyroidism (10.9%), hypothyroidism (10.9%), arthralgia (10.9%), diarrhea (8.7%), influenza-like illness (6.5%), and pruritus (6.5%), which is consistent with what has been observed for pembrolizumab. Two participants in Arm 2 (one at the 7.5 mg DL and another at the 2250 mg DL) discontinued study treatment due to treatment-related AEs of Grade 3 AST elevation and Grade 2 myositis, respectively. An MTD was not achieved in either Arm 1 or Arm 2.

Preliminary Part 1 PK data show target-mediated drug disposition at lower anti-ILT3 antibody doses while linear PK was observed above the 75 mg dose level. Near complete receptor occupancy was also observed in blood samples from participants treated with anti-ILT3 antibody 75 mg and above. Even with stringent assumptions, anti-ILT3 antibody 750 mg is likely to maintain complete receptor occupancy in the tumor. While ADA was observed in 13 out of 58 participants, there was no clear impact of ADA on PK or receptor occupancy. No ADA was observed at the anti-ILT3 antibody 750 mg dose. A dose dependent increase in total soluble ILT3 concentration was seen in blood samples; however, based on Sponsor investigation, there was no confirmed immunosuppressive activity for soluble ILT3.

Based on a comprehensive evaluation of data, anti-ILT3 antibody 750 mg Q3W in combination with pembrolizumab 200 mg Q3W is the preliminary RP2D for further evaluation in the expansion cohorts.

### Rationale for Paclitaxel Dose for Cohort A

Paclitaxel is a widely available standard therapy for 1L metastatic TNBC. The standard dose and schedule, which is the same dose and schedule used in Study KN355, will be used in Cohort A. Participants will receive paclitaxel 90 mg/m² by IV infusion Days 1, 8, and 15 every 28 days until PD or unacceptable toxicity that requires discontinuation.

### Rationale for Nab-Paclitaxel and Gemcitabine Doses for Cohort C

The doublet chemotherapy for participants in Cohort C is a standard of care regimen for 1L metastatic PDAC. Participants will receive nab-paclitaxel 125 mg/m² by IV infusion followed by gemcitabine 1000 mg/m² by IV infusion on Days 1, 8, and 15 every 28 days until PD or unacceptable toxicity that requires discontinuation.

### Rationale for Carboplatin and Pemetrexed Doses for Cohort E

The doublet chemotherapy for participants in Cohort E is a standard of care regimen for 1L non-squamous NSCLC. Participants will receive carboplatin AUC 5 and pemetrexed 500 mg/m², both administered by IV infusion Q3W for 4 cycles, followed by maintenance therapy with pemetrexed for up to a total of 35 cycles.

### Study Population

Male/female participants at least 18 years of age with advanced/metastatic solid tumors will be enrolled in this study.

Prospective approval of protocol deviations to recruitment and enrollment criteria, also known as protocol waivers or exemptions, is not permitted.

### Inclusion Criteria—Cohort A: TNBC

A participant will be eligible for inclusion in the study if the participant:
Has histologically confirmed locally recurrent inoperable or metastatic TNBC, as defined by the most recent ASCO/CAP guidelines.
Has received no prior systemic therapy for metastatic TNBC.
   Note: If with recurrent disease from prior Stage I-III TNBC, ≥6 months must have elapsed between completion of treatment with curative intent (e.g., date of primary breast tumor surgery or date of last neoadjuvant or adjuvant systemic treatment, whichever occurred last) and the first documented local or distant disease recurrence (via biopsy, pathology report or imaging report).
   Note: Adjuvant radiation therapy is not considered treatment with curative intent for the purpose of calculating the ≥6 month interval requirement described above.
Has tumor PD-L1 CPS≥1 as assessed by the designated central laboratory.

### Inclusion Criteria—Cohort B: GBM

A participant will be eligible for inclusion in the study if the participant:
Has histologically confirmed diagnosis of GBM (WHO Grade IV malignant glioblastoma).
Has received a standard first-line treatment for GBM including surgery (resection) and radiation therapy with or without chemotherapy and shown unequivocal evidence of disease recurrence or tumor progression by MRI. The disease should be considered non-operable. Only participants with 2 L GBM are eligible.
Has time intervals elapsed for the following activities before the start of study treatment:
   at least 3 weeks from prior surgical resection,
   at least 1 week from prior stereotactic biopsy, and
   at least 12 weeks from completion of radiation therapy unless there is unequivocal histologic confirmation of tumor progression or radiographic progression outside of the prior radiation field.
Has a KPS≥80 within 7 days before the start of study treatment.
Is neurologically stable (e.g., without a progression of neurological symptoms or requiring escalating doses of systemic steroid therapy within the last 2 weeks) and clinically stable.
Has known status of MGMT methylation (methylated or unmethylated) and IDH (wild type or with mutation).

### Inclusion Criteria—Cohort C: PDAC

A participant will be eligible for inclusion in the study if the participant:
Has histologically confirmed diagnosis of metastatic PDAC.
Has received no prior systemic therapy for metastatic PDAC including chemotherapy, biological therapy, or targeted therapy.
   Note: Participants who have received prior adjuvant or neoadjuvant systemic therapy (including gemcitabine, nab-paclitaxel, other chemotherapy) for non-metastatic PDAC are eligible if treatment was completed for more than 4 months before the start of study treatment.
   Note: Palliative radiotherapy is allowed if it was completed at least 2 weeks before the start of study treatment.
Has albumin ≥3.0 g/dL.
   Inclusion Criteria—Cohort D: mSTS

A participant will be eligible for inclusion in the study if the participant:
Has a histologically confirmed diagnosis of locally advanced or metastatic STS.
   Note: Patients with diagnosis of GIST are excluded.
Has received and progressed after one prior line of systemic treatment for advanced STS. Only participants with 2 L STS are eligible.

### Inclusion Criteria—Cohort D: NSCLC

A participant will be eligible for inclusion in the study if the participant:
Has histologically confirmed diagnosis of Stage IV or recurrent non-operable non-squamous NSCLC.
Has confirmation that EGFR-, ALK-, or ROS1-directed therapy is not indicated as primary therapy (documentation of absence of tumor activating EGFR and absence of ALK or ROS1 gene rearrangements is required).
Has not received prior systemic treatment for metastatic NSCLC.
   Note: Participants who received adjuvant or neoadjuvant therapy are eligible if the adjuvant/neoadjuvant therapy was completed at least 6 months prior to the documented local or distant disease recurrence.

Additional umbrella studies co-administering anti-ILT3 antibody with pembrolizumab in other NSCLC patient sub-populations will have additional inclusion criteria. In such umbrella studies, pembrolizumab will be administered prior to anti-ILT3 antibody at a dose of 200 mg using a 30-minute IV infusion Q3W, and anti-ILT3 antibody will be administered at a dose of 750 mg using a 30-minute IV infusion Q3W, for a maximum of 35 cycles (approximately 2 years).

In a first umbrella study, subjects have refractory non-squamous NSCLC after chemotherapy. In the study, a subject will be eligible for inclusion if the subject:
Has a histologically- or cytologically-confirmed diagnosis of Stage IV squamous or non-squamous NSCLC
Has non-squamous NSCLC and is not eligible for an approved targeted therapy
Is able to provide archival tumor tissue sample collected either within 5 years or within the interval from completion of last treatment but before entering the screening period or newly obtained core or excisional biopsy of a tumor lesion not previously irradiated obtained within 90 days of treatment initiation
Has progressed on treatment with an anti-PD-(L)1 monoclonal antibody (mAb) administered either as monotherapy, or in combination with other checkpoint inhibitors or other therapies
Has progressive disease (PD) during/after platinum doublet chemotherapy
Is able to complete all screening procedures within the 35-day screening window
Male participants must agree to use contraception and refrain from donating sperm during the treatment period and for at least 120 days after the last dose of study treatment
Female participants must not be pregnant or breastfeeding, and at least one of the following conditions apply:
   Not a woman of childbearing potential (WOCBP) OR
   A WOCBP who agrees to use contraception during the treatment period and for at least 120 days after the last dose of study treatment
Has adequate organ function within 10 days of initiation of study treatment

In a second umbrella study, subjects have non-squamous NSCLC and have not received prior systemic treatment for mNSCLC. In the study, a subject will be eligible for inclusion if the subject:
Has histologically confirmed diagnosis of Stage IV or recurrent non-operable non-squamous NSCLC.
Has confirmation that EGFR-, ALK-, or ROS1-directed therapy is not indicated as primary therapy (documentation of absence of tumor activating EGFR and absence of ALK or ROS1 gene rearrangements is required).
Has not received prior systemic treatment for metastatic NSCLC.
Has tumor PD-L1 CPS≥1 as assessed by the designated central laboratory.

### Exclusion Criteria—All Participants

The participant must be excluded from the study if the participant:

### Medical Conditions

Has a history of a second malignancy, unless potentially curative treatment has been completed with no evidence of recurrence for at least 2 years.
   Note: The time requirement does not apply to participants who underwent successful definitive resection of basal cell carcinoma of the skin, squamous cell carcinoma of the skin, superficial bladder cancer, or carcinoma in situ (e.g., breast carcinoma in situ, cervical cancer in situ).
Has known active central nervous system metastases (i.e., brain and/or spinal cord) and/or carcinomatous meningitis. Participants with previously treated brain metastases may participate provided they are radiologically stable and asymptomatic (i.e., without evidence of progression by MRI for at least 4 weeks by repeat imaging (note that the repeat imaging should be performed during study screening), have no evidence of new or enlarging brain metastases, are evaluated within 4 weeks before the start of study treatment, and are off immunosuppressive doses of systemic steroid treatment for at least 14 days before the start of study treatment. This eligibility criterion does not apply to Cohort B.
Has had a severe hypersensitivity reaction to treatment with a mAb and/or any component of pembrolizumab or anti-ILT3 antibody.
Has received any prior immunotherapy and was discontinued from that treatment due to a Grade 3 or higher irAE.
Has an active infection requiring systemic therapy.
Has a history of interstitial lung disease.
Has a history of (non-infectious) pneumonitis that required steroids or has current pneumonitis.
Has an active autoimmune disease that has required systemic treatment in the past 2 years (i.e., with use of disease modifying agents, corticosteroids or immunosuppressive drugs), except vitiligo or resolved childhood asthma/atopy. Replacement therapy (e.g., thyroxine, insulin, or physiologic corticosteroid replacement therapy for adrenal or pituitary insufficiency) is not considered a form of systemic treatment and is allowed.
   Note: Participants receiving ongoing replacement hormone therapy for endocrine insufficiency will not be excluded from participation in the study, if the associated deficiency has recovered to NCI CTCAE, version 4 Grade 2 with replacement hormone therapy prior to the first dose of study treatment. Use of non-systemic steroids is also permitted.
HIV-infected participants with a history of Kaposi's sarcoma and/or Multicentric Castleman's Disease.
Has known Hepatitis B or C infections or known to be positive for hepatitis B antigen/hepatitis B virus DNA or hepatitis C antibody and RNA. Active hepatitis C is defined by a known positive hepatitis C antibody result and known quantitative hepatitis C virus RNA results greater than the lower limits of detection of the assay.
   Note: No testing for hepatitis B or hepatitis C is required unless mandated by local health authority.
Has a history or current evidence of any condition, therapy, or laboratory abnormality that might confound the results of the study, interfere with the participant's participation for the full duration of the study, make administration of the study drugs hazardous, or make it difficult to monitor adverse effects such that it is not in the best interest of the participant to participate, in the opinion of the treating investigator.
Has a known psychiatric or substance abuse disorder that would interfere with the participant's ability to cooperate with the requirements of the study.

### Prior/Concomitant Therapy

Has received prior systemic anticancer therapy, definitive radiotherapy, including investigational agents within 4 weeks (2 weeks for palliative radiation) before the start of study treatment.
   Note: Participants must have recovered from all AEs due to previous therapies to ≤Grade 1 or baseline.
   Note: STS candidates for Cohort D can be eligible if 1 L standard chemotherapy was discontinued >3 weeks before the start of study treatment.
Has had major surgery (<3 weeks before the start of study treatment)
   Note: If participant received major surgery, they must have recovered adequately from the toxicity and/or complications from the intervention prior to starting study treatment.
Has received a live vaccine within 30 days prior to the start of study treatment. Examples of live vaccines include, but are not limited to, the following: measles, mumps, rubella, varicella/zoster (chicken pox), yellow fever, rabies, Bacillus Calmette-Guérin (BCG), and typhoid vaccine. Seasonal influenza vaccines for injection are generally killed virus vaccines and are allowed; however, intranasal influenza vaccines (e.g., FLUMIST) are live attenuated vaccines and are not allowed.
Has received prior treatment(s) with another agent targeting ILT3.

### Prior/Concurrent Clinical Study Experience

Is currently participating in or has participated in a study of an investigational agent or has used an investigational device within 28 days before the start of study treatment.
   Note: Participants who have entered the follow-up phase of an investigational study may participate as long as it has been 4 weeks after the last dose of the previous investigational agent.

### Diagnostic Assessments

Has a diagnosis of immunodeficiency or is receiving chronic systemic steroid therapy (in dosing exceeding 10 mg daily of prednisone equivalent) or any other form of immunosuppressive therapy within 7 days before the start of study treatment.
   Note: Participants that require intermittent use of non-systemic steroids such as ocular, inhaled, intranasal, topical steroids, or local steroid injections are not excluded from the study.

### Other Exclusions

Has had an allogeneic tissue/solid organ transplant in the last 5 years or has evidence of graft-versus-host disease.

### Exclusion Criteria-Cohort Specific

Has received prior therapy with an anti-PD-1, anti-PD-L1, or anti-PD-L2 agent or prior therapy targeting other immune-regulatory receptors or mechanisms.
   Note, if any of the above immunotherapy was received as neoadjuvant or adjuvant treatment with curative intent for localized disease (Stage I to III), a candidate participant can be eligible if >12 months have elapsed.

### Cohort A: TNBC

Has a history of class II-IV congestive heart failure or myocardial infarction within 6 months of the start of study treatment.
Has a known sensitivity to any component of paclitaxel or any of its excipients.
Is receiving any medication prohibited in combination with paclitaxel as described in the product label unless medication was stopped within 7 days before the start of study treatment.

### Cohort B: GBM

Has carcinomatous meningitis.
Has recurrent tumor greater than 6 cm in maximum diameter.
Has tumor primarily localized to the brainstem or spinal cord.
Has presence of multifocal tumor, diffuse leptomeningeal or extracranial disease.
Has evidence of intratumoral or peritumoral hemorrhage on baseline MRI scan other than those that are ≤Grade 1 and either post-operative OR stable on at least 2 consecutive MRI scans.
Requires treatment with moderate or high dose systemic corticosteroids defined as dexamethasone >2 mg/day or bioequivalent for at least 3 consecutive days within 2 weeks of start of study treatment.
Has received OPTUNE TTFields within 2 weeks of start of study treatment.

### Cohort C: PDAC

Has a history of class II-IV congestive heart failure, cerebral vascular event (stroke or transient ischemic attack), unstable angina, or myocardial infarction within 6 months of the start of study treatment.
Has symptomatic ascites.
Has a known hypersensitivity to nab-paclitaxel or gemcitabine, or any of their excipients.

### Cohort E: NSCLC

Has a diagnosis of small cell lung cancer. For mixed tumors, if small cell elements are present, the participant is ineligible.
Has symptomatic ascites or pleural effusion.
   Note: A participant who is clinically stable following treatment for these conditions (including therapeutic thoracentesis or paracentesis) is eligible.
Is currently receiving either strong or moderate inhibitors and/or inducers of CYP3A4 or CYP2C8 that cannot be discontinued for the duration of the study. The required washout period before the start of study treatment for CYP3A4 or CYP2C8 inhibitors is 2 weeks; the required washout period for CYP3A4 or CYP2C8 inducers is 3 weeks.
   Note: A current list of strong/moderate inducers and inhibitors of CYP3A4 can be found at the following website: www.fda.gov/drugs/drug-interactions-labeling/drug-development-and-drug-interactions-table-substrates-inhibitors-and-inducers
Is unable to interrupt aspirin or other NSAIDs, other than aspirin dose≤1.3 g/day for a 5-day period (8-day period for long acting agents such as peroxicam).
Is unable or unwilling to take folic acid or vitamin B12 supplementation.
Has a known hypersensitivity to carboplatin or pemetrexed, or any of their excipients.

### Anti-ILT3 Antibody Administration

Anti-ILT3 antibody will be administered at the dose level assigned in the specific arm or cohort as IV infusion or bolus administration Q3W according to the pharmacy manual. In Part 1 Arm 2 and Part 2 cohorts, anti-ILT3 antibody will be administered after completion of the pembrolizumab infusion on the days when pembrolizumab is administered, if applicable. The reason for any variability in the administration of anti-ILT3 antibody outside of the protocol-specified window should be documented in the participant's chart and recorded on the appropriate CRF. Study treatment should begin within 3 days of treatment allocation. All study treatments will begin on Day 1 of each cycle after all pre-dose study procedures and assessments have been completed and results reviewed by the investigator or designee.

### Pembrolizumab Administration

Pembrolizumab will be administered prior to anti-ILT3 antibody at a dose of 200 mg using a 30-minute IV infusion Q3W. For both pembrolizumab and anti-ILT3 antibody, sites should make every effort to target administration timing to be as close as possible to the duration(s) outlined in the pharmacy manual.

### Paclitaxel Administration

Paclitaxel 90 mg/m² will be administered as an IV infusion Days 1, 8, and 15 every 28 days. All participants should be premedicated with oral or IV corticosteroid and anti-histamines according to the approved product label and/or standard practice. Additional premedications should be administered as per standard practice.

On Day 1 of each 21-day cycle, paclitaxel will be administered after completion of the pembrolizumab and anti-ILT3 antibody infusions.

### Nab-Paclitaxel Administration

Nab-paclitaxel 125 mg/m² will be administered as an IV infusion Days 1, 8, and 15 every 28 days. Nab-paclitaxel should be administered according to the approved product label and/or standard practice.

On Day 1 of each 21-day cycle, nab-paclitaxel will be administered after completion of the pembrolizumab and anti-ILT3 antibody infusions.

### Gemcitabine Administration

Gemcitabine 1000 mg/m² will be administered as an IV infusion Days 1, 8, and 15 every 28 days. Gemcitabine should be administered according to the approved product label and/or standard practice.

On Day 1 of each 21-day cycle, gemcitabine will be administered after completion of the pembrolizumab and anti-ILT3 antibody infusions.

### Pemetrexed Administration

Pemetrexed 500 mg/m² will be administered as an IV infusion Q3W for 35 cycles.

Pemetrexed should be administered after completion of the pembrolizumab and anti-ILT3 antibody infusions and before carboplatin.

Participants will receive the appropriate premedications (folic acid supplementation, vitamin B12 supplementation, and dexamethasone prophylaxis) in accordance with local regulations.

### Carboplatin Administration

Carboplatin AUC 5 mg/mL·min will be administered as an IV infusion Q3W for 4 cycles. Carboplatin should be administered immediately after pemetrexed administration as per local practice and labels.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references cited herein are incorporated by reference to the same extent as if each individual publication, database entry (e.g., Genbank or GeneID entries), patent application, or patent, was specifically indicated to be incorporated by reference. This statement is intended by Applicants, pursuant to 37 C.F.R. § 1.57(b)(1), to relate to each and every individual publication, database entry (e.g., Genbank or GeneID entries), patent application, or patent, each of which is clearly identified in compliance with 37 C.F.R. § 1.57(b)(2), even if such citation is not immediately adjacent to a dedicated statement of incorporation by reference. Citation of the references herein is not intended as an admission that the reference is pertinent prior art, nor does it constitute any admission as to the contents or date of these publications or documents.

What is claimed is:

1. A pharmaceutical composition comprising from 0.02 mg to 2250 mg of an anti-ILT3 antigen binding protein or antigen binding fragment and a pharmaceutically acceptable excipient.

2. A method of treating cancer in a subject in need thereof comprising administering to the subject the pharmaceutical composition of claim 1.

3. The method of claim 2, comprising further administering to the subject a therapeutically effective dose of an anti-PD1 antigen binding protein, or antigen binding fragment serially or simultaneously with the pharmaceutical composition.

4. The method of any one of claims 2-3, wherein the cancer is metastatic triple negative breast cancer (mTNBC).

5. The method of claim 4, wherein before the administration step, the subject is identified as:
a) having a PD-L1 enriched tumor, wherein the PD-L1 enriched tumor is a tumor identified as having a CPS score of ≥1; and
b) having received no prior systemic therapy for mTNBC.

6. The method of any one of claims 2-3, wherein the cancer is recurrent non-operable glioblastoma multiforme (GBM).

7. The method of claim 6, wherein, before the administration step, the subject is identified as:
a) having a histologically confirmed diagnosis of GBM; and
b) having received a standard first-line treatment for GBM including surgery and radiation therapy with or without chemotherapy and evidence of disease recurrence or tumor progression by magnetic resonance imaging (MRI).

8. The method of any one of claims 2-3, wherein the cancer is metastatic pancreatic ductal adenocarcinoma (mPDAC).

9. The method of claim 8, wherein before the administration step, the subject is identified as:
a) having a histologically confirmed diagnosis of mPDAC; and
b) having received no prior systemic therapy for mPDAC.

10. The method of any one of claims 2-3, wherein the cancer is metastatic soft tissue sarcoma (mSTS).

11. The method of claim 10, wherein before the administration step, the subject is identified as:
a) having a histologically confirmed diagnosis of locally advanced or metastatic mSTS; and
b) having progressed after receiving one prior line of systemic treatment for advanced mSTS.

12. The method of any one of claims 2-3, wherein the cancer is metastatic non-squamous non-small cell lung carcinoma (mNSCLC).

13. The method of claim 12, wherein before the administration step, the subject is identified as:
a) having a histologically confirmed diagnosis of Stage IV or recurrent non-operable non-squamous non-small cell lung carcinoma (NSCLC);
b) not having epidermal growth factor receptor (EGFR), anaplastic lymphoma kinase (ALK), or c-ros oncogene 1 (ROS1) directed therapy indicated as a primary therapy; and
c) not having received prior systemic treatment for metastatic NSCLC.

14. The method of claim 12, wherein before the administration step, the subject is identified as:
a) having a histologically confirmed diagnosis of Stage IV or recurrent non-operable non-squamous non-small cell lung carcinoma (NSCLC);
b) not eligible for an approved targeted therapy;
c) having progressed on treatment with an anti-PD-(L)1 monoclonal antibody (mAb) administered either as monotherapy, or in combination with other checkpoint inhibitors or other therapies; and
d) having progressive disease (PD) during/after platinum doublet chemotherapy.

15. The method of claim 12, wherein before the administration step, the subject is identified as:
a) having a histologically confirmed diagnosis of Stage IV or recurrent non-operable non-squamous non-small cell lung carcinoma (NSCLC);
b) not having epidermal growth factor receptor (EGFR), anaplastic lymphoma kinase (ALK), or c-ros oncogene 1 (ROS1) directed therapy indicated as a primary therapy;
c) not having received prior systemic treatment for metastatic NSCLC; and
d) having a PD-L1 enriched tumor, wherein the PD-L1 enriched tumor is a tumor identified as having a CPS score of ≥1.

16. The method of any one of claims 2-15, wherein the subject is a human.

17. The method of any one of claims 2-16 or the pharmaceutical composition of claim 1, wherein the anti-ILT3 antigen-binding protein or antigen-binding fragment is an anti-ILT3 antibody or antigen-binding fragment.

18. The method or pharmaceutical composition of claim 17, wherein the antibody or antigen binding fragment that binds human immunoglobulin-like transcript 3 (ILT3) comprising:
a heavy chain (HC) having a variable heavy domain (VH) comprising a complementarity determining region (HC-CDR) 3 having an amino acid sequence selected from the group consisting of SEQ ID NO: 20, 47, 55, 63, 71, 79, 87, 95, and 103, or having an amino acid sequence that has 3, 2, or 1 differences with an amino acid sequence selected from the group consisting of SEQ ID NO: 20, 47, 55, 63, 71, 79, 87, 95, and 103.

19. The method or pharmaceutical composition of claim 17, wherein the anti-ILT3 antibody or antigen binding fragment comprises:
(a) a heavy chain (HC) having a variable heavy domain (VH) comprising a complementarity determining region (HC-CDR) 1 having the amino acid sequence set forth in SEQ ID NO: 15, 45, 53, 61, 69, 77, 85, 93, or 101; an HC-CDR2 having the amino acid sequence set forth in SEQ ID NO: 16, 46, 54, 62, 69, 78, 86, 94, or 102; and an HC-CDR3 having the amino acid sequence set forth in SEQ ID NO: 21, 47, 55, 63, 71, 79, 87, 95, or 103; and, variants thereof wherein one or more of the HC-CDRs has one, two, or three amino acid substitutions, additions, deletions, or combinations thereof; and
(b) a light chain (LC) having a variable light domain (VL) comprising a complementarity determining region (LC-CDR) 1 having the amino acid sequence set forth in SEQ ID NO: 25, 48, 56, 64, 72, 80, 88, 96, or 104; an LC-CDR2 having the amino acid sequence set forth in SEQ ID NO: 41, 49, 57, 65, 73, 81, 89, 97, or 105; and an LC-CDR3 having the amino acid sequence set forth in SEQ ID NO: 42, 50, 58, 66, 74, 82, 90, 98, or 106; and, variants thereof wherein one or more of the LC-CDRs has one, two, or three amino acid substitutions, additions, deletions, or combinations thereof.

20. The method or pharmaceutical composition of claim 19, wherein
(a) the HC-CDR1 has the amino acid sequence set forth in SEQ ID NO: 15; the HC-CDR2 has the amino acid sequence set forth in SEQ ID NO: 17, 18, or 19; the HC-CDR3 has the amino acid sequence set forth in SEQ ID NO: 21; and
(b) the LC-CDR1 has the amino acid sequence set forth in SEQ ID NO: 32, 33, 34, 35, 36, 37, 38, 39, or 40; the LC-CDR2 has the amino acid sequence set forth in SEQ ID NO: 41; and, the LC-CDR3 has the amino acid sequence set forth in SEQ ID NO: 42.

21. The method or pharmaceutical composition of claim 20, wherein
(a) the HC-CDR1 has the amino acid sequence set forth in SEQ ID NO: 15; the HC-CDR2 has the amino acid sequence set forth in SEQ ID NO: 18; and the HC-CDR3 has the amino acid sequence set forth in SEQ ID NO: 21; and
(b) the LC-CDR1 has the amino acid sequence set forth in SEQ ID NO: 39; the LC-CDR2 has the amino acid sequence set forth in SEQ ID NO: 41; and, the LC-CDR3 has the amino acid sequence set forth in SEQ ID NO: 42.

22. The method or pharmaceutical composition of any one of claims 19-21, wherein the V_{H} comprises a framework selected from the group consisting of human V_{H}1, V_{H}2, V_{H}3, V_{H}4, V_{H}5, and V_{H}6, and variants thereof having 1, 2, 3, 4, 5, 6, 7, 8, 9, or 10 amino acid substitutions, additions, deletions, or combinations thereof; and, the V_{L} comprises a framework selected from the group consisting of human V_{κ}1, V_{κ}2, V_{κ}3, V_{κ}4, V_{κ}5, V_{κ}6, V_{λ}1, V_{λ}2, V_{λ}3, V_{λ}4, V_{λ}5, V_{λ}6, V_{λ}7, V_{λ}8, V_{λ}9, and V_{λ}10, and variants thereof having 1, 2, 3, 4, 5, 6, 7, 8, 9, or 10 amino acid substitutions, additions, deletions, or combinations thereof.

23. The method or pharmaceutical composition of any one of claims 19-22, wherein the antibody comprises an HC having a human IgG1, IgG2, IgG3, or IgG4 HC constant domain or variant thereof having 1, 2, 3, 4, 5, 6, 7, 8, 9, or 10 amino acid substitutions, additions, deletions, or combinations thereof compared to the amino acid sequence of the native IgG1, IgG2, IgG3, or IgG4 isotype constant domain.

24. The method or pharmaceutical composition of claim 22 or 23, wherein the antibody comprises an LC having a human kappa or lambda LC constant domain or variant thereof comprising 1, 2, 3, 4, 5, 6, 7, 8, 9, or 10 amino acid substitutions, additions, deletions, or combinations thereof compared to the amino acid sequence of the native human kappa or lambda light chain constant domain.

25. The method or pharmaceutical composition of claim 21, wherein the antibody comprises:
(i) a V_{H} having a framework selected from human V_{H}1, V_{H}2, V_{H}3, V_{H}4, V_{H}5, and V_{H}6 and a human IgG1 or IgG4 HC constant domain or variant thereof comprising 1, 2, 3, 4, 5, 6, 7, 8, 9, or 10 amino acid substitutions, additions, deletions, or combinations thereof compared to the amino acid sequence of the native IgG1 or IgG4 isotype HC constant domain; and,
(ii) a V_{L} having a framework selected from human V_{κ}1, V_{κ}2, V_{κ}3, V_{κ}4, V_{κ}5, V_{κ}6, V_{λ}1, V_{λ}2, V_{λ}3, V_{λ}4, V_{λ}5, V_{λ}6, V_{λ}7, V_{λ}8, V_{λ}9, and V_{λ}10 and a human kappa or lambda LC constant domain or variant thereof comprising 1, 2, 3, 4, 5, 6, 7, 8, 9, or 10 amino acid substitutions, additions, deletions, or combinations thereof compared to the amino acid sequence of the native human kappa or lambda LC constant domain.

26. The method or pharmaceutical composition of claim 22, wherein the antibody or antigen binding fragment comprises a V_{H} and a V_{L} having the amino acid sequences set forth in SEQ ID NO: 13 and SEQ ID NO: 14, respectively; SEQ ID NO: 43 and SEQ ID NO: 44, respectively; SEQ ID NO: 51 and SEQ ID NO: 52, respectively; SEQ ID NO: 59 and SEQ ID NO: 60, respectively; SEQ ID NO: 67 and SEQ ID NO: 68, respectively; SEQ ID NO: 75 and SEQ ID NO: 76, respectively; SEQ ID NO: 83 and SEQ ID NO: 84, respectively; SEQ ID NO: 91 and SEQ ID NO: 92, respectively; or SEQ ID NO: 99 and SEQ ID NO: 100, respectively.

27. The method or pharmaceutical composition of claim 22, wherein the antibody or antigen binding fragment comprises a V_{H} having the amino acid sequence set forth in SEQ ID NO: 115, 116, 117, 121, 122, or 123 and a V_{L} having the amino acid sequence set forth in SEQ ID NO: 124, 125, 126, 127, 128, 129, 130, 131, 132, 133, 134, 135, 136, 137, 138, or 139.

28. The method or pharmaceutical composition of claim 27, wherein the antibody or antigen binding fragment comprises a V_{H} having the amino acid sequence set forth in SEQ ID NO: 116 and a V_{L} having the amino acid sequence set forth in SEQ ID NO: 138.

29. The method or pharmaceutical composition of any one of claims 25-28, wherein the antibody comprises a heavy chain (HC) constant domain comprising the amino acid sequence set forth in SEQ ID NO: 7, 8, 9, 10, or 11.

30. The method or pharmaceutical composition of any one of claims 23-26, wherein the antibody comprises a light chain (LC) constant domain comprising the amino acid sequence set forth in SEQ ID NO: 12.

31. The method or pharmaceutical composition of any one of claims 25-28, wherein the antibody comprises a heavy chain (HC) comprising the amino acid sequence of SEQ ID NO: 140, 141, 142, 146, 147, 148, 165, 166, 167, 168, 172, 173, 174, 175, 176, 180, 181, 182, 183, 184, 185, 189, 190, or 191.

32. The method or pharmaceutical composition of any one of claims 25-31, wherein the antibody comprises a light chain (LC) comprising the amino acid sequence set forth in SEQ ID NO: 149, 150, 151, 152, 153, 154, 155, 156, 157, 158, 159, 160, 161, 162, 163, or 164.

33. The method or pharmaceutical composition of claim 25, wherein the antibody comprises a heavy chain (HC) comprising the amino acid sequence set forth in SEQ ID NO: 141 and a light chain (LC) comprising the amino acid sequence set forth in SEQ ID NO: 163, and variants thereof wherein the HC lacks a C-terminal Lysine residue or a C-terminal glycine-lysine.

34. The method of any one of claims 2-33, wherein the anti-PD1 antigen binding protein or antigen binding fragment is an anti-PD-1 antibody or antigen binding fragment.

35. The method of claim 34, wherein the anti-PD-1 antibody or antigen-binding fragment comprises:
(a) light chain complementarity determining regions (CDRs) comprising a sequence of amino acids as set forth in SEQ ID NOs: 224, 225 and 226 and heavy chain CDRs comprising a sequence of amino acids as set forth in SEQ ID NOs: 227, 228, and 229; or
(b) light chain CDRs comprising a sequence of amino acids as set forth in SEQ ID NOs: 230, 231 and 232 and heavy chain CDRs comprising a sequence of amino acids as set forth in SEQ ID NOs: 233, 234, and 235.

36. The method of any of claim 34 or 35, wherein the anti-PD-1 antibody or antigen-binding fragment comprises:
(a) a heavy chain variable region comprising a sequence of amino acids as set forth in SEQ ID NO: 236, or a variant of SEQ ID NO: 236, and
(b) a light chain variable region comprising:
(i) a sequence of amino acids as set forth in SEQ ID NO: 237, or a variant of SEQ ID NO: 237,
(ii) a sequence of amino acids as set forth in SEQ ID NO: 238, or a variant of SEQ ID NO: 238, or
(iii) a sequence of amino acids as set forth in SEQ ID NO: 239, or a variant of SEQ ID NO: 239.

37. The method of any one of claims 34-36, wherein the anti-PD-1 antibody or antigen-binding fragment comprises a heavy chain variable region comprising a sequence of amino acids as set forth in SEQ ID NO: 236 and a light chain variable region comprising a sequence of amino acids as set forth in SEQ ID NO: 237.

38. The method of any one of claims 34-37, wherein the anti-PD-1 antibody or antigen-binding fragment is a monoclonal antibody comprising:
(a) a heavy chain comprising a sequence of amino acids as set forth in SEQ ID NO: 28, or a variant of SEQ ID NO: 240, and
(b) a light chain comprising a sequence of amino acids as set forth in SEQ ID NO: 241, a variant of SEQ ID NO: 241, SEQ ID NO: 242, a variant of SEQ ID NO: 242, SEQ ID NO: 243, or a variant of SEQ ID NO: 243.

39. The method of any one of claims 34-38, wherein the anti-PD-1 antibody or antigen-binding fragment is a monoclonal antibody comprising a heavy chain comprising a sequence of amino acids as set forth in SEQ ID NO: 240 and a light chain comprising a sequence of amino acids as set forth in SEQ ID NO: 241.

40. The method of claim 39, wherein the anti-PD1 antibody or antigen binding fragment comprises heavy chain variable domain complementarity determining regions (HC-CDR) 1, 2, and 3, and light chain variable domain complementarity determining regions (LC-CDR) 1, 2, and 3, wherein:
the HC-CDR1 comprises the amino acid sequence set forth in SEQ ID NO: 249; the HC-CDR2 comprises the amino acid sequence set forth in SEQ ID NO: 250; the HC-CDR3 comprises the amino acid sequence set forth in SEQ ID NO: 251; and
the LC-CDR1 comprises the amino acid sequence set forth in SEQ ID NO: 244; the LC-CDR2 comprises the amino acid sequence set forth in SEQ ID NO: 245; and the LC-CDR3 comprises the amino acid sequence set forth in SEQ ID NO: 246.

41. The method of claim 39, wherein the anti-PD1 antibody or antigen binding fragment has a heavy chain variable region comprising the amino acid sequence set forth in SEQ ID NO: 252 and a light chain variable region comprising the amino acid sequence set forth in SEQ ID NO: 247.

42. The method of claim 39, wherein the anti-PD-1 antibody or antigen-binding fragment is a monoclonal antibody comprising a heavy chain comprising a sequence of amino acids as set forth in SEQ ID NO: 253 and a light chain comprising a sequence of amino acids as set forth in SEQ ID NO: 248.

43. The method or pharmaceutical composition of any one of claims 2-42, wherein the therapeutically effective amount of the anti-ILT3 antigen binding protein is from about 7.5 mg to about 2250 mg and the therapeutically effective amount of the anti-PD1 antigen binding protein is about 200 mg.

44. The method or pharmaceutical composition of any one of claims 2-43, wherein the therapeutically effective amount of the anti-ILT3 antigen binding protein is about 750 mg and the therapeutically effective amount of the anti-PD1 antigen binding protein is about 200 mg.

45. The method of any one of claims 2-44, wherein the anti-PD-1 antibody or antigen binding fragment and the anti-ILT3 antibody or antigen binding fragment are administered every three weeks (Q3W) of a 21-day cycle.

46. The method of any one of claims 4-45, comprising administering a taxane.

47. The method of claim 46, wherein the taxane is paclitaxel.

48. The method of claim 47, comprising administering the paclitaxel on days 1, 8 and 15 of a 28 day cycle.

49. The method of any one of claim 47 or 48, wherein the amount of paclitaxel administered on each administration day is about 90 mg/m².

50. The method of any one of claims 6-45, comprising administering nab-paclitaxel and gemcitabine.

51. The method of claim 50, comprising administering nab-paclitaxel in an amount of about 125 mg/m² via IV infusion and gemcitabine in an amount of about 1000 mg/m² via IV infusion on Days 1, 8 and 15 of a 28 day cycle.

52. The method of any one of claims 8-45, comprising administering
a) pemetrexed in an amount of about 500 mg/m² via IV infusion every three weeks (Q3W);
b) carboplatin with desired dose of area under the cure (AUC), administered via IV infusion Q3W for 4 administrations (up to about 3 months); and
c) pemetrexed in amount of about 500 mg/m², administered via IV infusion Q3W for 4 administrations (up to about 3 months), followed by maintenance therapy with pemetrexed in an amount of about 500 mg/m² via IV infusion.

53. The method of any of claims 2-52, wherein the anti-ILT3 antibody or antigen-binding fragment is administered to the patient by intravenous administration.

54. The method of any of claims 2-53, wherein the anti-PD-1 antibody or antigen-binding fragment is administered to the patient by intravenous or subcutaneous administration.

55. The method or pharmaceutical composition of any one of claims 2-54, wherein the pharmaceutical composition comprises an amount of anti-ILT3 antigen binding protein or antigen binding fragment selected from the group consisting of: 7.5 mg; 25 mg; 75 mg; 225 mg; 750 mg; and 2250 mg.

56. The method or pharmaceutical composition of claim 55, wherein the amount of anti-ILT3 antigen binding protein or antigen binding fragment is 7.5 mg.

57. The method or pharmaceutical composition of claim 55, wherein the amount of anti-ILT3 antigen binding protein or antigen binding fragment is 25 mg.

58. The method or pharmaceutical composition of claim 55, wherein the amount of anti-ILT3 antigen binding protein or antigen binding fragment is 75 mg.

59. The method or pharmaceutical composition of claim 55, wherein the amount of anti-ILT3 antigen binding protein or antigen binding fragment is 225 mg.

60. The method or pharmaceutical composition of claim 55, wherein the amount of anti-ILT3 antigen binding protein or antigen binding fragment is 750 mg.

61. The method or pharmaceutical composition of claim 55, wherein the amount of anti-ILT3 antigen binding protein or antigen binding fragment is 2250 mg.

62. The method or pharmaceutical composition of any one of claims 2-61, wherein the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein:
(a) the HC-CDR1 comprises the amino acid sequence set forth in SEQ ID NO: 15; the HC-CDR2 comprises the amino acid sequence set forth in SEQ ID NO: 17; the HC-CDR3 comprises the amino acid sequence set forth in SEQ ID NO: 21; the LC-CDR1 comprises the amino acid sequence set forth in SEQ ID NO: 36; the LC-CDR2 comprises the amino acid sequence set forth in SEQ ID NO: 41; and the LC-CDR3 comprises the amino acid sequence set forth in SEQ ID NO: 42;
(b) the HC-CDR1 has the amino acid sequence set forth in SEQ ID NO: 15; the HC-CDR2 has the amino acid sequence set forth in SEQ ID NO: 18; the HC-CDR3 has the amino acid sequence set forth in SEQ ID NO: 21; the LC-CDR1 has the amino acid sequence set forth in SEQ ID NO: 37; the LC-CDR2 has the amino acid sequence set forth in SEQ ID NO: 41; and, the LC-CDR3 has the amino acid sequence set forth in SEQ ID NO: 42;
(c) the HC-CDR1 has the amino acid sequence set forth in SEQ ID NO: 15; the HC-CDR2 has the amino acid sequence set forth in SEQ ID NO: 19; the HC-CDR3 has the amino acid sequence set forth in SEQ ID NO: 21; the LC-CDR1 has the amino acid sequence set forth in SEQ ID NO: 38; the LC-CDR2 has the amino acid sequence set forth in SEQ ID NO: 41; and, the LC-CDR3 has the amino acid sequence set forth in SEQ ID NO: 42;
(d) the HC-CDR1 has the amino acid sequence set forth in SEQ ID NO: 15; the HC-CDR2 has the amino acid sequence set forth in SEQ ID NO: 18; the HC-CDR3 has the amino acid sequence set forth in SEQ ID NO: 21; the LC-CDR1 has the amino acid sequence set forth in SEQ ID NO: 39; the LC-CDR2 has the amino acid sequence set forth in SEQ ID NO: 41; and, the LC-CDR3 has the amino acid sequence set forth in SEQ ID NO: 42;
(e) the HC-CDR1 has the amino acid sequence set forth in SEQ ID NO: 15; the HC-CDR2 has the amino acid sequence set forth in SEQ ID NO: 17; the HC-CDR3 has the amino acid sequence set forth in SEQ ID NO: 21; the LC-CDR1 has the amino acid sequence set forth in SEQ ID NO: 40; the LC-CDR2 has the amino acid sequence set forth in SEQ ID NO: 41; and, the LC-CDR3 has the amino acid sequence set forth in SEQ ID NO: 42.

63. The method or pharmaceutical composition of claim 62, wherein the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 comprises the amino acid sequence set forth in SEQ ID NO: 15; the HC-CDR2 comprises the amino acid sequence set forth in SEQ ID NO: 17; the HC-CDR3 comprises the amino acid sequence set forth in SEQ ID NO: 21; the LC-CDR1 comprises the amino acid sequence set forth in SEQ ID NO: 36; the LC-CDR2 comprises the amino acid sequence set forth in SEQ ID NO: 41; and the LC-CDR3 comprises the amino acid sequence set forth in SEQ ID NO: 42.

64. The method or pharmaceutical composition of claim 62, wherein the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 has the amino acid sequence set forth in SEQ ID NO: 15; the HC-CDR2 has the amino acid sequence set forth in SEQ ID NO: 18; the HC-CDR3 has the amino acid sequence set forth in SEQ ID NO: 21; the LC-CDR1 has the amino acid sequence set forth in SEQ ID NO: 37; the LC-CDR2 has the amino acid sequence set forth in SEQ ID NO: 41; and, the LC-CDR3 has the amino acid sequence set forth in SEQ ID NO: 42.

65. The method or pharmaceutical composition of claim 62, wherein the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 has the amino acid sequence set forth in SEQ ID NO: 15; the HC-CDR2 has the amino acid sequence set forth in SEQ ID NO: 19; the HC-CDR3 has the amino acid sequence set forth in SEQ ID NO: 21; the LC-CDR1 has the amino acid sequence set forth in SEQ ID NO: 38; the LC-CDR2 has the amino acid sequence set forth in SEQ ID NO: 41; and, the LC-CDR3 has the amino acid sequence set forth in SEQ ID NO: 42.

66. The method or pharmaceutical composition of claim 62, wherein the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 has the amino acid sequence set forth in SEQ ID NO: 15; the HC-CDR2 has the amino acid sequence set forth in SEQ ID NO: 18; the HC-CDR3 has the amino acid sequence set forth in SEQ ID NO: 21; the LC-CDR1 has the amino acid sequence set forth in SEQ ID NO: 39; the LC-CDR2 has the amino acid sequence set forth in SEQ ID NO: 41; and, the LC-CDR3 has the amino acid sequence set forth in SEQ ID NO: 42.

67. The method or pharmaceutical composition of claim 62, wherein the anti-ILT3 antigen binding protein or antigen binding fragment comprises a heavy chain variable domain complementarity determining regions (HC-CDR) 1, 2, and 3, and light chain variable domain complementarity determining regions (LC-CDR) 1, 2, and 3, wherein: the HC-CDR1 has the amino acid sequence set forth in SEQ ID NO: 15; the HC-CDR2 has the amino acid sequence set forth in SEQ ID NO: 17; the HC-CDR3 has the amino acid sequence set forth in SEQ ID NO: 21; the LC-CDR1 has the amino acid sequence set forth in SEQ ID NO: 40; the LC-CDR2 has the amino acid sequence set forth in SEQ ID NO: 41; and, the LC-CDR3 has the amino acid sequence set forth in SEQ ID NO: 42.

68. The method or pharmaceutical composition of any one of claims 2-61, wherein the anti-ILT3 antigen binding protein or antigen binding fragment comprises:
(a) a heavy chain of SEQ ID NO: 140 and a light chain of SEQ ID NO: 149;
(b) a heavy chain of SEQ ID NO: 146 and a light chain of SEQ ID NO: 151;
(c) a heavy chain of SEQ ID NO: 141 and a light chain of SEQ ID NO: 150;
(d) a heavy chain of SEQ ID NO: 141 and a light chain of SEQ ID NO: 163;
(e) a heavy chain of SEQ ID NO: 144 and a light chain of SEQ ID NO: 150.

69. The method or pharmaceutical composition of claim 68, wherein the anti-ILT3 antigen binding protein or antigen binding fragment comprises a heavy chain of SEQ ID NO: 140 and a light chain of SEQ ID NO: 149.

70. The method or pharmaceutical composition of claim 68, wherein the anti-ILT3 antigen binding protein or antigen binding fragment comprises a heavy chain of SEQ ID NO: 146 and a light chain of SEQ ID NO: 151.

71. The method or pharmaceutical composition of claim 68, wherein the anti-ILT3 antigen binding protein or antigen binding fragment comprises a heavy chain of SEQ ID NO: 141 and a light chain of SEQ ID NO: 150.

72. The method or pharmaceutical composition of claim 68, wherein the anti-ILT3 antigen binding protein or antigen binding fragment comprises a heavy chain of SEQ ID NO: 141 and a light chain of SEQ ID NO: 163.

73. The method or pharmaceutical composition of claim 68, wherein the anti-ILT3 antigen binding protein or antigen binding fragment comprises a heavy chain of SEQ ID NO: 144 and a light chain of SEQ ID NO: 150.

74. A pharmaceutical composition comprising from 0.02 mg to 2250 mg of an anti-ILT3 antigen binding protein or antigen binding fragment and a pharmaceutically acceptable excipient for use in the methods of any one of claims 2-73.

75. Use of a pharmaceutical composition comprising from 0.02 mg to 2250 mg of an anti-ILT3 antigen binding protein or antigen binding fragment and a pharmaceutically acceptable excipient in the manufacture of a medicament for use in the methods of any one of claims 2-73.


SEQUENCE LISTING (253 sequences)

SEQ ID NO: 1 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 112 aa, Artificial Sequence
  Description of Artificial Sequence Synthetic polypeptide
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCQQSYDVVTFGAGTKLELK

SEQ ID NO: 3 - protein, 122 aa, Artificial Sequence
  Description of Artificial Sequence Synthetic polypeptide
QVQVQQSGAELAEPGASVKMSCKASGYIFTSYWMHWLKQRPGQGLEWIGYINPSSDYNEYSEKFMDKATLTADKASTTAYMQLISLTSEDSAVYYCARSGWLVHGDYYFDYWGQGTTLTVSS

SEQ ID NO: 4 - protein, 112 aa, Artificial Sequence
  Description of Artificial Sequence Synthetic polypeptide
DIVMSQSPSSLAVSAGEKVTMTCKSSQSLLHTSTRKNYLAWYQQKPGQSPKLLIYWASTRESGVPDRFTGSGSGTDFTLTISSVQAEDLAVYYCKQSYDVVTFGAGTKLELK

SEQ ID NO: 5 - protein, 122 aa, Artificial Sequence
  Description of Artificial Sequence Synthetic polypeptide
QVHLQQSGAELAKPGASVKMSCKASGYTFTSYWIHWIKQRPGQGLEWIGYINPSSGYHEYNQKFIDKATLTADRSSSTAYMHLTSLTSEDSAVYYCARSGWLIHGDYYFDFWGQGTTLTVSS

SEQ ID NO: 6 - protein, 238 aa, Homo sapiens
QAGPLPKPTLWAEPGSVISWGNSVTIWCQGTLEAREYRLDKEESPAPWDRQNPLEPKNKARFSIPSMTEDYAGRYRCYYRSPVGWSQPSDPLELVMTGAYSKPTLSALPSPLVTSGKSVTLLCQSRSPMDTFLLIKERAAHPLLHLRSEHGAQQHQAEFPMSPVTSVHGGTYRCFSSHGFSHYLLSHPSDPLELIVSGSLEDPRPSPTRSVSTAAGPEDQPLMPTGSVPHSGLRRHWE

SEQ ID NO: 7 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 9 - protein, 330 aa, Homo sapiens [sequence omitted]

SEQ ID NO: 10 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 107 aa, Homo sapiens [sequence omitted]

SEQ ID NO: 13 - protein, 122 aa, Artificial Sequence
  Description of Artificial Sequence Synthetic polypeptide
EVQLVESGGDLVKPGGSLKLSCAASGFTFSNYGMSWVRQTPDRRLEWVATISGGGDYTNYPDSMRGRFTISRDNAKNTLYLQMSSLKSEDTAMYYCGRRLWFRSLYYAMDYWGQGTSVTVSS

SEQ ID NO: 14 - protein, 111 aa, Artificial Sequence
  Description of Artificial Sequence Synthetic polypeptide
NIVLTQSPASLAVSLGQRATISCRASEKVDSFGNSFMHWYQQKPGQPPKLLIYLTSNLDSGVPARFSGSGSRTDFALTIDPVEADDAATYYCQQNNEDPYTFGGGTKLEIK

SEQ ID NO: 15 - protein, 5 aa, Artificial Sequence
  Description of Artificial Sequence Synthetic peptide
NYGMS

SEQ ID NO: 16 - protein, 17 aa, Artificial Sequence
  Description of Artificial Sequence Synthetic peptide
  MOD_RES (15)..(15): M, V, or L
TISGGGDYTNYPDSXRG

SEQ ID NO: 17 - protein, 17 aa, Artificial Sequence
  Description of Artificial Sequence Synthetic peptide
TISGGGDYTNYPDSMRG

SEQ ID NO: 18 - protein, 17 aa, Artificial Sequence
  Description of Artificial Sequence Synthetic peptide
TISGGGDYTNYPDSVRG

SEQ ID NO: 19 - protein, 17 aa, Artificial Sequence
  Description of Artificial Sequence Synthetic peptide
TISGGGDYTNYPDSLRG

SEQ ID NO: 20 - protein, 13 aa, Artificial Sequence
  Description of Artificial Sequence Synthetic peptide
  MOD_RES (3)..(3): W, Y, Q, or F
RLXFRSLYYAMDY

SEQ ID NO: 21 - protein, 13 aa, Artificial Sequence
  Description of Artificial Sequence Synthetic peptide
RLWFRSLYYAMDY

SEQ ID NO: 22 - protein, 13 aa, Artificial Sequence
  Description of Artificial Sequence Synthetic peptide
RLYFRSLYYAMDY

SEQ ID NO: 23 - protein, 13 aa, Artificial Sequence
  Description of Artificial Sequence Synthetic peptide
RLQFRSLYYAMDY

SEQ ID NO: 24 - protein, 13 aa, Artificial Sequence
  Description of Artificial Sequence Synthetic peptide
RLFFRSLYYAMDY

SEQ ID NO: 25 - protein, 15 aa, Artificial Sequence
  Description of Artificial Sequence Synthetic peptide
  MOD_RES (11)..(11): N, D, or Q
  MOD_RES (12)..(12): S, N, or A
RASEKVDSFGXXFMH

SEQ ID NO: 26 - protein, 15 aa, Artificial Sequence
  Description of Artificial Sequence Synthetic peptide
  MOD_RES (12)..(12): S, N, or A
RASEKVDSFGNXFMH

SEQ ID NO: 27 - protein, 15 aa, Artificial Sequence
  Description of Artificial Sequence Synthetic peptide
  MOD_RES (12)..(12): S, N, or A
RASEKVDSFGDXFMH

SEQ ID NO: 28 - protein, 15 aa, Artificial Sequence
  Description of Artificial Sequence Synthetic peptide
  MOD_RES (12)..(12): S, N, or A
RASEKVDSFGQXFMH

SEQ ID NO: 29 - protein, 15 aa, Artificial Sequence
  Description of Artificial Sequence Synthetic peptide
  MOD_RES (11)..(11): N, D, or Q
RASEKVDSFGXSFMH

SEQ ID NO: 30 - protein, 15 aa, Artificial Sequence
  Description of Artificial Sequence Synthetic peptide
  MOD_RES (11)..(11): N, D, or Q
RASEKVDSFGXNFMH

SEQ ID NO: 31 - protein, 15 aa, Artificial Sequence
  Description of Artificial Sequence Synthetic peptide
  MOD_RES (11)..(11): N, D, or Q
RASEKVDSFGXAFMH

SEQ ID NO: 32 - protein, 15 aa, Artificial Sequence
  Description of Artificial Sequence Synthetic peptide
RASEKVDSFGNNFMH

SEQ ID NO: 33 - protein, 15 aa, Artificial Sequence
  Description of Artificial Sequence Synthetic peptide
RASEKVDSFGDNFMH

SEQ ID NO: 34 - protein, 15 aa, Artificial Sequence
  Description of Artificial Sequence Synthetic peptide
RASEKVDSFGQNFMH

SEQ ID NO: 35 - protein, 15 aa, Artificial Sequence
  Description of Artificial Sequence Synthetic peptide
RASEKVDSFGNSFMH

SEQ ID NO: 36 - protein, 15 aa, Artificial Sequence
  Description of Artificial Sequence Synthetic peptide
RASEKVDSFGDSFMH

SEQ ID NO: 37 - protein, 15 aa, Artificial Sequence
  Description of Artificial Sequence Synthetic peptide
RASEKVDSFGNAFMH

SEQ ID NO: 38 - protein, 15 aa, Artificial Sequence
  Description of Artificial Sequence Synthetic peptide
RASEKVDSFGDAFMH

SEQ ID NO: 39 - protein, 15 aa, Artificial Sequence
  Description of Artificial Sequence Synthetic peptide
RASEKVDSFGQSFMH

SEQ ID NO: 40 - protein, 15 aa, Artificial Sequence
  Description of Artificial Sequence Synthetic peptide
RASEKVDSFGQAFMH

SEQ ID NO: 41 - protein, 7 aa, Artificial Sequence
  Description of Artificial Sequence Synthetic peptide
LTSNLDS

SEQ ID NO: 42 - protein, 9 aa, Artificial Sequence
  Description of Artificial Sequence Synthetic peptide
QQNNEDPYT

SEQ ID NO: 43 - protein, 121 aa, Artificial Sequence
  Description of Artificial Sequence Synthetic polypeptide
QVQLKESGPGLVQASETLSLTCTVSGFSLTSYSINWVRQSSGKGPEWMGRFWYDEGIAYNLTLESRLSISGDTSKNQVFLKMNSLRTGDTGTYYCTRDRDTVGITGWFAYWGQGTLVTVSS

SEQ ID NO: 44 - protein, 107 aa, Artificial Sequence
  Description of Artificial Sequence Synthetic polypeptide
ETVMTQSPTSLSASIGERVTLNCKASQSVGVNVDWYQQTPGQSPKLLIYGSANRHTGVPDRFTGSGFGSDFTLTISDVEPEDLGVYYCLQYGSVPYTFGAGTKLELK

SEQ ID NO: 45 - protein, 5 aa, Artificial Sequence
  Description of Artificial Sequence Synthetic peptide
SYSIN

SEQ ID NO: 46 - protein, 16 aa, Artificial Sequence
  Description of Artificial Sequence Synthetic peptide
RFWYDEGIAYNLTLES

SEQ ID NO: 47 - protein, 13 aa, Artificial Sequence
  Description of Artificial Sequence Synthetic peptide
DRDTVGITGWFAY

SEQ ID NO: 48 - protein, 11 aa, Artificial Sequence
  Description of Artificial Sequence Synthetic peptide
KASQSVGVNVD

SEQ ID NO: 49 - protein, 7 aa, Artificial Sequence
  Description of Artificial Sequence Synthetic peptide
GSANRHT

SEQ ID NO: 50 - protein, 9 aa, Artificial Sequence
  Description of Artificial Sequence Synthetic peptide
LQYGSVPYT

SEQ ID NO: 51 - protein, 121 aa, Artificial Sequence
  Description of Artificial Sequence Synthetic polypeptide
QVQLKESGPGLVQASETLSLTCTVSGFSLTNYCVNWVRQPSGKGPEWLGRFWFDEGKAYNLTLESRLSISGDTSKNQVFLRMNSLRADDTGTYYCTRDRDTVGITGWFAYWGQGTLVTVSS

SEQ ID NO: 52 - protein, 107 aa, Artificial Sequence
  Description of Artificial Sequence Synthetic polypeptide
ETVMTQSPTSLSASIGERVTLNCKASQSVGINVDWYQQTPGQSPKLLIYGSANRHTGVPDRFTGSGFGSDFTLTISNVEPEDLGVYYCLQYGSVPYTFGPGTKLELK

SEQ ID NO: 53 - protein, 5 aa, Artificial Sequence
  Description of Artificial Sequence Synthetic peptide
NYCVN

SEQ ID NO: 54 - protein, 16 aa, Artificial Sequence
  Description of Artificial Sequence Synthetic peptide
RFWFDEGKAYNLTLES

SEQ ID NO: 55 - protein, 13 aa, Artificial Sequence
  Description of Artificial Sequence Synthetic peptide
DRDTVGITGWFAY

SEQ ID NO: 56 - protein, 11 aa, Artificial Sequence
  Description of Artificial Sequence Synthetic peptide
KASQSVGINVD

SEQ ID NO: 57 - protein, 7 aa, Artificial Sequence
  Description of Artificial Sequence Synthetic peptide
GSANRHT

SEQ ID NO: 58 - protein, 9 aa, Artificial Sequence
  Description of Artificial Sequence Synthetic peptide
LQYGSVPYT

SEQ ID NO: 59 - protein, 118 aa, Artificial Sequence
  Description of Artificial Sequence Synthetic polypeptide
QVQLQQSGAELMKPGASVKISCKATGYTFRTYWIEWVKQRPGHGLEWIGEILPGNGNTHFNENFKDKATFTADTSSNAAYMQLSSLTSEDSAVYYCVRRLGRGPFDFWGQGTTLTVSS

SEQ ID NO: 60 - protein, 107 aa, Artificial Sequence
  Description of Artificial Sequence Synthetic polypeptide
DIQMTQSPSSLSVSLGGKVTITCKASQDINEYIGWYQRKPGKGPRLLIHYTSTLQSGIPSRFSGSGSGRDYSLSISNLEPEDIATYYCLQYANPLPTFGGGTKLEIK

SEQ ID NO: 61 - protein, 5 aa, Artificial Sequence
  Description of Artificial Sequence Synthetic peptide
TYWIE

SEQ ID NO: 62 - protein, 17 aa, Artificial Sequence
  Description of Artificial Sequence Synthetic peptide
EILPGNGNTHFNENFKD

SEQ ID NO: 63 - protein, 10 aa, Artificial Sequence
  Description of Artificial Sequence Synthetic peptide
RRLGRGPFDF

SEQ ID NO: 64 - protein, 11 aa, Artificial Sequence
  Description of Artificial Sequence Synthetic peptide
KASQDINEYIG

SEQ ID NO: 65 - protein, 7 aa, Artificial Sequence
  Description of Artificial Sequence Synthetic peptide
YTSTLQS

SEQ ID NO: 66 - protein, 9 aa, Artificial Sequence
  Description of Artificial Sequence Synthetic peptide
LQYANPLPT

SEQ ID NO: 67 - protein, 122 aa, Artificial Sequence
  Description of Artificial Sequence Synthetic polypeptide
EVQLVESGGGLVQPGRSMKLSCAASGFTFSNFDMAWVRQAPTRGLEWVSSITYDGGSTSYRDSVKGRFTISRDNAKGTLYLQMDSLRSEDTATYYCTTVESIATISTYFDYWGQGVMVTVSS

SEQ ID NO: 68 - protein, 110 aa, Artificial Sequence
  Description of Artificial Sequence Synthetic polypeptide
DIVLTQSPALAVSLGQRATISCRASQSVSMSRYDLIHWYQQKPGQQPKLLIFRASDLASGIPARFSGSGSGTDFTLTINPVQADDIATYYCQQTRKSPPTFGGGTRLELK

SEQ ID NO: 69 - protein, 5 aa, Artificial Sequence
  Description of Artificial Sequence Synthetic peptide
NFDMA

SEQ ID NO: 70 - protein, 17 aa, Artificial Sequence
  Description of Artificial Sequence Synthetic peptide
SITYDGGSTSYRDSVKG

SEQ ID NO: 71 - protein, 13 aa, Artificial Sequence
  Description of Artificial Sequence Synthetic peptide
VESIATISTYFDY

SEQ ID NO: 72 - protein, 15 aa, Artificial Sequence
  Description of Artificial Sequence Synthetic peptide
RASQSVSMSRYDLIH

SEQ ID NO: 73 - protein, 7 aa, Artificial Sequence
  Description of Artificial Sequence Synthetic peptide
RASDLAS

SEQ ID NO: 74 - protein, 9 aa, Artificial Sequence
  Description of Artificial Sequence Synthetic peptide
QQTRKSPPT

SEQ ID NO: 75 - protein, 121 aa, Artificial Sequence
  Description of Artificial Sequence Synthetic polypeptide
QVQLKESGPGLVQASETLSLTCTVSGFSLTSYCVNWVRQPSGKGPEWLGRFWYDEGKVYNLTLESRLSISGDTSKNQVFLKMNRLRTDDTGTYYCTRDRDTMGITGWFAYWGQGTLVTVSS

SEQ ID NO: 76 - protein, 107 aa, Artificial Sequence
  Description of Artificial Sequence Synthetic polypeptide
ETVMTQSPTSLSASIGERVTLNCKASQSVGINVDWYQQTPGQSPKLLIYGSANRHTGVPDRFTGSGFGSGFTLTISNVEPEDLGVYYCLQYGSVPYTFGPGTKLELK

SEQ ID NO: 77 - protein, 5 aa, Artificial Sequence
  Description of Artificial Sequence Synthetic peptide
SYCVN

SEQ ID NO: 78 - protein, 16 aa, Artificial Sequence
  Description of Artificial Sequence Synthetic peptide
RFWYDEGKVYNLTLES

SEQ ID NO: 79 - protein, 13 aa, Artificial Sequence
  Description of Artificial Sequence Synthetic peptide
DRDTMGITGWFAY

SEQ ID NO: 80 - protein, 11 aa, Artificial Sequence
  Description of Artificial Sequence Synthetic peptide
KASQSVGINVD

SEQ ID NO: 81 - protein, 7 aa, Artificial Sequence
  Description of Artificial Sequence Synthetic peptide
GSANRHT

SEQ ID NO: 82 - protein, 9 aa, Artificial Sequence
  Description of Artificial Sequence Synthetic peptide
LQYGSVPYT

SEQ ID NO: 83 - protein, 118 aa, Artificial Sequence
  Description of Artificial Sequence Synthetic polypeptide
QVQMQQSGTELMKPGASMKISCKATGYTFSTYWIQWIKQRPGHGLEWIGEILPGSGTTNYNENFKGKATFSADTSSNTAYIHLSSLTSEDSAVFYCARRLGRGPFDYWGQGTTLTVSS

SEQ ID NO: 84 - protein, 107 aa, Artificial Sequence
  Description of Artificial Sequence Synthetic polypeptide
DIQMTQSPSSLSASLGGKVTITCEASQDINKHIDWYQHQPGRGPSLLIHYASILQPGIPSRFSGSGSGRDYSFSITSLEPEDIATYYCLQYDNLLPTFGGGTKLEIK

SEQ ID NO: 85 - protein, 5 aa, Artificial Sequence
  Description of Artificial Sequence Synthetic peptide
TYWIQ

SEQ ID NO: 86 - protein, 17 aa, Artificial Sequence
  Description of Artificial Sequence Synthetic peptide
EILPGSGTTNYNENFKG

SEQ ID NO: 87 - protein, 9 aa, Artificial Sequence
  Description of Artificial Sequence Synthetic peptide
RLGRGPFDY

SEQ ID NO: 88 - protein, 11 aa, Artificial Sequence
  Description of Artificial Sequence Synthetic peptide
EASQDINKHID

SEQ ID NO: 89 - protein, 7 aa, Artificial Sequence
  Description of Artificial Sequence Synthetic peptide
YASILQP

SEQ ID NO: 90 - protein, 9 aa, Artificial Sequence
  Description of Artificial Sequence Synthetic peptide
LQYDNLLPT

SEQ ID NO: 91 - protein, 121 aa, Artificial Sequence
  Description of Artificial Sequence Synthetic polypeptide
QVQLKESGPGLVQASETLSLTCTVSGFSLTSYSVNWVRQPSGKGLEWMGRFWYDGGTAYNSTLESRLSISGDTSKNQVFLKMNSLQTDDTGTYYCTRDRDTMGITGWFAYWGQGTLVTVSP

SEQ ID NO: 92 - protein, 107 aa, Artificial Sequence
  Description of Artificial Sequence Synthetic polypeptide
ETVMTQSPTSLSASIGERVTLNCKASQSVGVNVDWYQQTPGQSPKLLIYGSANRHTGVPDRFTGSGFGSDFTLTISNVEPEDLGVYYCLQYGSVPYTFGAGTKLELK

SEQ ID NO: 93 - protein, 5 aa, Artificial Sequence
  Description of Artificial Sequence Synthetic peptide
SYSVN

SEQ ID NO: 94 - protein, 16 aa, Artificial Sequence
  Description of Artificial Sequence Synthetic peptide
RFWYDGGTAYNSTLES

SEQ ID NO: 95 - protein, 13 aa, Artificial Sequence
  Description of Artificial Sequence Synthetic peptide
DRDTMGITGWFAY

SEQ ID NO: 96 - protein, 11 aa, Artificial Sequence
  Description of Artificial Sequence Synthetic peptide
KASQSVGVNVD

SEQ ID NO: 97 - protein, 7 aa, Artificial Sequence
  Description of Artificial Sequence Synthetic peptide
GSANRHT

SEQ ID NO: 98 - protein, 9 aa, Artificial Sequence
  Description of Artificial Sequence Synthetic peptide
LQYGSVPYT

SEQ ID NO: 99 - protein, 121 aa, Artificial Sequence
  Description of Artificial Sequence Synthetic polypeptide
QVQLKESGPGLVQASETLSLTCTVSGFSLTSYCVNWVRQPSGKGPEWLGRFWYDEGKVYNLTLESRLSISGDTSKNQVFLKMNRLRTDDTGTYYCTRDRDTLGITGWFAYWGQGTLVTVSS

SEQ ID NO: 100 - protein, 107 aa, Artificial Sequence; identical to SEQ ID NO: 76
  Description of Artificial Sequence Synthetic polypeptide

SEQ ID NO: 101 - protein, 5 aa, Artificial Sequence
  Description of Artificial Sequence Synthetic peptide
SYCVN

SEQ ID NO: 102 - protein, 16 aa, Artificial Sequence
  Description of Artificial Sequence Synthetic peptide
RFWYDEGKVYNLTLES

SEQ ID NO: 103 - protein, 13 aa, Artificial Sequence
  Description of Artificial Sequence Synthetic peptide
DRDTLGITGWFAY

SEQ ID NO: 104 - protein, 11 aa, Artificial Sequence
  Description of Artificial Sequence Synthetic peptide
KASQSVGINVD

SEQ ID NO: 105 - protein, 7 aa, Artificial Sequence
  Description of Artificial Sequence Synthetic peptide
GSANRHT

SEQ ID NO: 106 - protein, 9 aa, Artificial Sequence
  Description of Artificial Sequence Synthetic peptide
LQYGSVPYT

SEQ ID NO: 107 - protein, 19 aa, Artificial Sequence
  Description of Artificial Sequence Synthetic peptide
MEWSWVFLFFLSVTTGVHS

SEQ ID NO: 108 - protein, 20 aa, Artificial Sequence
  Description of Artificial Sequence Synthetic peptide
MSVPTQVLGLLLLWLTDARC

SEQ ID NO: 109 - protein, 452 aa, Artificial Sequence
  Description of Artificial Sequence Synthetic polypeptide
EVQLVESGGDLVKPGGSLKLSCAASGFTFSNYGMSWVRQTPDRRLEWVATISGGGDYTNYPDSMRGRFTISRDNAKNTLYLQMSSLKSEDTAMYYCGRRLWFRSLYYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 110 - protein, 218 aa, Artificial Sequence
  Description of Artificial Sequence Synthetic polypeptide
NIVLTQSPASLAVSLGQRATISCRASEKVDSFGNSFMHWYQQKPGQPPKLLIYLTSNLDSGVPARFSGSGSRTDFALTIDPVEADDAATYYCQQNNEDPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 111 - protein, 449 aa, Artificial Sequence
  Description of Artificial Sequence Synthetic polypeptide
EVQLVESGGDLVKPGGSLKLSCAASGFTFSNYGMSWVRQTPDRRLEWVATISGGGDYTNYPDSMRGRFTISRDNAKNTLYLQMSSLKSEDTAMYYCGRRLWFRSLYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 112 - protein, 449 aa, Artificial Sequence
  Description of Artificial Sequence Synthetic polypeptide
EVQLVESGGDLVKPGGSLKLSCAASGFTFSNYGMSWVRQTPDRRLEWVATISGGGDYTNYPDSVRGRFTISRDNAKNTLYLQMSSLKSEDTAMYYCGRRLWFRSLYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 113 - protein, 449 aa, Artificial Sequence
  Description of Artificial Sequence Synthetic polypeptide
EVQLVESGGDLVKPGGSLKLSCAASGFTFSNYGMSWVRQTPDRRLEWVATISGGGDYTNYPDSLRGRFTISRDNAKNTLYLQMSSLKSEDTAMYYCGRRLWFRSLYYAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 114 - protein, 218 aa, Artificial Sequence
  Description of Artificial Sequence Synthetic polypeptide
NIVLTQSPASLAVSLGQRATISCRASEKVDSFGNSFMHWYQQKPGQPPKLLIYLTSNLDSGVPARFSGSGSRTDFALTIDPVEADDAATYYCQQNNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 122 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSMRGRFTISRDNAKNSLYLQMNSLRAEDTAVYYCGRRLWFRSLYYAMDYWGQGTLVTVSS

SEQ ID NO: 116 - protein, 122 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLWFRSLYYAMDYWGQGTLVTVSS

SEQ ID NO: 117 - protein, 122 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSLRGRFTISRDNAKNSLYLQMNSLRAEDTAVYYCGRRLWFRSLYYAMDYWGQGTLVTVSS

SEQ ID NO: 118 - protein, 122 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLFFRSLYYAMDYWGQGTLVTVSS

SEQ ID NO: 119 - protein, 122 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLYFRSLYYAMDYWGQGTLVTVSS

SEQ ID NO: 120 - protein, 122 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLQFRSLYYAMDYWGQGTLVTVSS

SEQ ID NO: 121 - protein, 122 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSMRGRFTISRDNAKNSLYLQMNSLKAEDTAVYYCGRRLWFRSLYYAMDYWGQGTLVTVSS

SEQ ID NO: 122 - protein, 122 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLKAEDTAVYYCGRRLWFRSLYYAMDYWGQGTLVTVSS

SEQ ID NO: 123 - protein, 122 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSLRGRFTISRDNAKNSLYLQMNSLKAEDTAVYYCGRRLWFRSLYYAMDYWGQGTLVTVSS

SEQ ID NO: 124 - protein, 111 aa, Artificial Sequence
  Description of Artificial Sequence Synthetic polypeptide
DIVLTQSPDSLAVSLGERATINCRASEKVDSFGNSFMHWYQQKPGQPPKLLIYLTSNLDSGVPDRFSGSGSRTDFTLTISSLQAEDVAVYYCQQNNEDPYTFGQGTKLEIK

SEQ ID NO: 125 - protein, 111 aa, Artificial Sequence
  Description of Artificial Sequence Synthetic polypeptide
DIVLTQSPDSLAVSLGERATINCRASEKVDSFGNSFMHWYQQKPGQPPKLLIYLTSNLDSGVPDRFSGSGSGTDFTLTISSLQAEDVAVYYCQQNNEDPYTFGQGTKLEIK

SEQ ID NO: 126 - protein, 111 aa, Artificial Sequence
  Description of Artificial Sequence Synthetic polypeptide
EIVLTQSPATLSLSPGERATLSCRASEKVDSFGNSFMHWYQQKPGQAPRLLIYLTSNLDSGVPARFSGSGSRTDFTLTISSLEPEDFAVYYCQQNNEDPYTFGQGTKLEIK

SEQ ID NO: 127 - protein, 111 aa, Artificial Sequence
  Description of Artificial Sequence Synthetic polypeptide
EIVLTQSPATLSLSPGERATLSCRASEKVDSFGNSFMHWYQQKPGQAPRLLIYLTSNLDSGIPARFSGSGSGTDFTLTISSLEPEDFAVYYCQQNNEDPYTFGQGTKLEIK

SEQ ID NO: 128 - protein, 111 aa, Artificial Sequence
  Description of Artificial Sequence Synthetic polypeptide
DIQLTQSPSSLSASVGDRVTITCRASEKVDSFGNSFMHWYQQKPGKAPKLLIYLTSNLDSGVPSRFSGSGSGTDFTLTISSLQPEDFATYYCQQNNEDPYTFGQGTKLEIK

SEQ ID NO: 129 - protein, 111 aa, Artificial Sequence
  Description of Artificial Sequence Synthetic polypeptide
DIQMTQSPSSLSASVGDRVTITCRASEKVDSFGNSFMHWYQQKPGKAPKLLIYLTSNLDSGVPSRFSGSGSRTDFTLTISSLQPEDFATYYCQQNNEDPYTFGQGTKLEIK

SEQ ID NO: 130 - protein, 111 aa, Artificial Sequence
  Description of Artificial Sequence Synthetic polypeptide
DIQLTQSPSSLSASVGDRVTITCRASEKVDSFGNSFMHWYQQKPGKAPKLLIYLTSNLDSGVPSRFSGSGSRTDFTLTISSLQPEDFATYYCQQNNEDPYTFGQGTKLEIK

SEQ ID NO: 131 - protein, 111 aa, Artificial Sequence
  Description of Artificial Sequence Synthetic polypeptide
DIQLTQSPSSLSASVGDRVTITCRASEKVDSFGNSFMHWYQQKPGKAPKLLIYLTSNLDSGVPARFSGSGSRTDFTLTISSLQPEDFATYYCQQNNEDPYTFGQGTKLEIK

SEQ ID NO: 132 - protein, 111 aa, Artificial Sequence
  Description of Artificial Sequence Synthetic polypeptide
DIVLTQSPDSLAVSLGERATINCRASEKVDSFGNAFMHWYQQKPGQPPKLLIYLTSNLDSGVPDRFSGSGSGTDFTLTISSLQAEDVAVYYCQQNNEDPYTFGQGTKLEIK

SEQ ID NO: 133 - protein, 111 aa, Artificial Sequence
  Description of Artificial Sequence Synthetic polypeptide
DIVLTQSPDSLAVSLGERATINCRASEKVDSFGNNFMHWYQQKPGQPPKLLIYLTSNLDSGVPDRFSGSGSGTDFTLTISSLQAEDVAVYYCQQNNEDPYTFGQGTKLEIK

SEQ ID NO: 134 - protein, 111 aa, Artificial Sequence
  Description of Artificial Sequence Synthetic polypeptide
DIVLTQSPDSLAVSLGERATINCRASEKVDSFGQSFMHWYQQKPGQPPKLLIYLTSNLDSGVPDRFSGSGSGTDFTLTISSLQAEDVAVYYCQQNNEDPYTFGQGTKLEIK

SEQ ID NO: 135 - protein, 111 aa, Artificial Sequence
  Description of Artificial Sequence Synthetic polypeptide
DIVLTQSPDSLAVSLGERATINCRASEKVDSFGDSFMHWYQQKPGQPPKLLIYLTSNLDSGVPDRFSGSGSGTDFTLTISSLQAEDVAVYYCQQNNEDPYTFGQGTKLEIK

SEQ ID NO: 136 - protein, 111 aa, Artificial Sequence
  Description of Artificial Sequence Synthetic polypeptide
DIQLTQSPSSLSASVGDRVTITCRASEKVDSFGNAFMHWYQQKPGKAPKLLIYLTSNLDSGVPSRFSGSGSGTDFTLTISSLQPEDFATYYCQQNNEDPYTFGQGTKLEIK

SEQ ID NO: 137 - protein, 111 aa, Artificial Sequence
  Description of Artificial Sequence Synthetic polypeptide
DIQLTQSPSSLSASVGDRVTITCRASEKVDSFGNNFMHWYQQKPGKAPKLLIYLTSNLDSGVPSRFSGSGSGTDFTLTISSLQPEDFATYYCQQNNEDPYTFGQGTKLEIK

SEQ ID NO: 138 - protein, 111 aa, Artificial Sequence
  Description of Artificial Sequence Synthetic polypeptide
DIQLTQSPSSLSASVGDRVTITCRASEKVDSFGQSFMHWYQQKPGKAPKLLIYLTSNLDSGVPSRFSGSGSGTDFTLTISSLQPEDFATYYCQQNNEDPYTFGQGTKLEIK

SEQ ID NO: 139 - protein, 111 aa, Artificial Sequence
  Description of Artificial Sequence Synthetic polypeptide
DIQLTQSPSSLSASVGDRVTITCRASEKVDSFGDSFMHWYQQKPGKAPKLLIYLTSNLDSGVPSRFSGSGSGTDFTLTISSLQPEDFATYYCQQNNEDPYTFGQGTKLEIK

SEQ ID NO: 140 - protein, 449 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSMRGRFTISRDNAKNSLYLQMNSLRAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 141 - protein, 449 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 142 - protein, 449 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSLRGRFTISRDNAKNSLYLQMNSLRAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 143 - protein, 449 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLFFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 144 - protein, 449 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLYFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 145 - protein, 449 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLQFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 146 - protein, 449 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSMRGRFTISRDNAKNSLYLQMNSLKAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 147 - protein, 449 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLKAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 148 - protein, 449 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSLRGRFTISRDNAKNSLYLQMNSLKAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 149 - protein, 218 aa, Artificial Sequence
  Description of Artificial Sequence Synthetic polypeptide
DIVLTQSPDSLAVSLGERATINCRASEKVDSFGNSFMHWYQQKPGQPPKLLIYLTSNLDSGVPDRFSGSGSRTDFTLTISSLQAEDVAVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 218 aa, Artificial Sequence
  Description of Artificial Sequence Synthetic polypeptide
DIVLTQSPDSLAVSLGERATINCRASEKVDSFGNSFMHWYQQKPGQPPKLLIYLTSNLDSGVPDRFSGSGSGTDFTLTISSLQAEDVAVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 218 aa, Artificial Sequence
  Description of Artificial Sequence Synthetic polypeptide
EIVLTQSPATLSLSPGERATLSCRASEKVDSFGNSFMHWYQQKPGQAPRLLIYLTSNLDSGVPARFSGSGSRTDFTLTISSLEPEDFAVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 218 aa, Artificial Sequence
  Description of Artificial Sequence Synthetic polypeptide
EIVLTQSPATLSLSPGERATLSCRASEKVDSFGNSFMHWYQQKPGQAPRLLIYLTSNLDSGIPARFSGSGSGTDFTLTISSLEPEDFAVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 153 - protein, 218 aa, Artificial Sequence
  Description of Artificial Sequence Synthetic polypeptide
DIQLTQSPSSLSASVGDRVTITCRASEKVDSFGNSFMHWYQQKPGKAPKLLIYLTSNLDSGVPSRFSGSGSGTDFTLTISSLQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 218 aa, Artificial Sequence
  Description of Artificial Sequence Synthetic polypeptide
DIQMTQSPSSLSASVGDRVTITCRASEKVDSFGNSFMHWYQQKPGKAPKLLIYLTSNLDSGVPSRFSGSGSRTDFTLTISSLQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - protein, 218 aa, Artificial Sequence
  Description of Artificial Sequence Synthetic polypeptide
DIQLTQSPSSLSASVGDRVTITCRASEKVDSFGNSFMHWYQQKPGKAPKLLIYLTSNLDSGVPSRFSGSGSRTDFTLTISSLQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 218 aa, Artificial Sequence
  Description of Artificial Sequence Synthetic polypeptide
DIQLTQSPSSLSASVGDRVTITCRASEKVDSFGNSFMHWYQQKPGKAPKLLIYLTSNLDSGVPARFSGSGSRTDFTLTISSLQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 157 - protein, 218 aa, Artificial Sequence
  Description of Artificial Sequence Synthetic polypeptide
DIVLTQSPDSLAVSLGERATINCRASEKVDSFGNAFMHWYQQKPGQPPKLLIYLTSNLDSGVPDRFSGSGSGTDFTLTISSLQAEDVAVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 158 - protein, 218 aa, Artificial Sequence
  Description of Artificial Sequence Synthetic polypeptide
DIVLTQSPDSLAVSLGERATINCRASEKVDSFGNNFMHWYQQKPGQPPKLLIYLTSNLDSGVPDRFSGSGSGTDFTLTISSLQAEDVAVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 159 - protein, 218 aa, Artificial Sequence
  Description of Artificial Sequence Synthetic polypeptide
DIVLTQSPDSLAVSLGERATINCRASEKVDSFGQSFMHWYQQKPGQPPKLLIYLTSNLDSGVPDRFSGSGSGTDFTLTISSLQAEDVAVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 218 aa, Artificial Sequence
  Description of Artificial Sequence Synthetic polypeptide
DIVLTQSPDSLAVSLGERATINCRASEKVDSFGDSFMHWYQQKPGQPPKLLIYLTSNLDSGVPDRFSGSGSGTDFTLTISSLQAEDVAVYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 161 - protein, 218 aa, Artificial Sequence
  Description of Artificial Sequence Synthetic polypeptide
DIQLTQSPSSLSASVGDRVTITCRASEKVDSFGNAFMHWYQQKPGKAPKLLIYLTSNLDSGVPSRFSGSGSGTDFTLTISSLQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 162 - protein, 218 aa, Artificial Sequence
  Description of Artificial Sequence Synthetic polypeptide
DIQLTQSPSSLSASVGDRVTITCRASEKVDSFGNNFMHWYQQKPGKAPKLLIYLTSNLDSGVPSRFSGSGSGTDFTLTISSLQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 163 - protein, 218 aa, Artificial Sequence
  Description of Artificial Sequence Synthetic polypeptide
DIQLTQSPSSLSASVGDRVTITCRASEKVDSFGQSFMHWYQQKPGKAPKLLIYLTSNLDSGVPSRFSGSGSGTDFTLTISSLQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 164 - protein, 218 aa, Artificial Sequence
  Description of Artificial Sequence Synthetic polypeptide
DIQLTQSPSSLSASVGDRVTITCRASEKVDSFGDSFMHWYQQKPGKAPKLLIYLTSNLDSGVPSRFSGSGSGTDFTLTISSLQPEDFATYYCQQNNEDPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 165 - protein, 452 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSMRGRFTISRDNAKNSLYLQMNSLRAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 452 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 452 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSLRGRFTISRDNAKNSLYLQMNSLRAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 452 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLFFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 452 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLYFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 452 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLQFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 171 - protein, 452 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSMRGRFTISRDNAKNSLYLQMNSLKAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 172 - protein, 452 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLKAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 173 - protein, 452 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSLRGRFTISRDNAKNSLYLQMNSLKAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 174 - protein, 448 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSMRGRFTISRDNAKNSLYLQMNSLRAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 175 - protein, 448 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 176 - protein, 448 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSLRGRFTISRDNAKNSLYLQMNSLRAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 177 - protein, 448 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLFFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 178 - protein, 448 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLYFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 179 - protein, 448 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLQFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 180 - protein, 448 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSMRGRFTISRDNAKNSLYLQMNSLKAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 181 - protein, 448 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLKAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 182 - protein, 448 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSLRGRFTISRDNAKNSLYLQMNSLKAEDTAVYYCGRRLWFRSLYYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 183 - protein, 451 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSMRGRFTISRDNAKNSLYLQMNSLRAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 184 - protein, 451 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 185 - protein, 451 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSLRGRFTISRDNAKNSLYLQMNSLRAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 186 - protein, 451 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLFFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 187 - protein, 451 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLYFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 188 - protein, 451 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLQFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 189 - protein, 451 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSMRGRFTISRDNAKNSLYLQMNSLKAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 190 - protein, 451 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLKAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 191 - protein, 451 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSLRGRFTISRDNAKNSLYLQMNSLKAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 192 - protein, 451 aa, Artificial Sequence
  Description of Artificial Sequence Synthetic polypeptide
QVQLKESGPGLVQASETLSLTCTVSGFSLTSYSINWVRQSSGKGPEWMGRFWYDEGIAYNLTLESRLSISGDTSKNQVFLKMNSLRTGDTGTYYCTRDRDTVGITGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 214 aa, Artificial Sequence
  Description of Artificial Sequence Synthetic polypeptide
ETVMTQSPTSLSASIGERVTLNCKASQSVGVNVDWYQQTPGQSPKLLIYGSANRHTGVPDRFTGSGFGSDFTLTISDVEPEDLGVYYCLQYGSVPYTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 194 - protein, 451 aa, Artificial Sequence
  Description of Artificial Sequence Synthetic polypeptide
QVQLKESGPGLVQASETLSLTCTVSGFSLTNYCVNWVRQPSGKGPEWLGRFWFDEGKAYNLTLESRLSISGDTSKNQVFLRMNSLRADDTGTYYCTRDRDTVGITGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 214 aa, Artificial Sequence
  Description of Artificial Sequence Synthetic polypeptide
ETVMTQSPTSLSASIGERVTLNCKASQSVGINVDWYQQTPGQSPKLLIYGSANRHTGVPDRFTGSGFGSDFTLTISNVEPEDLGVYYCLQYGSVPYTFGPGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 196 - protein, 448 aa, Artificial Sequence
  Description of Artificial Sequence Synthetic polypeptide
QVQLQQSGAELMKPGASVKISCKATGYTFRTYWIEWVKQRPGHGLEWIGEILPGNGNTHFNENFKDKATFTADTSSNAAYMQLSSLTSEDSAVYYCVRRLGRGPFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 197 - protein, 214 aa, Artificial Sequence
  Description of Artificial Sequence Synthetic polypeptide
DIQMTQSPSSLSVSLGGKVTITCKASQDINEYIGWYQRKPGKGPRLLIHYTSTLQSGIPSRFSGSGSGRDYSLSISNLEPEDIATYYCLQYANPLPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 198 - protein, 452 aa, Artificial Sequence
  Description of Artificial Sequence Synthetic polypeptide
EVQLVESGGGLVQPGRSMKLSCAASGFTFSNFDMAWVRQAPTRGLEWVSSITYDGGSTSYRDSVKGRFTISRDNAKGTLYLQMDSLRSEDTATYYCTTVESIATISTYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 217 aa, Artificial Sequence
  Description of Artificial Sequence Synthetic polypeptide
DIVLTQSPALAVSLGQRATISCRASQSVSMSRYDLIHWYQQKPGQQPKLLIFRASDLASGIPARFSGSGSGTDFTLTINPVQADDIATYYCQQTRKSPPTFGGGTRLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 451 aa, Artificial Sequence
  Description of Artificial Sequence Synthetic polypeptide
QVQLKESGPGLVQASETLSLTCTVSGFSLTSYCVNWVRQPSGKGPEWLGRFWYDEGKVYNLTLESRLSISGDTSKNQVFLKMNRLRTDDTGTYYCTRDRDTMGITGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 214 aa, Artificial Sequence
  Description of Artificial Sequence Synthetic polypeptide
ETVMTQSPTSLSASIGERVTLNCKASQSVGINVDWYQQTPGQSPKLLIYGSANRHTGVPDRFTGSGFGSGFTLTISNVEPEDLGVYYCLQYGSVPYTFGPGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - protein, 448 aa, Artificial Sequence
  Description of Artificial Sequence Synthetic polypeptide
QVQMQQSGTELMKPGASMKISCKATGYTFSTYWIQWIKQRPGHGLEWIGEILPGSGTTNYNENFKGKATFSADTSSNTAYIHLSSLTSEDSAVFYCARRLGRGPFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - protein, 214 aa, Artificial Sequence
  Description of Artificial Sequence Synthetic polypeptide
DIQMTQSPSSLSASLGGKVTITCEASQDINKHIDWYQHQPGRGPSLLIHYASILQPGIPSRFSGSGSGRDYSFSITSLEPEDIATYYCLQYDNLLPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 204 - protein, 451 aa, Artificial Sequence
  Description of Artificial Sequence Synthetic polypeptide
QVQLKESGPGLVQASETLSLTCTVSGFSLTSYSVNWVRQPSGKGLEWMGRFWYDGGTAYNSTLESRLSISGDTSKNQVFLKMNSLQTDDTGTYYCTRDRDTMGITGWFAYWGQGTLVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - protein, 214 aa, Artificial Sequence
  Description of Artificial Sequence Synthetic polypeptide
ETVMTQSPTSLSASIGERVTLNCKASQSVGVNVDWYQQTPGQSPKLLIYGSANRHTGVPDRFTGSGFGSDFTLTISNVEPEDLGVYYCLQYGSVPYTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 206 - protein, 451 aa, Artificial Sequence
  Description of Artificial Sequence Synthetic polypeptide
QVQLKESGPGLVQASETLSLTCTVSGFSLTSYCVNWVRQPSGKGPEWLGRFWYDEGKVYNLTLESRLSISGDTSKNQVFLKMNRLRTDDTGTYYCTRDRDTLGITGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 214 aa, Artificial Sequence; identical to SEQ ID NO: 201
  Description of Artificial Sequence Synthetic polypeptide

SEQ ID NO: 208 - protein, 452 aa, Artificial Sequence
  Description of Artificial Sequence Synthetic polypeptide
EVQLVESGGGLVQPGGSLRLSCAASGFTFSNYGMSWVRQAPGKGLEWVATISGGGDYTNYPDSVRGRFTISRDNAKNSLYLQMNSLRAEDTAVYYCGRRLWFRSLYYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 451 aa, Artificial Sequence
  Description of Artificial Sequence Synthetic polypeptide
QVQLKESGPGLVQASETLSLTCTVSGFSLTSYSINWVRQSSGKGPEWMGRFWYDEGIAYNLTLESRLSISGDTSKNQVFLKMNSLRTGDTGTYYCTRDRDTVGITGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 451 aa, Artificial Sequence
  Description of Artificial Sequence Synthetic polypeptide
QVQLKESGPGLVQASETLSLTCTVSGFSLTNYCVNWVRQPSGKGPEWLGRFWFDEGKAYNLTLESRLSISGDTSKNQVFLRMNSLRADDTGTYYCTRDRDTVGITGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - protein, 448 aa, Artificial Sequence
  Description of Artificial Sequence Synthetic polypeptide
QVQLQQSGAELMKPGASVKISCKATGYTFRTYWIEWVKQRPGHGLEWIGEILPGNGNTHFNENFKDKATFTADTSSNAAYMQLSSLTSEDSAVYYCVRRLGRGPFDFWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 452 aa, Artificial Sequence
  Description of Artificial Sequence Synthetic polypeptide
EVQLVESGGGLVQPGRSMKLSCAASGFTFSNFDMAWVRQAPTRGLEWVSSITYDGGSTSYRDSVKGRFTISRDNAKGTLYLQMDSLRSEDTATYYCTTVESIATISTYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 451 aa, Artificial Sequence
  Description of Artificial Sequence Synthetic polypeptide
QVQLKESGPGLVQASETLSLTCTVSGFSLTSYCVNWVRQPSGKGPEWLGRFWYDEGKVYNLTLESRLSISGDTSKNQVFLKMNRLRTDDTGTYYCTRDRDTMGITGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 448 aa, Artificial Sequence
  Description of Artificial Sequence Synthetic polypeptide
QVQMQQSGTELMKPGASMKISCKATGYTFSTYWIQWIKQRPGHGLEWIGEILPGSGTTNYNENFKGKATFSADTSSNTAYIHLSSLTSEDSAVFYCARRLGRGPFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 451 aa, Artificial Sequence
  Description of Artificial Sequence Synthetic polypeptide
QVQLKESGPGLVQASETLSLTCTVSGFSLTSYSVNWVRQPSGKGLEWMGRFWYDGGTAYNSTLESRLSISGDTSKNQVFLKMNSLQTDDTGTYYCTRDRDTMGITGWFAYWGQGTLVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 451 aa, Artificial Sequence
  Description of Artificial Sequence Synthetic polypeptide
QVQLKESGPGLVQASETLSLTCTVSGFSLTSYCVNWVRQPSGKGPEWLGRFWYDEGKVYNLTLESRLSISGDTSKNQVFLKMNRLRTDDTGTYYCTRDRDTLGITGWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 451 aa, Artificial Sequence; identical to SEQ ID NO: 210
  Description of Artificial Sequence Synthetic polypeptide

SEQ ID NO: 219 - protein, 4 aa, Artificial Sequence
  Description of Artificial Sequence Synthetic peptide
  MOD_RES (3)..(3): Any amino acid
FGXG

SEQ ID NO: 220 - protein, 4 aa, Artificial Sequence
  Description of Artificial Sequence Synthetic peptide
  MOD_RES (2)..(4): Any amino acid
CXXX

SEQ ID NO: 221 - protein, 5 aa, Artificial Sequence
  Description of Artificial Sequence Synthetic peptide
LEWIG

SEQ ID NO: 222 - protein, 4 aa, Artificial Sequence
  Description of Artificial Sequence Synthetic peptide
  MOD_RES (3)..(3): Any amino acid
WGXG

SEQ ID NO: 223 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 224 - protein, 15 aa, Artificial Sequence
  Description of Artificial Sequence Synthetic peptide
RASKGVSTSGYSYLH

SEQ ID NO: 225 - protein, 7 aa, Artificial Sequence
  Description of Artificial Sequence Synthetic peptide
LASYLES

SEQ ID NO: 226 - protein, 9 aa, Artificial Sequence
  Description of Artificial Sequence Synthetic peptide
QHSRDLPLT

SEQ ID NO: 227 - protein, 5 aa, Artificial Sequence
  Description of Artificial Sequence Synthetic peptide
NYYMY

SEQ ID NO: 228 - protein, 17 aa, Artificial Sequence
  Description of Artificial Sequence Synthetic peptide
GINPSNGGTNFNEKFKN

SEQ ID NO: 229 - protein, 11 aa, Artificial Sequence
  Description of Artificial Sequence Synthetic peptide
RDYRFDMGFDY

SEQ ID NO: 230 - protein, 15 aa, Artificial Sequence
  Description of Artificial Sequence Synthetic peptide
RASKSVSTSGFSYLH

SEQ ID NO: 231 - protein, 7 aa, Artificial Sequence
  Description of Artificial Sequence Synthetic peptide
LASNLES

SEQ ID NO: 232 - protein, 9 aa, Artificial Sequence
  Description of Artificial Sequence Synthetic peptide
QHSWELPLT

SEQ ID NO: 233 - protein, 5 aa, Artificial Sequence
  Description of Artificial Sequence Synthetic peptide
SYYLY

SEQ ID NO: 234 - protein, 17 aa, Artificial Sequence
  Description of Artificial Sequence Synthetic peptide
GVNPSNGGTNFSEKFKS

SEQ ID NO: 235 - protein, 11 aa, Artificial Sequence
  Description of Artificial Sequence Synthetic peptide
RDSNYDGGFDY

SEQ ID NO: 236 - protein, 120 aa, Artificial Sequence [sequence omitted]

SEQ ID NO: 237 - protein, 111 aa, Artificial Sequence [sequence omitted]

SEQ ID NO: 238 - protein, 111 aa, Artificial Sequence
  Description of Artificial Sequence Synthetic polypeptide
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 239 - protein, 111 aa, Artificial Sequence
  Description of Artificial Sequence Synthetic polypeptide
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 240 - protein, 447 aa, Artificial Sequence
  Description of Artificial Sequence Synthetic polypeptide
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 241 - protein, 218 aa, Artificial Sequence
  Description of Artificial Sequence Synthetic polypeptide
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 242 - protein, 218 aa, Artificial Sequence
  Description of Artificial Sequence Synthetic polypeptide
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 243 - protein, 218 aa, Artificial Sequence
  Description of Artificial Sequence Synthetic polypeptide
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 11 aa, Artificial Sequence
  Description of Artificial Sequence Synthetic peptide
RASQSVSSYLA

SEQ ID NO: 245 - protein, 7 aa, Artificial Sequence
  Description of Artificial Sequence Synthetic peptide
DASNRAT

SEQ ID NO: 246 - protein, 9 aa, Artificial Sequence
  Description of Artificial Sequence Synthetic peptide
QQSSNWPRT

SEQ ID NO: 247 - protein, 107 aa, Artificial Sequence [sequence omitted]

SEQ ID NO: 248 - protein, 214 aa, Artificial Sequence
  Description of Artificial Sequence Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 249 - protein, 5 aa, Artificial Sequence
  Description of Artificial Sequence Synthetic peptide
NSGMH

SEQ ID NO: 250 - protein, 17 aa, Artificial Sequence
  Description of Artificial Sequence Synthetic peptide
VIWYDGSKRYYADSVKG

SEQ ID NO: 251 - protein, 4 aa, Artificial Sequence
  Description of Artificial Sequence Synthetic peptide
NDDY

SEQ ID NO: 252 - protein, 113 aa, Artificial Sequence [sequence omitted]

SEQ ID NO: 253 - protein, 440 aa, Artificial Sequence
  Description of Artificial Sequence Synthetic polypeptide
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK